(19) United States
(12) Patent Application Publication
(10) Pub. No.: US 2023/0345815 A1
(43) Pub. Date: Oct. 26, 2023

(54) METAL-ORGANIC COORDINATION COMPOUND AND METHOD FOR PRODUCING THE SAME

(71) Applicant: beeOLED GMBH, Dresden (DE)

(72) Inventors: Carsten ROTHE, Dresden (DE); Volodymyr SENKOVSKYY, Dresden (DE)

(21) Appl. No.: 18/008,142

(22) PCT Filed: Apr. 16, 2021
(86) PCT No.: PCT/EP2021/059962

§ 371 Date: Dec. 2, 2022

(30) Foreign Application Priority Data
Jun. 4, 2020 (DE) 10 2020 114 790.2

(51) Int. Cl. H10K 85/30; C07F 5/00; C09K 11/06; H10K 71/16; H10K 71/12

(52) U.S. Cl. CPC H10K 85/351; C07F 5/003; C09K 11/06; H10K 71/16; H10K 71/12; C09K 2211/182; C09K 2211/1074

(57) ABSTRACT

A metal-organic coordination compound, wherein the coordination compound comprises at least one divalent lanthanide coordinated by a cyclic organic ligand according to formula 1:

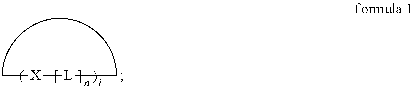

wherein
   i is larger than 3; and
   n is equal to 1, 2, or 3; and
   L for each occurrence is independently selected from
      divalent cyclic organic groups that can be substituted and that are formed by removing two hydrogen atoms from an organic cyclic molecule that can be substituted,
      arylenes, preferably 5- or 6-membered ring aromatic or heteroaromatic group, or
      biradical fragments of

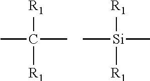

and
X is independently selected for each occurrence from the group of:

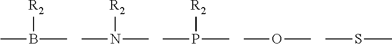

wherein R₁ and R₂ are hydrogen or any covalently bound substituents being identical or different in each occurrence; and
wherein R₁ and/or R₂ are at least in 3 occurrences not hydrogen, and
wherein two groups R₂ can be covalently linked with each other, thereby forming a further cyclic element,
it also being possible that two cyclic organic ligands of formula 1 are covalently linked with each other by one or two divalent linking groups which divalent linking groups are formed of one R₁ of each of the two cyclic organic ligands of formula 1 that are covalently linked with each other.

### TECHNICAL FIELD

Various aspects of this disclosure generally relate to an electroluminescent coordination compound, a mixture, a compound and an organic electronic device and a contrast enhancement medium for magnet resonance tomography having the same as well as methods for forming the same.

### BACKGROUND

Electroluminescent devices that make use of organic light emitting diodes (OLEDs) are state-of-the-art for flat panel display applications used in everyday consumer electronics. For OLEDs usually special organic materials are employed for the purpose of converting electrical excitation into light emission. For most organic emitters, the excitation that is formed upon recombination of an electron and a hole on such an emitter molecule is called an “exciton”. Depending on the spins of the recombining charges, there are two types of excitons formed in a statistical manner: 75% probability of triplet excitons with spin 1, and 25% probability singlet excitons with spin zero are generated. If the emitter molecule is heteroaryl-based without any significant content of heavy metals, then, because of spin-conservation, only the singlet excitons contribute to light emission, known as fluorescence (FL). Thus, fluorescent OLEDs are comparably inefficient as 75% of the invested electrical power is wasted.

In a related art, incorporation of quantum mechanical heavy metal effect into the emitter molecules, by introducing d-metal elements such as Iridium, Osmium, Gold or Platinum, has been used. The presence of heavy metal elements softens the selection rules for the excited states and allows triplet excitons to emit light too; known as phosphorescence (Ph).

In another related art, thermally activated delayed fluorescence (TADF) has been used wherein by thoughtful design of the organic emitter molecule, the energy difference between the non-emissive triplet and the emissive singlet exciton is engineered to be very small. This allows triplets to thermally convert into singlet excitons and thereby contribute to light emission.

However, there are no TADF or Ph blue emitters with sufficient chemical stability known which hinders their implementation into OLED applications. The underlying cause of low chemical stability is due to the formation of charge-separated states upon excitation. For Ph emitters, the exciton resides on two different parts of the emitter, namely the central heavy metal cation and the organic ligand. During excitation, especially with high energetic blue light, the chemical bond between metal center and organic ligand is weakened, giving rise to chemical decomposition. Similarly, for TADF emitters, a low energy difference between singlet and triplet exciton energy is needed, which is achieved by bridging the excitation in-between an electron accepting and an electron donating part. Again, upon excitation with energies corresponding to blue photons, the chemical organic bond between those two parts of the emitter molecules are substantially weakened, giving rise to bond cleavage and in consequence short operational lifetime in OLED device.

Therefore, TADF or Ph blue emitting materials cannot be used in display applications, as otherwise the blue spectral component would fade away after prolonged operation times which is known as burn-in.

For display applications, the emission spectra of the primary red, green, and blue colors should ideally be narrow in order to allow for the highest color purities. Otherwise color filters are used to sharpen the emission spectra, compromising efficiency. In this context, Ph and TADF emitters are not ideal, as they picture rather wide emission spectra. The observed broad emission spectra are an inherent consequence of the design principle based on charge-transfer states being localized between flexible organic bonds, which lead to a wide range of energetic states. Consequently, the implementation of TADF or Ph emitting organic molecules in OLED flat panel display applications, forces the use of color filters, inevitably leading to reduced efficiencies.

Thus, for emitter molecules suitable for OLED, it is desirable to avoid charge separated states, but to localize the excitation on one part of the molecule, preferably on a single atom. Generally, elements with suitable intra-atomic transitions are found within the f-Elements, i.e. the lanthanides. The preferred oxidation state of all lanthanides is 3+. Emitters based on such lanthanides have been extensively used in OLEDs. For example, OLEDs based on intra-atomic transitions in the blue, green, and red spectral region based on Thulium (Tm3+), Terbium (Ter3+) and Europium (Eu3+) respectively have been demonstrated. However, OLED emitters based on three-valent lanthanides have a serious flaw which renders them as being unsuitable for display applications. Here, the excited state relaxation time is around one millisecond, which is about three orders of magnitude too long for display applications. Such slow relaxation times are incompatible with the requirements of fast display content refresh rate and as well lead to a severe efficiency roll-off of the OLEDs at high brightness. Here, a long-excited state lifetime leads to a high density of excitations in the active OLED layer, which leads to bimolecular annihilation loss and low efficiency. Finally, the emission spectra of all three valent lanthanides are not narrow. Instead, a whole range of rather sharp individual narrow lines is distributed over a wide spectral region; which again is not suitable for achieving deep and pure colors.

The above limitations of three valent lanthanides in terms of excited state lifetime and color purity do not apply for specific divalent lanthanides, namely Europium (Eu2+) and Ytterbium (Yb2+), which both possess desired deep blue, narrow emission spectra with sufficiently short excited state lifetime.

Further, Lanthanides with oxidation state+2 are extremely attractive for a large range of applications, mainly due to their unique magnetic and optic properties. For example, Eu(II) is paramagnetic, which can be beneficial for medical diagnostics, memory devices or devices or materials based on magnetic behavior. Divalent Ytterbium and Europium feature attractive emission characteristics due to their Laporte allowed d-f intra-atomic transition. Applied in a right stabilizing environment those ions may emit pure and deep blue light which is applicable for a large range of opto-electronic devices, for example sensors, solar cells, electroluminescent devices, or color conversion materials.

The preferred oxidation state of all lanthanide metals is trivalent. Thus, a major obstacle for the application of divalent lanthanides is their chemical instability, and—in particular—their tendency to oxidize under normal ambient conditions. Therefore, the application of divalent lanthanide requires sufficient stabilization of the cation.

JP3651801B2 discloses the stabilization of lanthanide (II) salts by embedding them into a matrix of inorganic salts. However, due to the high evaporation temperature of inorganic materials, this technique cannot be applied to fabricated state of the art multilayer organic electronic devices, such as OLEDs, due to the unavoidable degradation of underlying organic layer. Further, such inorganic salts are not suitable for injection into the blood system, as required for application as MRT contrast media.

U.S. Pat. No. 6,492,526 discloses a metal organic complex comprising divalent Europium and charged pyrazolyl borate ligands. In this case, the desired stabilization of the divalent oxidation state is achieved by application of the strong electron-donating chelating ligand. Yet, such a strong chelating ligand leads to polarization in the excited state of the central cation. This undesired polarization changes the dominate intra-metallic optical transition of the isolated cation partially into a metal-to-ligand charge transfer state. Thus, excitation energy is transferred to the organic ligand. Consequently, the emission of the compound is substantially red shifted, compared to the desirable deep blue emission from the free Eu2+ cation.

Another related art to combine the desired properties of divalent lanthanides with the benefits of organic processing are metal organic coordination compounds, in which the reactive metal cation is stabilized by cyclic polyether or bicyclic macrocyclic ligands, such as cryptands. In related arts, porphyrins are known as red emitters, specific cryptands have been proposed as ligands for d-metal Ph emitters, crown ether or cryptands have been proposed to stabilize reactive metals for n-type doping, ultraviolet emitting OLED have been achieved by using a Ce(3+) crown ether coordination compound as active emitter. However, in this case, the central metal ion is mainly coordinating to electron rich hard atoms, such as oxygen and nitrogen. Hence, cryptands employing simply hard coordinating atoms such as nitrogen or oxygen cannot sufficiently prevent oxidation of the central reactive ion, such that processing in ambient conditions becomes possible. Thus, no coordination compounds are known that do keep the desired properties of the divalent lanthanide, but at the same time sufficiently prevent oxidation, such that device fabrication or general use in ambient condition becomes possible.

In another related art, a specific strategy to prevent oxidation is known by incorporating soft coordinating ligands, such as sulfur or aromatic aryl or heteroaryl rings into the coordination sphere of the central metal. Thereby oxidation stability of the central metal may be observed. However, in providing such a soft coordination, asymmetrical environment leads to polarization effects in the excited state of the central metal. In other words, the originally pure intra-metallic transition on the central metal ion becomes partly of metal-to-ligand charge transfer (MLCT) type character. Consequently, the originally deep blue and spectrally pure emission shifts substantially to the green and red spectral region and broadens, which renders the application of those metal coordination compounds in opto-electronic devices undesirable. Crown ethers and cryptands with Eu (II) in OLEDs are described in general terms in WO 2004/058912. Nitrogen-containing (macro)circular molecules with Eu (II) in OLEDs, similar to crown ethers, are described in WO 2011/090977. WO 2014/164712 describes nitrogen-containing (macro)circular molecules with Eu (II) for MRI applications. There is no mention of OLED applications. Chem. Commun., 2018, 54, pages 4545-4548, discloses nitrogen-containing (macro)circular molecules with Eu (II). In this respect, (marco)circular molecules are understood to accommodate the central atom, for example Eu (II) in the centre of the circular structure.

### SUMMARY

In various aspects an electroluminescent coordination compound, a mixture, a compound and an organic electronic device and a contrast enhancement medium for magnet resonance tomography having the same as well as methods for forming the same are provided.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various aspects of the invention are described with reference to the following drawings, in which:

FIG. 1A to FIG. 7B illustrate various formulas of coordination compounds according to various aspects;

FIG. 8A and FIG. 8B illustrate schematic cross sections of organic electronic devices according to various aspects;

FIG. 9 illustrates a flow diagram of a method for producing an organic electronic device according to various aspects; and

FIG. 10 illustrates a flow diagram of a method for producing a coordination compound according to various aspects; and

FIG. 11 illustrates formulas of coordination compounds;

FIG. 12 illustrates a formula of a coordination compound according to various embodiments; and

FIG. 13 illustrates a formula for producing a coordination compound according to various embodiments; and

FIG. 14 and FIG. 15 illustrate emission spectra of coordination compounds; and

FIG. 16 and FIG. 17 illustrate formulas of coordination compounds in examples of an organic electronic device.

### DESCRIPTION

The following detailed description refers to the accompanying drawings that show, by way of illustration, specific details and aspects in which the invention may be practiced. Paragraph numbers containing the appendix “A” refer to an amended version intended as an alternative to the same paragraph without appendix “A”.

The word “exemplary” is used herein to mean “serving as an example, instance, or illustration”. Any aspect or design described herein as “exemplary” is not necessarily to be construed as preferred or advantageous over other aspects or designs.

Various embodiments relate to metal organic compounds, including Yb(II) or Eu(II) coordinated with a macrocyclic organic ligand including a plurality of aliphatic amine groups and the applications of those compounds.

In this description, a coordination compound is taken to mean a compound where the central active metal is coordinated without a direct metal carbon bond.

In this description, an electroluminescent coordination compound is any material that is able to emit light upon electrical excitation, followed by recombination of electrons and holes. It shall be irrelevant in this context, whether the recombination of the electrons and holes takes place directly on the electroluminescent compound or first an excitation is formed on a different compound and subsequently transferred to the electroluminescent compound. Further, the electroluminescent coordination compound does not necessarily have to be used in an electronic device but, as example, may be used as a dye or a contrast enhancement medium for magnet resonance tomography.

The divalent lanthanide included in the coordination compound according to various embodiments may be any lanthanide cation that is twofold positively charged, e.g. Yb2+, Eu2+, and Sm2+, in particular Yb2+ and Eu2+.

The macrocyclic organic ligand according to various embodiments may be combined with actinides of divalent oxidation state. For example, Am2+ has a similar electronic configuration to Eu2+ and may therefore have similarly emission properties.

In this description, the arylene is a fragment that is derived from an aromatic or heteroaromatic hydrocarbon that has had a hydrogen atom removed from two adjacent carbon atoms. An aromatic hydrocarbon or arene (or sometimes aryl hydrocarbon) is a hydrocarbon with sigma bonds and delocalized pi electrons between carbon atoms forming a circle.

A In this description, the arylene is a divalent fragment that is derived from an aromatic or heteroaromatic hydrocarbon by removing two hydrogen atoms from the aromatic or heteroaromatic hydrocarbon, preferably from different carbon and/or hetero atoms. One example is a (hetero) aromatic hydrocarbon that has had hydrogen atoms removed from two, preferably adjacent, hydrogen-bearing atoms (in case of aromatic hydrocarbon two carbon atoms, in case of heteroaromatic hydrocarbons two atoms selected from carbon and heteroatoms). An aromatic hydrocarbon or arene (or sometimes aryl hydrocarbon) is a hydrocarbon with sigma bonds and delocalized pi electrons between carbon atoms forming a circle.

The terms “halo,” “halogen,” and “halide” are used interchangeably and refer to fluorine, chlorine, bromine, and iodine.

The term “acyl” refers to a substituted carbonyl radical (C(O)—Rs).

The term “ester” refers to a substituted oxycarbonyl (—O—C(O)—R or —C(O)—O—Rs) radical.

The term “ester” refers to a substituted oxycarbonyl (—O—C(O)—Rs or —C(O)—O—Rs) radical.

The term “ether” refers to an —ORs radical.

The terms “sulfanyl” or “thio-ether” are used interchangeably and refer to a —SRs radical.

The term “sulfinyl” refers to a —S(O)—Rs radical.

The term “sulfonyl” refers to a —SO₂—Rs radical.

The term “phosphino” refers to a —P(Rs)₃ radical, wherein each Rs can be same or different.

The term “silyl” refers to a —Si(Rs)₃ radical, wherein each Rs can be same or different.

In each of the above, Rs can be hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, and combination thereof. Preferred Rs are selected from the group consisting of alkyl, cycloalkyl, aryl, heteroaryl, and combination thereof.

The term “alkyl” refers to and includes both straight and branched chain alkyl radicals. Preferred alkyl groups are those containing from one to fifteen carbon atoms and includes methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. Additionally, the alkyl group is optionally substituted.

The term “alkyl” refers to and includes both straight and branched chain alkyl radicals. Preferred alkyl groups are those containing from one to fifteen, preferably one to ten, more preferably one to five carbon atoms and includes methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. Additionally, the alkyl group is optionally substituted, e.g. by halogen or cycloalkyl.

The term “cycloalkyl” refers to and includes monocyclic, polycyclic, and spiro alkyl radicals. Preferred cycloalkyl groups are those containing 3 to 12 ring carbon atoms and includes cyclopropyl, cyclopentyl, cyclohexyl, bicyclo[3.1.1]heptyl, spiro[4.5]decyl, spiro[5.5]undecyl, adamantyl, and the like. Additionally, the cycloalkyl group is optionally substituted.

The term “cycloalkyl” refers to and includes monocyclic, polycyclic, and spiro alkyl radicals. Preferred cycloalkyl groups are those containing 3 to 12, preferably 3 to 8, more preferably 3 to 6 ring carbon atoms and includes cyclopropyl, cyclopentyl, cyclohexyl, bicyclo[3.1.1]heptyl, spiro[4.5]decyl, spiro[5.5]undecyl, adamantyl, and the like. Additionally, the cycloalkyl group is optionally substituted, e.g. by halogen, alkyl or heteroalkyl.

The terms “heteroalkyl” or “heterocycloalkyl” refer to an alkyl or a cycloalkyl radical, respectively, having at least one carbon atom replaced by a heteroatom. Optionally the at least one heteroatom is selected from O, S, N, P, B, Si and Se, preferably O, S or N. Additionally, the heteroalkyl or heterocycloalkyl group is optionally substituted.

The terms “heteroalkyl” or “heterocycloalkyl” refer to an alkyl or a cycloalkyl radical, respectively, having at least one carbon atom replaced by a heteroatom. Preferably the at least one heteroatom is selected from O, S, N, P, B, Si and Se, more preferably O, S or N. Preferably 1 to 5, more preferably 1 to 3, most preferably 1 or 2 heteroatoms are present in the radical. The radical can be covalently linked with the remainder of the molecule via a carbon or heteroatom (e.g. N). Additionally, the heteroalkyl or heterocycloalkyl group is optionally substituted as indicated for alkyl and cycloalkyl.

The term “alkenyl” refers to and includes both straight and branched chain alkene radicals. Alkenyl groups are essentially alkyl groups that include at least one carbon-carbon double bond in the alkyl chain. Cycloalkenyl groups are essentially cycloalkyl groups that include at least one carbon-carbon double bond in the cycloalkyl ring. The term “heteroalkenyl” as used herein refers to an alkenyl radical having at least one carbon atom replaced by a heteroatom. Optionally the at least one heteroatom is selected from O, S, N, P, B, Si, and Se, preferably O, S, or N. Preferred alkenyl, cycloalkenyl, or heteroalkenyl groups are those containing two to fifteen carbon atoms. Additionally, the alkenyl, cycloalkenyl, or heteroalkenyl group is optionally substituted.

The term “alkenyl” refers to and includes both straight and branched chain alkene radicals. Alkenyl groups are essentially alkyl groups with more than one carbon atom that include at least one carbon-carbon double bond in the alkyl chain. Cycloalkenyl groups are essentially cycloalkyl groups that include at least one carbon-carbon double bond in the cycloalkyl ring. The term “heteroalkenyl” as used herein refers to an alkenyl radical having at least one, preferably 1 to 5, more preferably 1 to 3, most preferably 1 or 2 carbon atom replaced by a heteroatom. Preferably, the at least one heteroatom is selected from O, S, N, P, B, Si, and Se, more preferably O, S, or N. Preferred alkenyl/cycloalkenyl/heteroalkenyl groups are those containing two/three/one to fifteen carbon atoms. Additionally, the alkenyl, cycloalkenyl, or heteroalkenyl group is optionally substituted, as indicated above.

The terms “aralkyl” or “arylalkyl” are used interchangeably and refer to an alkyl group that is substituted with an aryl group. Additionally, the aralkyl group is optionally substituted.

The terms “aralkyl” or “arylalkyl” are used interchangeably and refer to an alkyl group that is substituted with an aryl group. Additionally, the aralkyl group is optionally substituted, as indicated for alkyl and aryl.

The term “heterocyclic group” refers to and includes aromatic and non-aromatic cyclic radicals containing at least one heteroatom. Optionally the at least one heteroatom is selected from O, S, N, P, B, Si, and Se, preferably O, S, or N. Hetero-aromatic cyclic radicals may be used interchangeably with heteroaryl. Preferred hetero-non-aromatic cyclic groups are those containing 3 to 7 ring atoms which includes at least one hetero atom, and includes cyclic amines such as morpholino, piperidino, pyrrolidino, and the like, and cyclic ethers/thio-ethers, such as tetrahydrofuran, tetrahydropyran, tetrahydrothiophene, and the like. Additionally, the heterocyclic group may be optionally substituted.

The term “heterocyclic group” refers to and includes aromatic and non-aromatic cyclic radicals containing at least one, preferably 1 to 5, more preferably 1 to 3, most preferably 1 or 2 heteroatom. Preferably the at least one heteroatom is selected from O, S, N, P, B, Si, and Se, more preferably O, S, or N. Hetero-aromatic cyclic radicals may be used interchangeably with heteroaryl. Preferred hetero-non-aromatic cyclic groups are those containing 3 to 7 ring atoms which includes at least one hetero atom, and includes cyclic amines such as morpholino, piperidino, pyrrolidino, and the like, and cyclic ethers/thio-ethers, such as tetrahydrofuran, tetrahydropyran, tetrahydrothiophene, and the like. Additionally, the heterocyclic group may be optionally substituted, e.g. by halogen, alkyl or aryl. The heterocyclic group can be covalently linked with the remainder of the molecule via carbon and/or heteroatoms, preferably one carbon or nitrogen atom.

The term “aryl” refers to and includes both single-ring aromatic hydrocarbyl groups and polycyclic aromatic ring systems. The polycyclic rings may have two or more rings in which two carbons are common to two adjoining rings (the rings are “fused”) wherein at least one of the rings is an aromatic hydrocarbyl group, e.g., the other rings can be cycloalkyls, cycloalkenyls, aryl, heterocycles, and/or heteroaryls. Preferred aryl groups are those containing six to thirty carbon atoms, preferably six to twenty carbon atoms, more preferably six to twelve carbon atoms. Especially preferred is an aryl group having six carbons, ten carbons or twelve carbons. Suitable aryl groups include phenyl, biphenyl, triphenyl, triphenylene, tetraphenylene, naphthalene, anthracene, phenalene, phenanthrene, fluorene, pyrene, chrysene, perylene, radialene and azulene, preferably phenyl, biphenyl, triphenyl, triphenylene, fluorene, and naphthalene. Additionally, the aryl group is optionally substituted.

The term “aryl” refers to and includes both single-ring aromatic hydrocarbyl groups and polycyclic aromatic ring systems. The polycyclic rings may have two or more rings in which two carbons are common to two adjoining rings (the rings are “fused”) or wherein one carbon is common to two adjoining rings (e.g. biphenyl) wherein at least one of the rings is an aromatic hydrocarbyl group, e.g., the other rings can be cycloalkyls, cycloalkenyls, aryl, heterocycles, and/or heteroaryls. Preferred aryl groups are those containing six to thirty carbon atoms, preferably six to twenty carbon atoms, more preferably six to twelve carbon atoms. Especially preferred is an aryl group having six carbons, ten carbons or twelve carbons. Suitable aryl groups include phenyl, biphenyl, triphenyl, triphenylene, tetraphenylene, naphthalene, anthracene, phenalene, phenanthrene, fluorene, pyrene, chrysene, perylene, radialene and azulene, preferably phenyl, biphenyl, triphenyl, triphenylene, fluorene, and naphthalene. Additionally, the aryl group is optionally substituted, e.g. by halogen, alkyl, heteroalkyl.

The term “heteroaryl” refers to and includes both single-ring aromatic groups and polycyclic aromatic ring systems that include at least one heteroatom. The heteroatoms include, but are not limited to O, S, N, P, B, Si, and Se. In many instances, O, S, or N are the preferred heteroatoms. Hetero-single ring aromatic systems are preferably single rings with 5 or 6 ring atoms, and the ring can have from one to six heteroatoms. The hetero-polycyclic ring systems can have two or more rings in which two atoms are common to two adjoining rings (the rings are “fused”) wherein at least one of the rings is a heteroaryl, e.g., the other rings can be cycloalkyls, cycloalkenyls, aryl, heterocycles, and/or heteroaryls. The hetero-polycyclic aromatic ring systems can have from one to six heteroatoms per ring of the polycyclic aromatic ring system. Preferred heteroaryl groups are those containing three to thirty carbon atoms, preferably three to twenty carbon atoms, more preferably three to twelve carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group is optionally substituted.

The term “heteroaryl” refers to and includes both single-ring aromatic groups and polycyclic aromatic ring systems that include at least one heteroatom. The heteroatoms include, but are not limited to and are preferably selected from 0, 5, N, P, B, Si, and Se. In many instances, O, S, or N are the preferred heteroatoms. Hetero-single ring aromatic systems are preferably single rings with 5 or 6 ring atoms, and the ring can have from one to five/six, preferably 1 to 3, more preferably 1 or 2 heteroatoms. The hetero-polycyclic ring systems can have two or more rings in which two atoms are common to two adjoining rings (the rings are “fused”) or wherein one carbon is common to two adjoining rings (e.g. bipyridine) wherein at least one of the rings is a heteroaryl, e.g., the other rings can be cycloalkyls, cycloalkenyls, aryl, heterocycles, and/or heteroaryls. The hetero-polycyclic aromatic ring systems can have from one to five/six, preferably 1 to 3, more preferably 1 or 2 heteroatoms per ring of the polycyclic aromatic ring system. Preferred heteroaryl groups are those containing three to thirty carbon atoms, preferably three to twenty carbon atoms, more preferably three to twelve carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and is selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof, from which one hydrogen atom has been removed from a hydrogen-bearing carbon or heteroatom to form the covalent link to the remainder of the molecule. Additionally, the heteroaryl group is optionally substituted, e.g. by halogen, alkyl or aryl.

Of the aryl and heteroaryl groups listed above, the groups of triphenylene, naphthalene, anthracene, dibenzothiophene, dibenzofuran, dibenzoselenophene, carbazole, indolocarbazole, imidazole, pyridine, pyrazine, pyrimidine, triazine, and benzimidazole, and the respective aza-analogs of each thereof are of particular interest.

Of the aryl and heteroaryl groups listed above, the groups derived from triphenylene, naphthalene, anthracene, dibenzothiophene, dibenzofuran, dibenzoselenophene, carbazole, indolocarbazole, imidazole, pyridine, pyrazine, pyrimidine, triazine, and benzimidazole, and the respective aza-analogs of each thereof are of particular interest.

The terms alkyl, cycloalkyl, heteroalkyl, heterocycloalkyl, alkenyl, cycloalkenyl, heteroalkenyl, alkynyl, aralkyl, heterocyclic group, aryl, and heteroaryl, as used herein, are independently unsubstituted, or independently substituted, with one or more general substituents.

The alkyl, cycloalkyl, heteroalkyl, heterocycloalkyl, alkenyl, cycloalkenyl, heteroalkenyl, alkynyl, aralkyl, heterocyclic group, aryl, and heteroaryl groups or residues, as used herein, are independently unsubstituted, or independently substituted, with one or more (general) substituents, preferably the substituents mentioned above.

In many instances, the general substituents are selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof.

A Preferably, the (general) substituents are selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof with the number of carbon atoms and is heteroatoms as defined above for the respective term. Furthermore, one or two substituents can be selected from polymer chains which can be covalently linked with the remainder of the molecule by a suitable organic spacer. Therefore, the cyclic organic ligand can be covalently linked with a polymer chain or a polymer backbone.

In some instances, the preferred general substituents are selected from the group consisting of deuterium, fluorine, alkyl, cycloalkyl, heteroalkyl, alkoxy, aryloxy, amino, silyl, alkenyl, cycloalkenyl, heteroalkenyl, aryl, heteroaryl, nitrile, isonitrile, sulfanyl, and combinations thereof.

A In some instances, the preferred general substituents are selected from the group consisting of deuterium, fluorine, alkyl, cycloalkyl, heteroalkyl, alkoxy, aryloxy, amino, silyl, alkenyl, cycloalkenyl, heteroalkenyl, aryl, heteroaryl, nitrile, isonitrile, sulfanyl, and combinations thereof with the number of carbon atoms and heteroatoms as defined above for the respective term.

In some instances, the preferred general substituents are selected from the group consisting of deuterium, fluorine, alkyl, cycloalkyl, alkoxy, aryloxy, amino, silyl, aryl, heteroaryl, sulfanyl, and combinations thereof.

A In some instances, the preferred general substituents are selected from the group consisting of deuterium, fluorine, alkyl, cycloalkyl, alkoxy, aryloxy, amino, silyl, aryl, heteroaryl, sulfanyl, and combinations thereof with the number of carbon atoms and heteroatoms as defined above for the respective term.

In yet other instances, the more preferred general substituents are selected from the group consisting of deuterium, fluorine, alkyl, cycloalkyl, aryl, heteroaryl, and combinations thereof.

A In yet other instances, the more preferred general substituents are selected from the group consisting of deuterium, fluorine, alkyl, cycloalkyl, aryl, heteroaryl, and combinations thereof with the number of carbon atoms and heteroatoms as defined above for the respective term.

The terms “substituted” and “substitution” refer to a substituent other than H that is bonded to the relevant position, e.g., a carbon or nitrogen. For example, when R1 represents mono-substitution, then one R1 must be other than H (i.e., a substitution). Similarly, when R1 represents di-substitution, then two of R1 must be other than H. Similarly, when R1 represents no substitution, R1, for example, can be a hydrogen for available valencies of ring atoms, as in carbon atoms for benzene and the nitrogen atom in pyrrole, or simply represents nothing for ring atoms with fully filled valencies, e.g., the nitrogen atom in pyridine. The maximum number of substitutions possible in a ring structure will depend on the total number of available valencies in the ring atoms.

The terms “substituted” and “substitution” refer to a substituent other than H that is bonded to the relevant position, e.g., a carbon or nitrogen. For example, when R1 represents mono-substitution, then one R1 must be other than H (i.e., a substitution). Similarly, when R1 represents di-substitution, then two of R1 must be other than H. Similarly, when R1 represents no substitution, R1, for example, can be a hydrogen for available valencies of straight or branched chain or ring atoms, as in carbon atoms for benzene and the nitrogen atom in pyrrole, or simply represents nothing for ring atoms with fully filled valencies, e.g., the nitrogen atom in pyridine. The maximum number of substitutions possible in a straight or branched chain or ring structure will depend on the total number of available valencies in the ring atoms or number of hydrogen atoms that can be replaced. All residues and substituents are selected in a way that a chemically stable and accessible chemical group results.

As used herein, “combinations thereof” indicates that one or more members of the applicable list are combined to form a known or chemically stable arrangement that one of ordinary skill in the art can envision from the applicable list. For example, an alkyl and deuterium can be combined to form a partial or fully deuterated alkyl group; a halogen and alkyl can be combined to form a halogenated alkyl substituent; and a halogen, alkyl, and aryl can be combined to form a halogenated arylalkyl. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium. In many instances, a preferred combination of substituent groups will include up to twenty atoms that are not hydrogen or deuterium.

As used herein, “combinations thereof” indicates that one or more members of the applicable list are combined to form a known or chemically stable arrangement that one of ordinary skill in the art can envision from the applicable list. For example, an alkyl and deuterium can be combined to form a partial or fully deuterated alkyl group; a halogen and alkyl can be combined to form a halogenated alkyl substituent; and a halogen, alkyl, and aryl can be combined to form a halogenated arylalkyl. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium counted for all substituents of a given molecule, or for the respective molecule in total. In many instances, a preferred combination of substituent groups will include up to twenty atoms that are not hydrogen or deuterium, counted for all substituents of a given molecule.

The “aza” designation in the fragments described herein, i.e. aza-cryptate, etc. means that one or more of the C—H groups in the respective aromatic ring can be replaced by a nitrogen atom, for example, and without any limitation. One of ordinary skill in the art can readily envision other nitrogen analogs of the aza-derivatives, and all such analogs are intended to be encompassed by the terms as set forth herein.

The “aza” designation in the fragments described herein, i.e. aza-cryptate, etc. means that one or more carbon atom or (other) heteroatom of a parent compound is replaced by a nitrogen atom, without any limitation. For example, in a crown ether —O— is replaced by —NH— to give the respective aza compound. One of ordinary skill in the art can readily envision other nitrogen analogs of the aza-derivatives, and all such analogs are intended to be encompassed by the terms as set forth herein.

As used herein, “deuterium” refers to an isotope of hydrogen. Deuterated compounds can be readily prepared using methods known in the art.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or attached fragment are considered to be equivalent.

In some instances, a pair of adjacent substituents can be optionally joined or fused into a ring. The preferred ring is a five-, six-, or seven-membered carbocyclic or heterocyclic ring, includes both instances where the portion of the ring formed by the pair of substituents is saturated and where the portion of the ring formed by the pair of substituents is unsaturated. As used herein, “adjacent” means that the two substituents involved can be on the same ring next to each other, or on two neighboring rings having the two closest available substitutable positions, such as 2, 2′ positions in a biphenyl, or 1, 8 position in a naphthalene, as long as they can form a stable fused ring system.

A In some instances, a pair of adjacent or non-adjacent substituents or residues can be optionally joined (i.e. covalently linked with each other) or fused into a ring. The preferred ring formed therewith is a five-, six-, or seven-membered carbocyclic or heterocyclic ring, including both instances where the portion of the ring formed by the pair of substituents is saturated and where the portion of the ring formed by the pair of substituents is unsaturated. As used herein, “adjacent” means that the two substituents involved can be on the same ring next to each other, or on two neighboring rings having the two closest available substitutable positions, such as 2, 2′ positions in a biphenyl, or 1, 8 position in a naphthalene, or 2,3-positions in a phenyl, or 1,2-positions in a piperidine, as long as they can form a stable fused ring system.

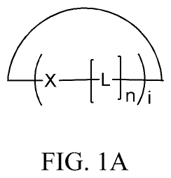

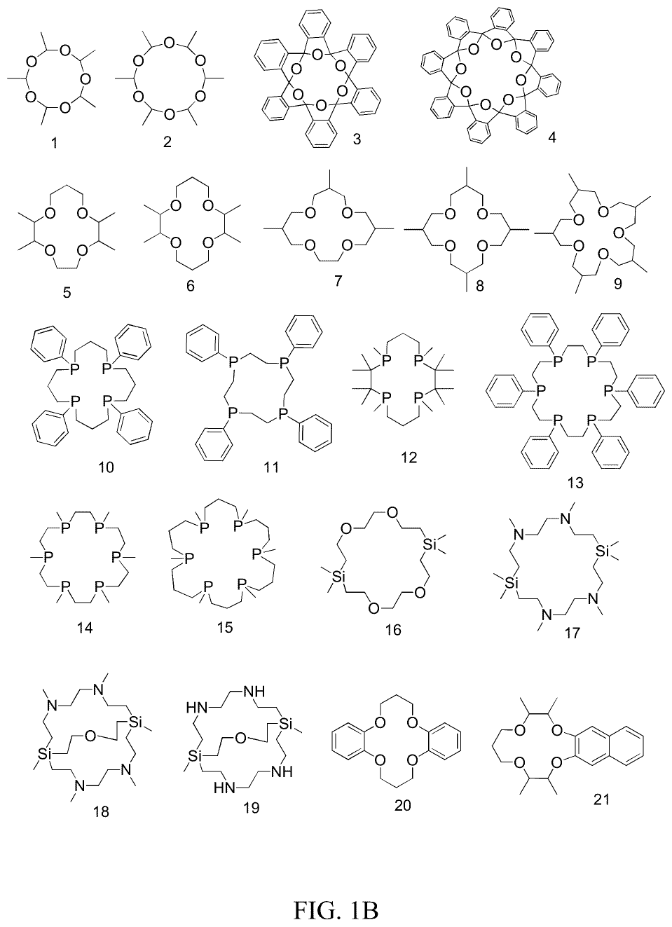

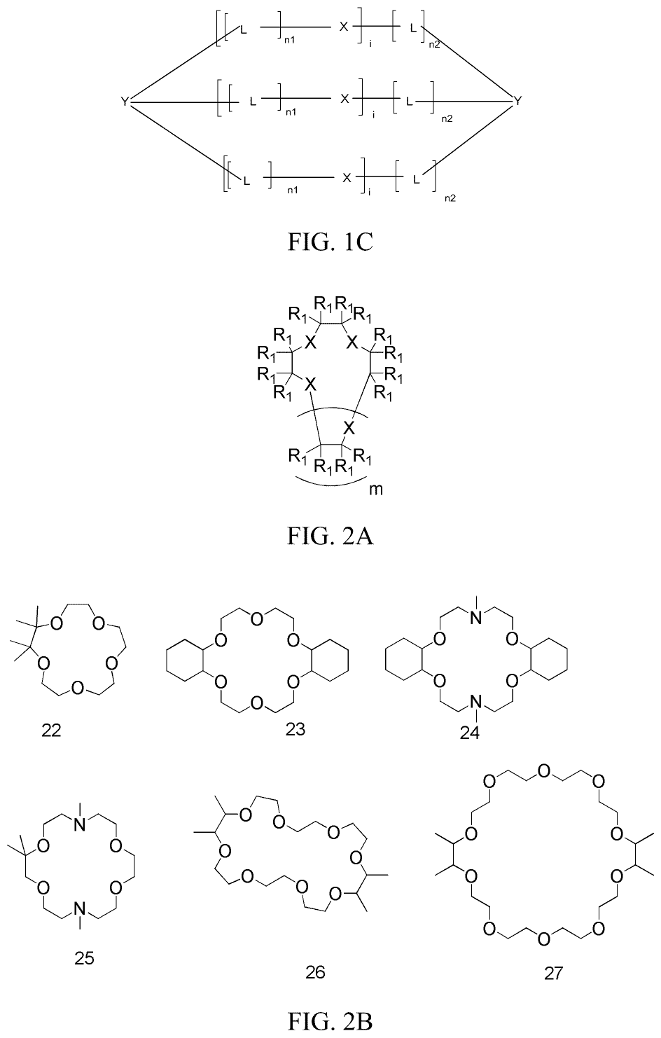

FIG. 1A illustrates a formula of an metal organic coordination compound according to various embodiments. The coordination compound includes at least one divalent lanthanide coordinated by a cyclic organic ligand.

Here, i may be equal to or larger than 3; n may be equal to 1, 2, or 3; and L for each occurrence may be independently selected from arylenes or biradical fragments of

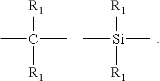

Further, X may be independently selected for each occurrence from the group of:

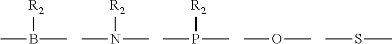

Here, R₁ and R2 may be any covalently bound substituents being identical or different in each occurrence of n and i. R₁ may be independently selected from the group consisting of hydrogen, deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, and combinations thereof. In various embodiments, R1 and/or R2 are at least in 3 occurrences not hydrogen. R1 and R2 may be connected to each other thereby forming a polycyclic ligand or cycloalkyl. In various embodiments, two R2 are connected and, thus, forming an additional bridge and as result the polycyclic compound.

A FIG. 1A illustrates a metal-organic coordination compound, wherein the coordination compound comprises at least one divalent lanthanide coordinated by a cyclic organic ligand according to formula 1:

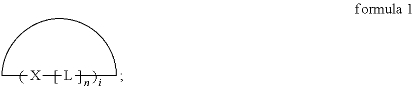

wherein
i is larger than 3; and
n is equal to 1, 2, or 3; and
L for each occurrence is independently selected from
   divalent cyclic organic groups that can be substituted and that are formed by removing two hydrogen atoms from an organic cyclic molecule that can be substituted,
   arylenes, preferably 5- or 6-(membered) ring aromatic or heteroaromatic group, or
   biradical fragments of

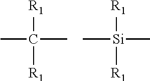

and
X is independently selected for each occurrence from the group of:

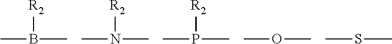

wherein R₁ and R₂ are hydrogen or any covalently bound substituents being identical or different in each occurrence; and
wherein R₁ and/or R₂ are at least in 3 occurrences not hydrogen, and
wherein two groups R₂ can be covalently linked with each other, thereby forming a further cyclic element,
   it also being possible that two cyclic organic ligands of formula 1 are covalently linked with each other by one or two divalent linking groups which divalent linking groups are formed of one R₁ of each of the two cyclic organic ligands of formula 1 that are covalently linked with each other.

This way, oxidation of the atypical divalent into the common trivalent oxidation state is prevented.

Thus, a metal organic coordination compound is provided that is highly stabilized against oxidation such that processing at ambient conditions becomes possible, but at the same time refraining from using soft coordinating ligands such that the beneficial intra-metallic optical transitions of the central metal ion are not shifted into the red spectral region. Illustratively, this is achieved by creating a hard, nitrogen-based coordination sphere for the central divalent ion, and simultaneously preventing oxidation using a plurality of bulky aliphatic substitutions.

Thus, problems according to the related art associated with delocalized excitations are elegantly circumvented using atomic emitters, where the excitation resides substantially on a single atom, giving rise to an atomic excited state. Thus, chemical degradation of the emitting atom itself by excitation is avoided. In various embodiments, the single atom is sufficiently heavy such that both, spin 0 and spin 1 excitations contribute to light emission by means of the heavy metal effect.

In various embodiments, the divalent Lanthanide may be Europium or Ytterbium.

Preferably, the divalent Lanthanide is Europium or Ytterbium.

In various embodiments, the coordination compound may include at least one negatively charged anion, which is not covalently bound to the organic ligand. The negatively charged anion may include more than one atom, preferably more than three atoms, and/or can have a molecular weight of at least 128 g/mol, more preferably at least 180 g/mol. The molecular weight can e.g. be in the range of from 128 to 1000 g/mol, more preferably 180 to 500 g/mol.

Illustratively, the formula illustrated in FIG. 1A describes a chemical structure having a cyclic ring consisting of sub-elements of typically N—C—C or O—C—C or N—C—C—C or O—C—C—C or N—C or O—C. Further, n counts the number of carbons (C), X describes the heteroatom and the index i describes how many of these N—C—C or O—C—C or N—C—C—C or O—C—C—C or N—C or O—C are present. Every C and N may have side groups which may also be linked to themselves. This “linked to itself” may lead to a polycyclic compound which is not illustrated in FIG. 1A. The “linking to itself” may be realized by linking two R's on different C's and, thus, a cryptate is formed. Alternatively, two R's at the same C may be linked (linked to itself) and, thus, realizing a spiro connection. In other words, two of the R1 bond to the same Si or C and thus give a spiro-linkage. Alternatively, either R1 or R2s connect from different atoms, e.g. two R2s connect to form a bridge, e.g. an Aza-cryptate.

A Illustratively, the formula 1 defines a chemical structure having a cyclic ring consisting of sub-elements of formula

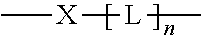

which preferably have a “backbone” formed of a sequence of covalently linked atoms, selected from N—C—C or O—C—C or N—C—C—C or O—C—C—C or N—C or O—C. Further, n counts the number of carbons (C) or divalent cyclic organic groups, X describes the heteroatom and the index i describes how many of these subelements N—C—C or O—C—C or N—C—C—C or O—C—C—C or N—C or O—C are present in the cyclic organic ligand. Every C and N may have side groups which may also be linked to themselves or other C and N atoms of the cyclic organic ligand. This “linked to itself” may lead to a polycyclic compound which is not illustrated in FIG. 1A. The “linking to itself” may be realized by linking two side groups R's on different C's and, thus, a cryptate is formed. Alternatively, two R's at the same C may be linked (linked to itself) and, thus, realizing a spiro connection. In other words, two of the R1 bond to the same Si or C thus give a spiro-linkage. Alternatively, either R1 or R2s connect from different atoms, e.g. two R2s connect to form a bridge, e.g. an Aza-cryptate.

As example, in various embodiments, a macrocycle that may hold the divalent lanthanide may have at least 4 hetero atoms (O or N) and, hence, index i may be greater than 3.

The cyclic organic ligand forming a macrocycle that may hold the divalent lanthanide inside will have at least 4 hetero atoms (e.g. O or N) and, hence, index i will be greater than 3.

In various embodiments, the coordination compound may contain any organic ligand according to FIG. 1A. FIG. 1B illustrates examples 1 to 21 of the organic ligand of FIG. 1A according to various embodiments without limitation.

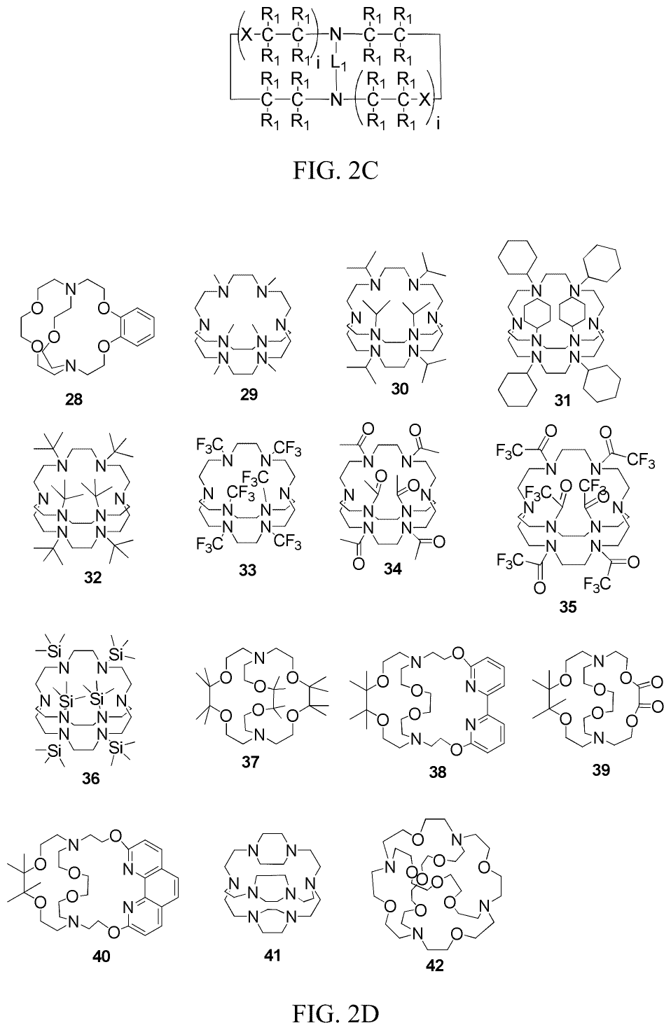

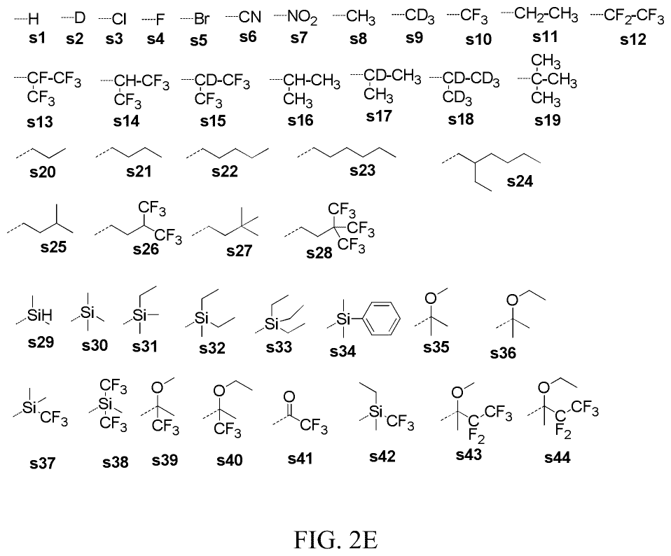

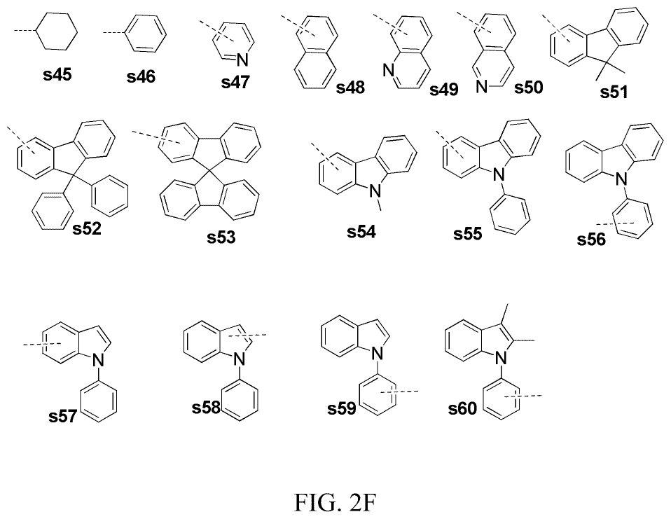

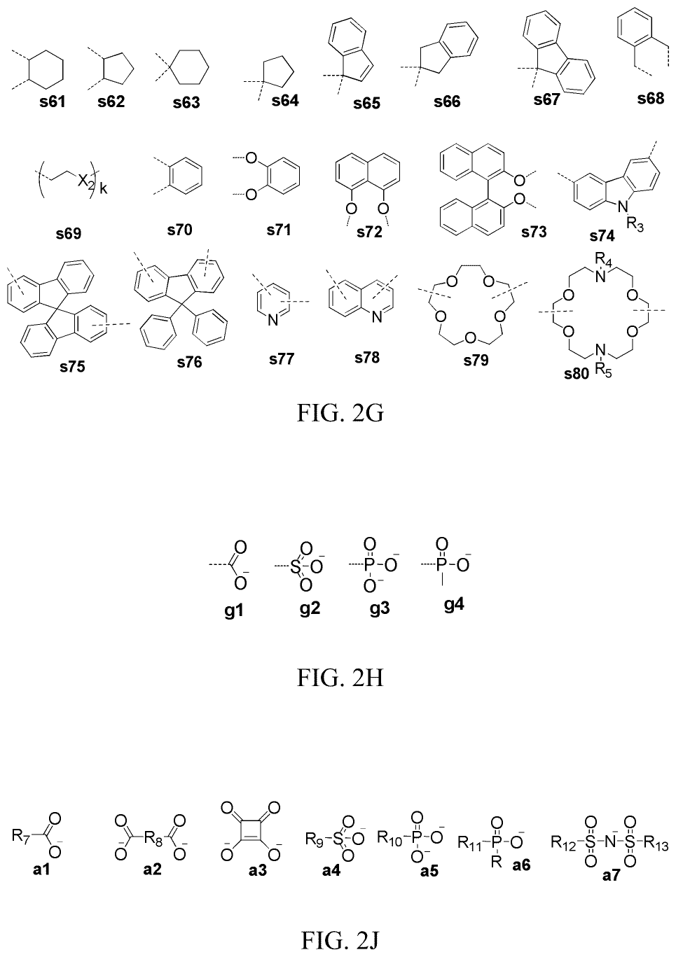

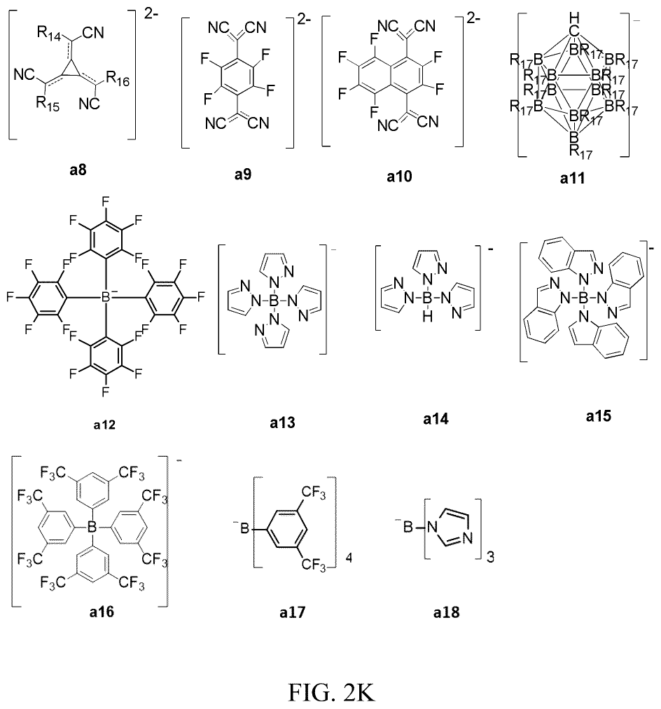

In various embodiments, the coordination compound may include organic ligands according to FIG. 1A with n=2, X=N—R₂ or O and L=C(R₁)₂ described by the generic formula illustrated in FIG. 2A. Here, m may be an integer from 1 to 15, X may be independently in each occurrence O or N—R₂, and R₁ to R₂ may be the same or different in each occurrence. Specific examples according to FIG. 2A, which shall, however, not limit the full scope of the possible set of materials, are compounds 22 to 27 illustrated in FIG. 2B.

A In various embodiments, the coordination compound may include organic ligands according to FIG. 1A with n=2, X being N—R₂ or O and L being C(R₁)₂ described by the generic formula illustrated in FIG. 2A. Here, m preferably is an integer from 1 to 15, X is independently in each occurrence O or N—R₂, and R₁ and R₂ may be the same or different in each occurrence. Specific examples according to FIG. 2A, which shall, however, not limit the full scope of the possible set of materials, are compounds 22 to 27 illustrated in FIG. 2B.

In various embodiments, the coordination compounds include organic ligands according to FIG. 1A which may be configured such that n=2, at least in two instances X=N, and L=C(R₁)₂, whereby at least two of the R₂ that are covalently bound to the nitrogen's themselves form a cyclic ring system, such that the overall ligand according to FIG. 1A describes a polycyclic ligand also known as cryptand. Examples are described by generic formula illustrated in FIG. 2C.

A In various preferred embodiments, the coordination compounds include cyclic organic ligands according to FIG. 1A which are configured such that n=2, at least in two instances X is N—R₂, and L is C(R₁)₂, whereby at least two of the R₂ that are covalently bound to the nitrogens are covalently linked with each other to form a cyclic ring system, so that the overall ligand according to FIG. 1A describes a polycyclic ligand also known as cryptand. Examples are described by generic formula illustrated in FIG. 2C.

Here, L₁ represents the linker, formed from two R₂ substituents, connected to each other at any position. Specific examples according to the formula illustrated in FIG. 2C, which shall not limit the scope, are compounds 28 to 42 illustrated in FIG. 2D.

The coordination compound according to various embodiments may contain a ligand according to FIG. 1A with a plurality of R₁ and R₂ substituents, which are at least in 3 occurrences not equal to hydrogen. Those substituents may be any chemical fragment that can be covalently attached in accordance to the formula illustrated in FIG. 1A. Examples of substituents s1 to s80 are illustrated in FIG. 2E, FIG. 2F and FIG. 2G.

Here, dashed lines may show the preferred covalent attachment to the macrocyclic ligand backbone according to the formula illustrated in FIG. 1A. In various embodiments, the substituents may form any cyclic bridges with each other, and examples, which however shall not limit the scope, are illustrated in FIG. 2F and FIG. 2G.

In FIG. 2F and FIG. 2G, a dashed line may represent the connection of the shown molecular fragment to the macrocyclic backbone of the formula illustrated in FIG. 1A at any position R₁ or R₂ or, in case of two connectors, R₁ and R₂ or R₁ and R₁ or R₂ and R₂. Furthermore, X2 may be selected from 0 or N—R₆ and k may be an integer between 4 and 20 and R₃ to R₆ are selected independently in each occurrence from hydrogen, deuterium, in various embodiments substituted C1-C10 linear or branched alkyl, perfluorinated alkyl, partially fluorinated alkyl, in various embodiments substituted aryl, in various embodiments substituted heteroaryl, perfluorinated aryl, partially fluorinated aryl, in various embodiments substituted cycloalkyl, in various embodiments substituted alkenyl, in various embodiments substituted alkynyl.

In various embodiments, the substituents may include an anionic group. Examples thereof are illustrated in FIG. 2H but are not limited thereto.

In various embodiments, the organic ligand illustrated in FIG. 1A may be configured to be electrically neutral. In various embodiments, the coordination compound may include one or two singly charged anions. As example, in case the organic ligand of the coordination compound of the formula illustrated in FIG. 1A is neutral. In this context, the anion may not be covalently bound to the organic ligand.

When an external field is applied, anions may drift towards an oppositely charged electrodes especially when using a small and non-bulky anion. Such behavior may define a so-called light emitting chemical cell.

A light emitting chemical cell may be an embodiment of an OLED in this description. Drift of charged species within a device including the organic electroluminescent coordination compound according to the formula illustrated in FIG. 1A may lead to very low driving voltages, which may assist to facilitate very good power efficiencies. This may be desirable for some applications, such as general illumination or signage. Yet, for other applications that require fast response times, for example flat panel displays, such ion drift may lead to time dependent OLED characteristics, which may be difficult to control, and may not be desirable as such. Therefore, the choice of the anions may depend strongly on the application. Even without anion drift, a coordination compound according to various embodiments that contain non-covalently bound anions may be organic salts and as such may exhibit an exceptionally large dipole moment. In various embodiments, divalent Europium organic materials may be mild reducing agents; in solid state they may facilitate redox-type charge transfer reactions. Both these properties may facilitate charge injection, either from the electrodes, or from adjacent organic layers in electroluminescent organic devices.

In various embodiments, anions without substantial absorption in the visible spectral region may be used. In case there are two anions, they may be the same or different type.

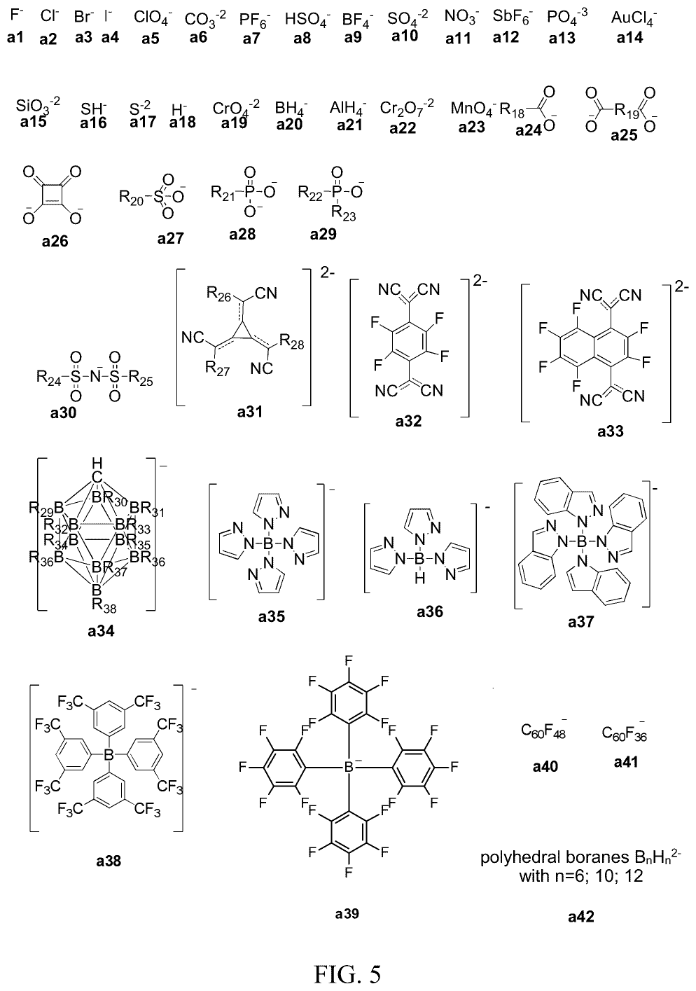

In various embodiments, the coordination compound may contain small inorganic anions such as, but not limited to: F—, Cl—, Br—, I—, ClO₄—, BF₄—, PF₆—, SbF₆—, H—, AlH₄—, BH₄—, NO₃—, SO₄—², HSO₄—, SH—, S²—, CO₃²—, PO₄³—, SiO₃²—, AuCl₄—, CrO₄²—, Cr₂O₇²—, MnO₄——see also a1 to a42 in FIG. 5.

In other embodiments, the coordination compound may contain comparably large anions a1 to a7 as illustrated in FIG. 2J, but are not limited thereto. Here, R₇ may be hydrogen, deuterium, in various embodiments substituted C₁-C₁₀ linear or branched alkyl, perfluorinated alkyl, partially fluorinated alkyl, in various embodiments substituted aryl, perfluorinated aryl, partially fluorinated aryl, in various embodiments substituted cycloalkyl, in various embodiments substituted alkenyl or in various embodiments substituted alkynyl.

A In other embodiments, the coordination compound may contain comparably large anions a1 to a7 as illustrated in FIG. 2J, but are not limited thereto. Here, R₇ preferably is hydrogen, deuterium, (preferably substituted) linear or branched C₁₋₁₀-alkyl, perfluorinated C₁₋₁₀-alkyl, partially fluorinated C₁₋₁₀-alkyl, (preferably substituted) aryl, perfluorinated aryl, partially fluorinated aryl, (preferably substituted) cycloalkyl, substituted alkenyl or i substituted alkynyl.

R₈ may be selected independently from a group including in various embodiments substituted C₁-C₁₀ linear or branched alkanediyls, perfluorinated alkanediyls, partially fluorinated alkanediyls, in various embodiments substituted arylenes, perfluorinated arylenes, partially fluorinated arylenes, in various embodiments substituted cycloalkanediyls in various embodiments substituted alkenediyls or in various embodiments substituted alkyndiyls.

A R₈ is preferably selected independently from a group including (preferably substituted) C₁-C₁₀ linear or branched alkanediyls, perfluorinated alkanediyls, partially fluorinated alkanediyls, (preferably substituted) arylenes, perfluorinated arylenes, partially fluorinated arylenes, (preferably substituted cycloalkanediyls (preferably substituted) alkenediyls or (preferably substituted) alkyndiyls.

R₉ to Ru may be selected independently from a group including hydrogen, deuterium, in various embodiments substituted C₁-C₁₀ linear or branched alkyl, perfluorinated alkyl, partially fluorinated alkyl, in various embodiments substituted aryl, perfluorinated aryl, partially fluorinated aryl, in various embodiments substituted cycloalkyl, in various embodiments substituted alkenyl or in various embodiments substituted alkynyl.

A R₉ to Ru preferably are selected independently from a group including hydrogen, deuterium, (preferably substituted) C₁-C₁₀ linear or branched alkyl, perfluorinated alkyl, partially fluorinated alkyl, (preferably substituted) aryl, perfluorinated aryl, partially fluorinated aryl, (preferably substituted) cycloalkyl, (preferably substituted) alkenyl or (preferably substituted) alkynyl.

R₁₂-R₁₃ may be selected from in various embodiments substituted perfluorinated C₁-C₂₀ alkyl, in various embodiments substituted C₁-C₂₀ alkyl, in various embodiments substituted perfluorinated aryls or in various embodiments substituted aryls.

A R₁₂-R₁₃ preferably are selected from (preferably substituted) perfluorinated C₁-C₂₀ alkyl, (preferably substituted) C₁-C₂₀ alkyl, (preferably substituted) perfluorinated aryls or (preferably substituted) aryls.

In various embodiments, the coordination compound may contain comparably large and bulky organic anions. Such anions may be employed if ion drift may be not desired. Examples of such anions include fluorinated or non-fluorinated fullerene (C₆₀-, C₆₀F₃₆-, C₆₀F₄₈) fluorinated aryl, carboranes, and borates, examples a8 to a8 thereof are illustrated in FIG. 2K but are not limited thereto.

Here, R₁₄ to R₁₆ may be selected independently in each occurrence from a group including of F, CN, in various embodiments substituted perfluorinated aryl or in various embodiments substituted perfluorinated heteroaryl.

A Here, R₁₄ to R₁₆ preferably are selected independently in each occurrence from a group including of F, CN, (preferably substituted) perfluorinated aryl or (preferably substituted) perfluorinated heteroaryl. Preferably, as shown in FIG. 1C, in the metal-organic coordination compound, in which the coordination compound comprises at least one divalent lanthanide coordinated by a cyclic organic ligand, the cyclic organic ligand has the formula 3:

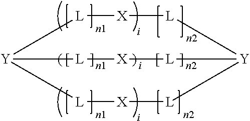

wherein
Y for each occurrence independently is B or B—R₂— or N or P
X is independently selected for each occurrence from the group of:

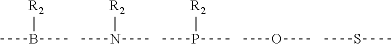

L for each occurrence is independently is a divalent cyclic organic group that can be substituted and that is formed by removing two hydrogen atoms from an organic cyclic molecule that can be substituted, or is a divalent group —CR¹R¹— or —SiR¹R¹— wherein R₁ and R₂ are hydrogen or any covalently bound substituents being identical or different in each occurrence and
n1, n2, i independently are equal to 1, 2, 3, or 4.
Preferably L for each occurrence independently is a divalent cyclic organic group that can be substitued and that is formed by removing two hydrogen atoms from neighboring carbon and/or nitrogen atoms in the ring.
Preferably, n1 and n2 are 1 or 2, more preferably 1, so that the sum of n1+n2 is 2, 3, or 4, more preferably 2 or 3, specifically 2.
Preferably, the compound of formula 3 has one or more of the following features:
Y is B or B—R₂— with R₂ being alkyl, alkoxy, carboxy, aryl, aryloxy or F, or B—H— or N,
all three structural elements

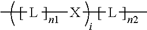

in the compound of formula 3 are identical,
divalent cyclic organic groups L for each occurance independently are divalent cyclic organic groups that can be substituted and that are formed by removing two hydrogen atoms from neighbouring carbon and/or nitrogen atoms in the ring of an organic cyclic molecule that can be substituted, n1 and n2 being 1,
cyclic organic groups are carbocyclic or heterocyclic groups, the heteroatoms being selected from P, N, Si, O, S
if in

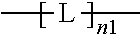

and/or

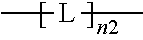

L is —CR¹R¹—, then n1 and/or n2 are 2,

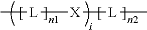

contains two groups X being

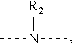

wherein both R₂ together form a group

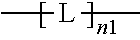

which is identical with group

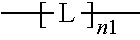

linking the two groups X,
i is 2.
   Therein, any number of the above features can be combined to form a preferred subset of compounds of formula 3. For example, 2, 3 or 4 of the features can be combined to give a more preferred compound of formula 3.

R₁₇ may be selected independently in each occurrence from a group including hydrogen, deuterium, halogen, methyl group, trifluoromethyl-group.

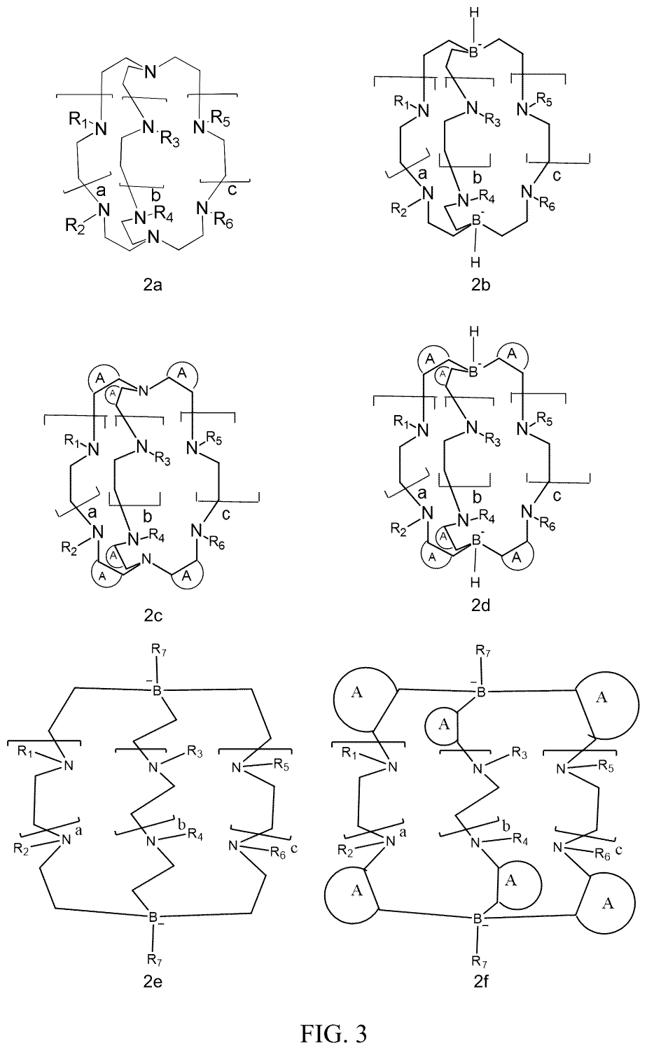

In various embodiments, the coordination compound includes Eu²⁺ or Yb²⁺ and organic ligands that coordinate to Eu²⁺ or Yb²⁺. In other words, Ytterbium or Europium in oxidation state +2 may be coordinated by an organic ligand represented by the formula illustrated in FIG. 3. Thus, FIG. 3 illustrates an embodiment of the cyclic organic ligand illustrated in FIG. 1A-FIG. 2K according to various embodiments.

A Preferably, the coordination compound includes Eu²⁺ or Yb²⁺ and cyclic organic ligands that coordinate to Eu²⁺ or Yb²⁺. In other words, Ytterbium or Europium in oxidation state +2 preferably are coordinated by an organic ligand represented by the formula illustrated in FIG. 3. Thus, FIG. 3 illustrates, preferably in formula 2a, an embodiment of the cyclic organic ligand illustrated in FIG. 1A-FIG. 2K according to the present invention. Preferably, the cyclic organic ligand of formula 1 has a structure according to formula 2a, 2b, 2c, 2d, 2e or 2f, as shown in FIG. 3:

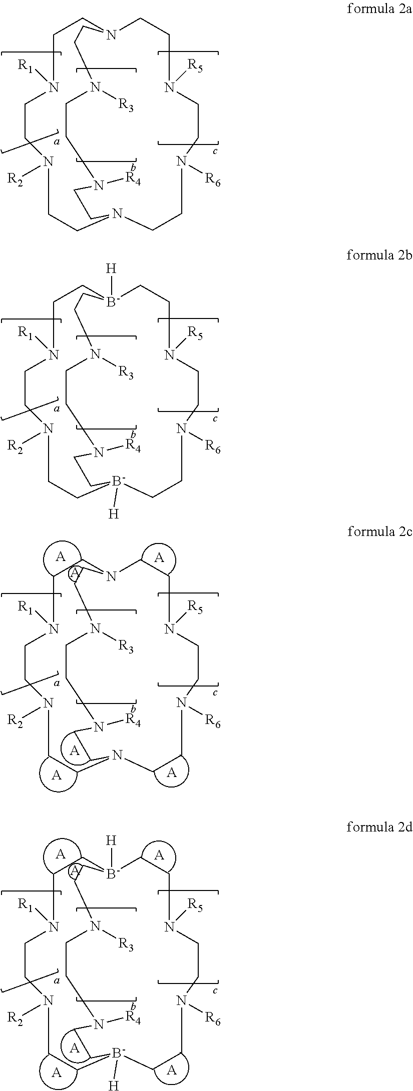

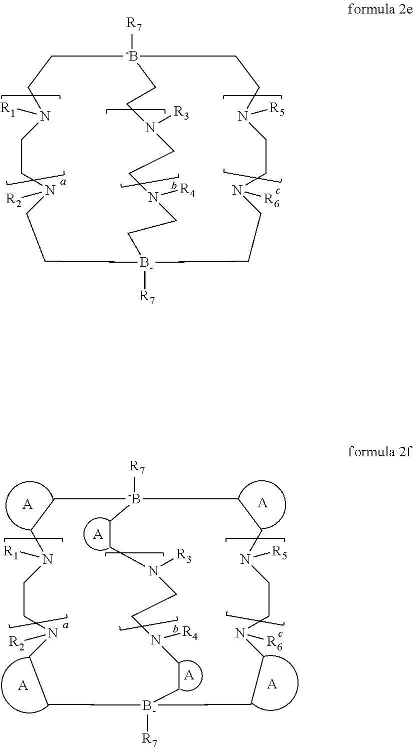

wherein
R₂, R₃, R₄, R₅, R₆ in formula 2a independently in each occurrence represent an organyl group, comprising at least one carbon atom, preferably at least one additional atom different from hydrogen, more preferably at least two carbon atoms, and R₁, R₂, R₃, R₄, R₅, R₆, R₇ in formula 2b, 2c, 2d, 2e, 2f independently in each occurrence represent hydrogen or an organyl group, comprising at least one carbon atom
a, b, c are each independently an integer of 0 or more.

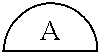

independently in each occurrence represents a divalent cyclic organic group.

The formulae illustrated in FIG. 3, preferably formula 2a, may be derived from the formula illustrated in FIG. 1 when i is equal or larger than 6, two R₂ substituents are connected, forming the bicyclic compound and n is equal to 2 and i of formula in FIG. 1A is equal to a, b and c of FIG. 3, preferably formula 2.

Here, in FIG. 3, preferably in formula 2a, R₁, R₂, R₃, R₄, R₅, R₆ may independently in each occurrence represent an organyl group, including at least one carbon atom and at least one additional atom; whereby the additional atom may be not a hydrogen or R₁, R₂, R₃, R₄, R₅, R₆ may independently in each occurrence represent an organoheteryl group. Further, a, b, c may be each independently an integer of 0 or more. As example, a, b and c may be each equal to 1.

A Here, in FIG. 3, preferably in formula 2a, R₁, R₂, R₃, R₄, R₅, R₆ independently in each occurrence represent an organyl group, including at least one carbon atom and preferably at least one additional atom different from hydrogen, or alternatively R₁, R₂, R₃, R₄, R₅, Re independently in each occurrence represent an organoheteryl group. Further, a, b, c are each independently an integer of 0 or more. As example, a, b and c are each equal to 1.

Thus, the term (electroluminescent) coordination compound may be describing molecules, including metal cations that may be bound to a neutral or a charged ligand represented by the formula illustrated in FIG. 3. If a neutral ligand is used, the coordination compound may be stabilized by an additional anion or pluralities of anions.

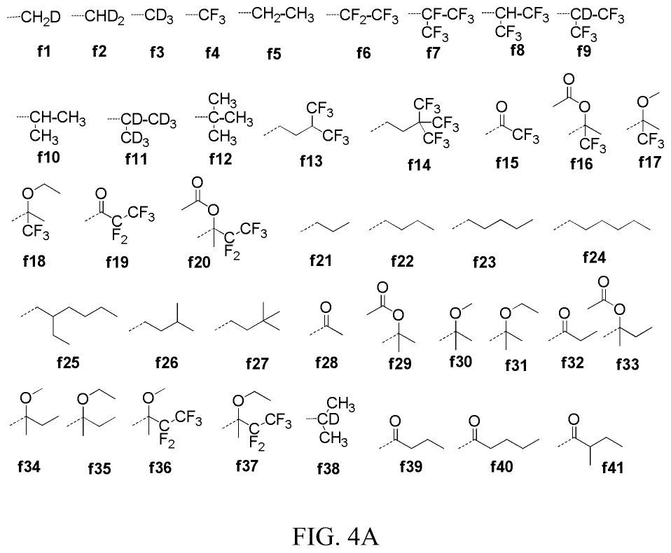

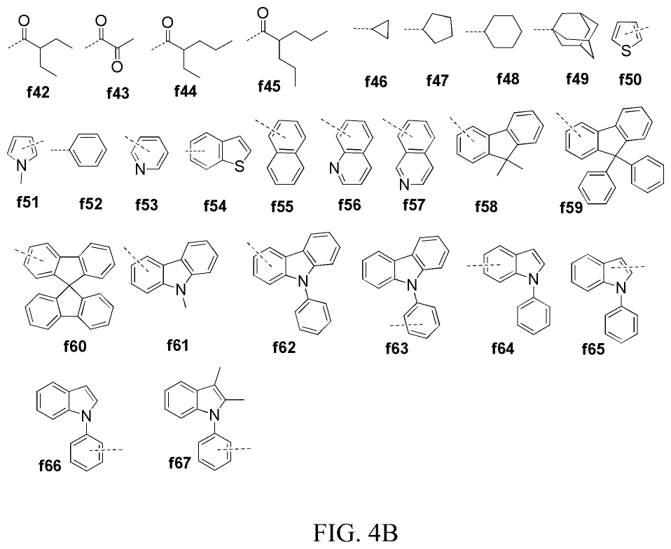

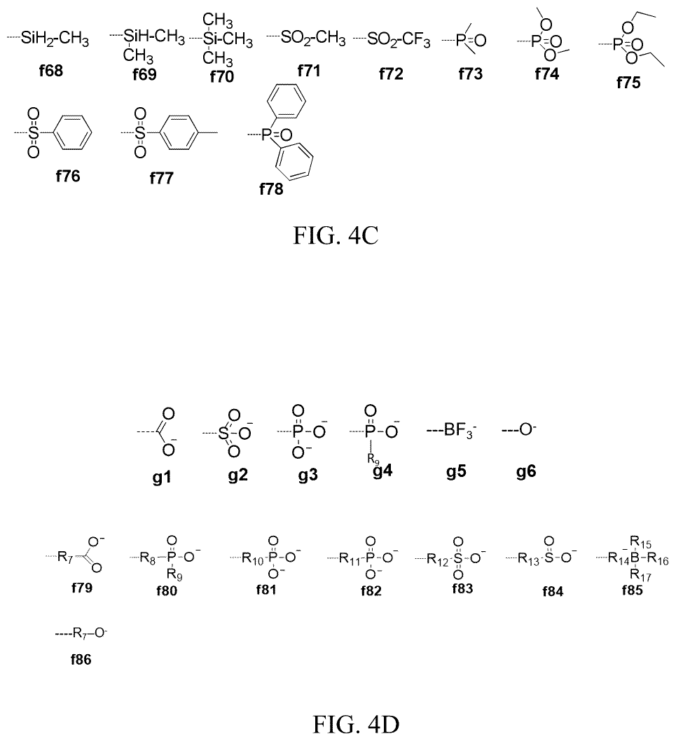

The term organoheteryl group as used herein may define an univalent group including carbon, which may be thus organic, but which may have their free valence at an atom other than carbon. FIG. 4C illustrates the examples of organoheteryl groups f68 to f78, wherein a dashed line may represent preferred connection points. In other words, FIG. 4A to FIG. 4C illustrate chemical formulas of R₁, R₂, R₃, R₄, R₅, R₆ of the cyclic organic ligand illustrated in FIG. 3, preferably formula 2a. R₁, R₂, R₃, R₄, R₅, Re may be in each occurrence independently selected from the group of f1 to f78.

The term organoheteryl group as used herein defines an univalent group including one or more carbon atoms and one or more heteroatoms, which will be thus organic, but which has its free valence at an atom other than carbon. FIG. 4C illustrates the examples of organoheteryl groups f68 to f78, wherein a dashed line may represent preferred connection points. In other words, FIG. 4A to FIG. 4C illustrate chemical formulas of R₁, R₂, R₃, R₄, R₅, Re of the cyclic organic ligand illustrated in FIG. 3, preferably formula 2a. R₁, R₂, R₃, R₄, R₅, Re may be in each occurrence independently selected from the group of f1 to f78.

The term organyl group as used herein may be defined as any organic substituent group, regardless of functional type and constitution, having one free valence at a carbon atom and containing at least one carbon atom, and at least one additional atoms, which may not be hydrogen.

The term organyl group as used herein defines an organic substituent group, regardless of functional type and constitution, having one free valence at a carbon atom and thus containing at least one carbon atom. Preferably, it contains at least one additional atom, which is different from hydrogen.

In various embodiments, the organyl group may include one carbon and one non-hydrogen atoms to 25 carbon atoms, more preferably the organyl group may include 2 to 25 carbon atoms, more preferably the organyl group may include 3 to 25 carbon atoms. FIG. 4A and FIG. 4B illustrate examples of organyl group f1 to f67, wherein a dashed line may represent preferred connection points.

The organyl group preferably includes one to 25 carbon atoms, more preferably the organyl group includes 2 to 25 carbon atoms, more preferably the organyl group includes 3 to 25 carbon atoms. FIG. 4A and FIG. 4B illustrate examples of organyl group f1 to f67, wherein a dashed line may represent preferred connection points.

The value of a, b, c in the formula illustrated in FIG. 3, preferably formula 2a, may be preferably 0 to 5, e.g. in various embodiments 0 to 3, e.g. in various embodiments 0 to 1. In various embodiments, the organyl or organoheteryl group include a charged group, providing a negative charge to the ligand. Examples of charged groups may be presented in FIG. 4D. Here, a dashed line may represent preferred connection points. R₇, R₈, R₁₀, R₁₁, R₁₂, R₁₃, Ru may represent the divalent groups formed by removing of two hydrogen atoms from—in various embodiments substituted alkanes, or in various embodiments substituted arenes or in various embodiments substituted heteroarenes and the free valencies of which are not engaged in a double bond.

The value of a, b, c in the formula illustrated in FIG. 3, preferably formula 2a, is preferably an integer of from 0 to 5, preferably 0 to 3, more preferably 0 to 1. In one embodiment of the invention, the organyl or organoheteryl group includes a charged group, providing a negative charge to the ligand. Examples of charged groups are presented in FIG. 4D. Here, a dashed line represents preferred connection points. R₇, R₈, R₁₀, R₁₁, R₁₂, R₁₃, Ru represent divalent groups formed by removing two hydrogen atoms from (preferably substituted) alkanes, or (preferably substituted) arenes or (preferably substituted) heteroarenes and the free valencies of which are not engaged in a double bond. For example, the coordination compound comprises at least one negatively charged anion, which is covalently bound to the organic ligand, wherein the negatively charged anion is at least one selected from the group of g1 to g6 and f79 to f86, wherein g1 to g6 and f79 to f86 are

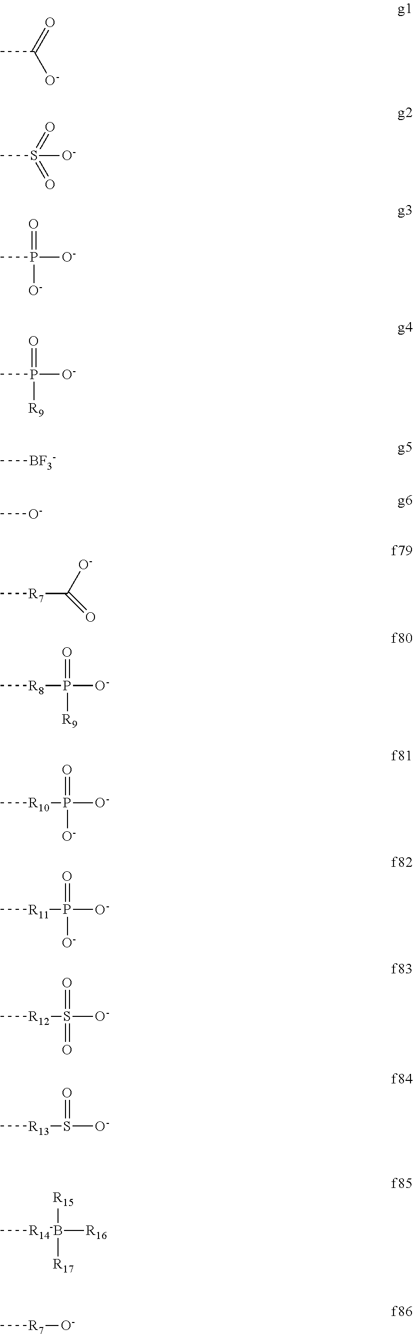

wherein
a dashed line represents a connection point with the organic ligand. R₇, R₈, R₁₀, R₁₁, R₁₂, R₁₃, Ru represent the divalent groups formed by removing of two hydrogen atoms from, preferably substituted, alkanes, or, preferably substituted, arenes or, preferably substituted, heteroarenes and the free valencies of which are not engaged in a double bond,
R₉, R₁₅, R₁₆, R₁₇ represent a monovalent group formed by removing of one hydrogen atom from, preferably substituted, alkanes, or, preferably substituted, arenes, or, preferably substituted, heteroarenes.

The term “in various embodiments substituted” as used herein means that a hydrogen atom, attached to the parent structure could be replaced by a non-hydrogen atom or by a group of atoms. R₉, R₁₅, R₁₆, R₁₇ may represent a monovalent group formed by removing of hydrogen atom from—in various embodiments substituted alkanes, or in various embodiments substituted arenes, or in various embodiments substituted heteroarenes.

The term “substituted” as used herein means that a hydrogen atom, attached to the parent structure is replaced by a non-hydrogen atom or by a group of atoms forming a chemical group. R₉, R₁₅, R₁₆, R₁₇ represent a monovalent group formed by removing a hydrogen atom from (preferably substituted) alkanes, or (preferably substituted) arenes, or (preferably substituted) heteroarenes.

In various embodiments, the coordination compound may include at least one negatively charged anion, which is not covalently bound to the organic ligand. The negatively charged anion may be at least one selected from the group of a1 to a42 illustrated in FIG. 5. Here, R₁₈, R₂₀ to R₃₈ may represent a monovalent group formed by removing of hydrogen atom from—in various embodiments substituted alkanes or in various embodiments substituted arenes, or in various embodiments substituted heteroarenes. Rig may represent the divalent groups formed by removing of two hydrogen atoms from in various embodiments substituted alkanes or in various embodiments substituted arenes or in various embodiments substituted heteroarenes and the free valencies of which may not be engaged in a double bond.

The coordination compound may include at least one negatively charged anion, which is not covalently bound to the cyclic organic ligand. The negatively charged anion may be at least one selected from the group of a1 to a42 illustrated in FIG. 5. Here, R₁₈, R₂₀ to R₃₈ represent a monovalent group formed by removing a hydrogen atom from (preferably substituted) alkanes or (preferably substituted) arenes, or (preferably substituted) heteroarenes. Rig represents a divalent group formed by removing two hydrogen atoms from (preferably substituted) alkanes or (preferably substituted) arenes or (preferably substituted) heteroarenes and the free valencies of which may not be engaged in a double bond.

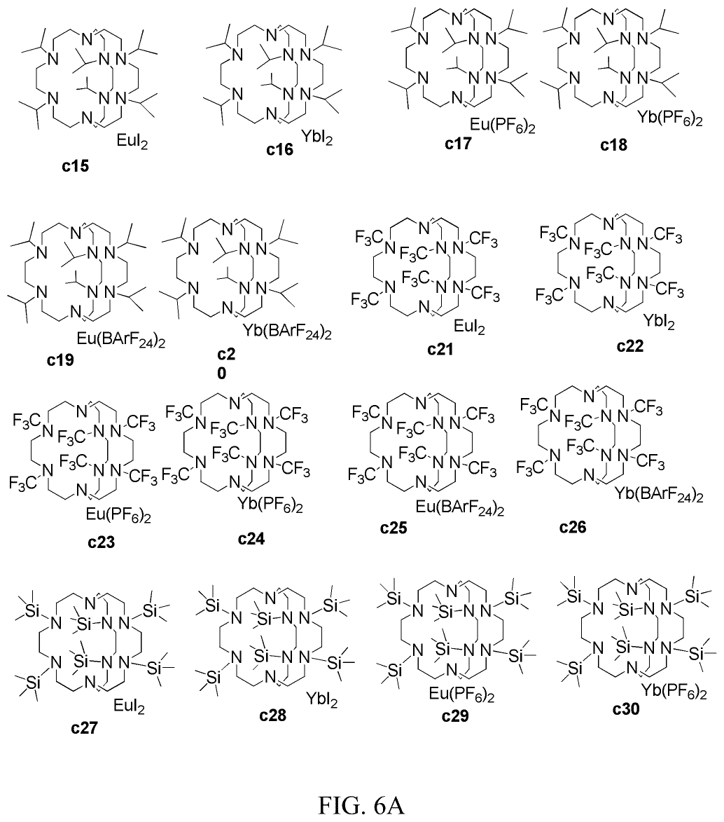

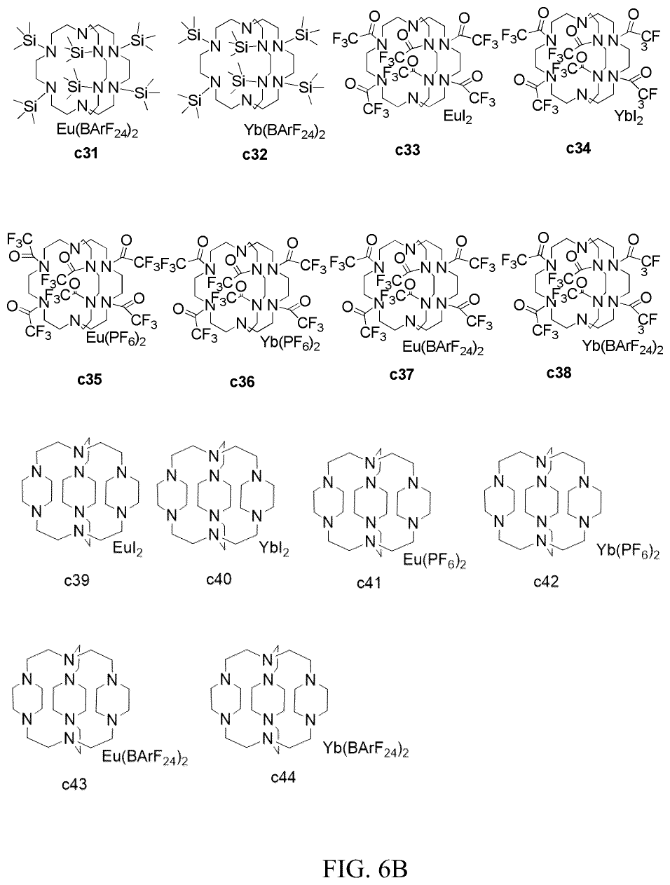

In various embodiments, the coordination compound may be selected from the group of c15 to c44 illustrated in FIG. 6A and FIG. 6B. In FIG. 6A and FIG. 6B, BArF₂₄ represent the anion a38

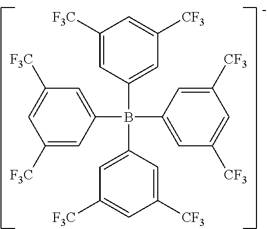

that is also illustrated in FIG. 5.

In various embodiments, the coordination compound according to the invention may be used in a mixture with at least one second electrically neutral or charged organic compound. Preferable for application in electroluminescent devices, the second organic compound may have a triplet energy higher than 2.5 eV. Alternatively or in addition, the coordination compound may have a higher hole affinity compared to the second organic compound.

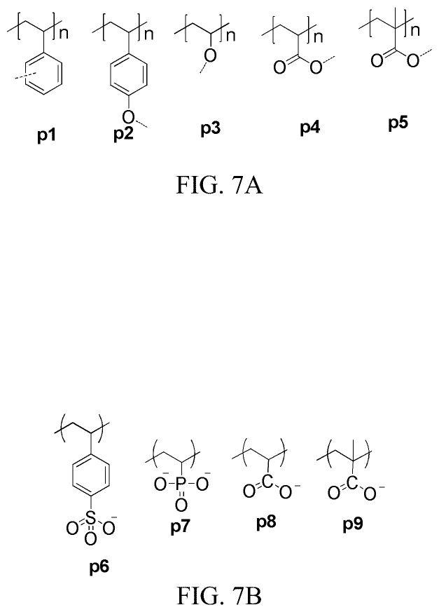

In various embodiments, a compound may include the coordination compound according to any of the described or illustrated embodiments and a polymer with a molecular weight Mn above 1000 g/mol. The coordination compound may be covalently attached to the polymer backbone. The polymer molecule may be an auxiliary organic molecule. In various embodiments, any of the side groups R₁, R₂ of the formula illustrated in FIG. 1A may be linked to another organic molecule, such as a polymer. Some repeat units of polymers that may bind to the coordination compound of formula (1) via R₁ or R₂ are illustrated in FIG. 7A as p1 to p5. Here, a dashed line may represent a preferred connection to R₁ or R₂. It is, however, understood that those are specific examples for illustration purpose only.

In various embodiments, the coordination compound including the organic ligand, according to the formula illustrated in FIG. 1A, may contain one or more non-covalently bound anions covalently bound to a binder molecule of high molecular weight, e.g. to avoid undesired ion drift. The molecular weight of the binder molecule may be larger than 1000 g/mol. As example the binder molecule may be a polymer with more than 2 repeat units. Examples for the binder molecule being a polymer may be given by compounds p6-p9 illustrated in FIG. 7B.

In various embodiments, a contrast enhancement medium for magnet resonance tomography (MRT) may include the coordination compound according to any one of FIG. 1A to FIG. 6B e.g. as a pure compound, in a mixture or a compound as described before. For MRT preference is given for Europium over Ytterbium. The divalent Europium and Ytterbium coordination compound according to various embodiments shows an unmatched stability in solution, e.g. including aqueous solution and even to slightly basic or acidic environment and even at elevated temperatures. Therefore, the coordination compound can be applied in biological specimens, such as inside the blood circulation system. Because of its half-filled f-orbital, divalent Europium has one of the highest local ionic magnetic moments (J=7/2). This moment may interact with surrounding water molecules leading to paramagnetic exchange, making it useful as contrast enhancing agent in magnetic resonance imaging (MRT). In such application, preference may be given to coordination compounds according to various embodiments that include non-covalently bound anions.

In various embodiments, the coordination compound according to various embodiments may be dispersed into a matrix of suitable optical properties, such as for example a high transparency in certain desired parts of the visible spectrum. If this matrix is brought in optical contact to a light source emitting at sufficiently short wavelength, this light may be absorbed by the coordination compound and reemitted at substantially longer wavelength. A suitable light source may for example be a light emitting diode emitting light at wavelength substantially shorter than 430 nm, which may be reemitted by the coordination compound at wavelength longer than 430 nm. The matrix may have any physical dimensions. It may for example be a thin layer of 10 nm to 10,000 nm. The matrix may as well be of granular form or in form of small particles of average diameter between 10 nm and 100,000 nm. For use in optical devices, in the latter cases, the matrix may be applied inside another host material for support.

The combination of unique paramagnetic and optical properties with the ease of processing makes the coordination compound according to various embodiments highly attractive for application in organic electronic devices. In this description an organic electronic device may be any device or structure including substantially organic layers or subunits, where an electro-magnetic stimulus may be converted into an electrical field or current or vice versa, or where an input electrical current or electrical field may be used to modulate an output electrical current or electrical field.

In one embodiment, the organic electronic device including the coordination compound according to various embodiments may be used as semiconducting organic material in an organic field-effect transistor or an organic thin-film transistor.

In another embodiment the organic electronic device converts external static or dynamic electro-magnetic fields into proportional, measurable electrical signals and thus becomes a magnet field sensor.

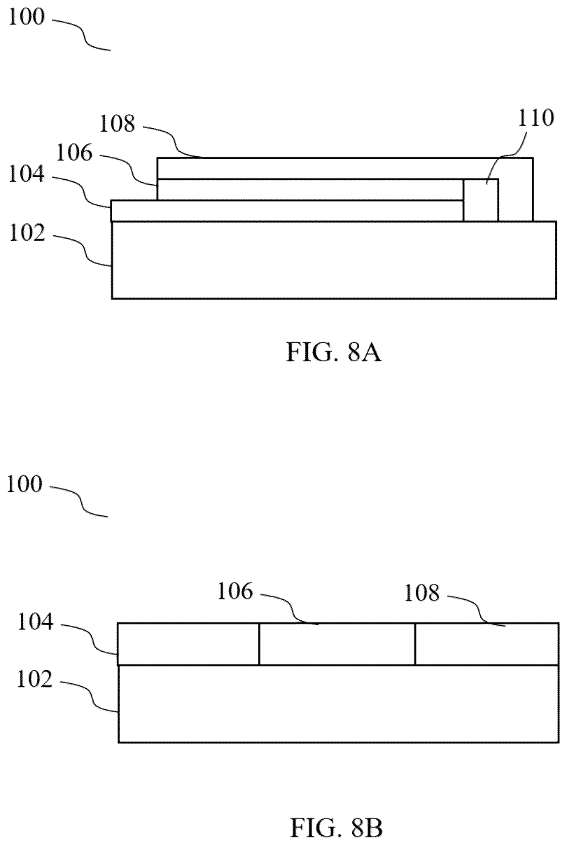

FIG. 8A and FIG. 8B illustrate embodiments of an organic electronic device 100 configured as optically active device. The organic electronic device may include a first electrode 104, e.g. on a substrate 102 or as the substrate; a second electrode 108; and an organic layer 106 arranged such that it is electrically interposed between the first and second electrodes 104, 108. The first and second electrodes 104, 108 may be electrically insulated from each other by an insulating structure 110, e.g. a resin or polyimide. The first and second electrodes 104, 108 may be stacked over each other (FIG. 8A) or may be arranged in a common plane (FIG. 8B).

The organic layer 106 may include the coordination compound according to any of the described or illustrated embodiments, e.g. as a pure compound, in a mixture or in a compound as described before.

In various embodiments, the organic electronic device 100 may be an optoelectronic device, the optoelectronic device being at least one of an organic light emitting diode (OLED), an organic photodetector, or a photovoltaic cell. That is, photons from an external electromagnetic field may be absorbed in the organic layer 106 and converted into current by means of an electrical field between the first and electrodes 104, 108. Such a device would be a photodiode (oPD) and its primarily use may be to sense external light. It would be an organic photovoltaic (OPV) device, if the primarily use is to convert light into current.

In various embodiments the paramagnetic moments of the coordination compounds including divalent Europium may be aligned to exhibit a macroscopic magnetic moment. This macroscopic moment of the organic layer 106 may be employed as part of a magneto-optic or magneto-electric sensor. It may as well be used as part of a touchscreen function of a flat panel display. It may as well be used to build spintronic devices. The alignment of the paramagnetic coordination compound according to various embodiments may be achieved with any suitable technique, for example, but not limited to, techniques that align the coordination compound during, or after processing. For example, the coordination compound may be aligned after processing using a strong external electro-magnetic, or static magnetic field. In conjunction with the application of this external magnetic field, the alignment may be improved or permanently frozen-in by heating the organic layer 106 to be aligned. Hereby the heating may proceed above the glass-transition temperature or beyond the melting temperature of the coordination compound or parts or the whole organic layer 106. Alternatively, the paramagnetic coordination compound may be aligned in-situ during processing of the layer including the paramagnetic coordination compound, for example, but not limited to, by application of a static or dynamic external magnetic field during the processing from either solution or gas phase. In this context, the macroscopic magnetic moment may as well be formed inhomogeneously over the area of the device by applying suitable external magnetic sources during processing.

The organic layer 106 is arranged electrically between the first and second electrodes 104, 108 such that an electronic current may flow from the first electrode 104 through the organic layer 106 to the second electrode 108 and vice versa during operation, e.g. in light emission applications. Alternatively, in photoelectric applications, a charge carrier pair may be formed in the organic layer 106 and charge carriers of the charge carrier pair may be transported to the first and second electrodes 104, 108 respectively. In other words, in light emission applications, upon application of sufficient voltage, holes and electrons are injected from the anode and the cathode, respectively, and drift towards the organic layer 106, where charges of opposite sign recombine to form a short-lived localized excited state. The short-lived excited state may relax to the ground state thereby giving rise to light emission.

The first and second electrodes 104, 108 may be substantially unstructured layers, e.g. for general lighting applications, or may be structured, e.g. for light emitting diodes or transistors for pixels in a display application.

The organic electronic device 100 may be configured to emit substantially monochromatic light such as red, green, blue, or polychromatic light such as white. The light may be emitted through the first electrode 104 (bottom emitter), through the second electrode 108 (top emitter), or through first and second electrodes 104, 108 (bidirectional emitter). The light may as well substantially be emitted in a direction parallel to the organic layer 106 using suitable opaque electrodes 104, 108. In such a layout lasing may be achieved, and the device may be an organic laser, which, in this description, may be considered as a specific type of electroluminescent devices.

The coordination compounds according to various embodiments may have excellent emission properties, including a narrow, deep blue emission spectrum with short excited state lifetime. Given its high atomic weight, the optical transitions of divalent Ytterbium and Europium may be as well widely indifferent to excitation with either spin 1 or spin 0. In other words, they are of phosphorescent type. As such the coordination compounds according to various embodiments may be ideally suited for application in organic electroluminescent devices, such as organic light emitting diodes (OLED). In this description, an electroluminescent device may be any device including an organic layer disposed between and electrically connected to an anode 104/108 and a cathode 108/104. Upon application of sufficient voltage, holes and electrons may be injected from the anode 104/108 and the cathode 108/104, respectively, and drift towards the organic layer 106, where charges of opposite sign recombine to form a short-lived localized excited state. The short-lived excited state may relax to the ground state thereby giving rise to light emission. Relaxation pathways without light emission, such as thermal relaxation, may be possible too, but may be considered undesirable, as they lower the conversion efficiency of current into light of the device.

Further layers may be formed and in electrical connection between the first and second electrodes 104, 108, e.g. configured for charge carrier (electron or hole) injection, configured for charge carrier transport, configured for charge carrier blockage or configured for charge generation. Further optically functional layers, e.g. a further electroluminescent material and/or a wavelength conversion material may be formed electrically between the first and second electrodes 104, 108 and in the optical path of the organic layer 106, e.g. on top of the second electrode 108 and/or on the opposite side of the substrate 102. In addition, encapsulation structure may be formed encapsulating the electrically active area, e.g. the area in which an electrical current flows, and may be configured to reduce or avoid an intrusion of oxygen and/or water into the electrically active area. Further optically functional layers, e.g. an antireflection coating, a waveguide structure and/or an optical decoupling layer may be formed within the optical light path of the organic layer 106.

As example, hole or electron blocking layers may be used to optimize the individual hole and electron currents through the organic electronic device 100. This may be known to those skilled in the art as charge balance in order to optimize efficiency and operational stability. In various embodiments, dedicated hole or electron charge transport layers may be present in the organic electronic device 100 to space the emission region from the first and second electrodes 104, 108.

Examples of hole transport materials include known materials such as fluorene and derivatives thereof, aromatic amine and derivatives thereof, carbazole derivatives, and polyparaphenylene derivatives. Examples of electron transport materials include oxadiazole derivatives, triazine derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and its derivative, diphenoquinone derivatives, and metal complexes of 8-hydroxyquinoline and its derivatives.

In various embodiments, those charge transport layers may include electrical dopant molecules or metals, or may be in contact to charge injection layers.

Any of those auxiliary layers may be fully organic or may include inorganic functional moieties. For example, charge transport layers may be made of the class of Perovskite materials.

The coordination compound as illustrated or described in any one of the embodiments may be used as a pure organic emitting layer 106 of any thickness in the range of 1 nm and 100 nm.

The organic layer 106 may in various embodiments include charge transport materials to improve charge transport into and through the organic layer 106. Charge transport materials may be any material that is able to transport either holes or electrons or both types of charges. In particular a charge transport material may be any aryl, or heteroaryl organic compound or any metal complex or any mixture thereof. The volume percentage of the coordination compound as a function of the combined charge transport materials may be between 0.5 and 99.5 vol % in the organic layer 106.

In various embodiments, the oxidation potential of the coordination compound of the organic layer 106 may be higher compared to all charge transport materials present in the organic layer 106. A wide variety of techniques on how to measure oxidation potentials have been published in the literature. However, for the purpose of various embodiments, the particular technique of how to measure the oxidation potential is not essential, e.g. only the relative order between the coordination compound and the charge transport materials may be of importance. Oxidation potentials may for example be deducted using quantum mechanical computing techniques based on density functional theory and experimental techniques are very well known in the art, e.g. see R. J. Cox, Photographic Sensitivity, Academic Press, 1973, Chapter 15.

The organic layer 106 may contain any other organic or inorganic material in a range of 0.1 to 99.9 vol % that are not intended to transport charges. For example. The organic layer 106 may include polymers (in a mixture or as a compound) may be added to improve film quality and prevent crystallization. Other materials may be added to evenly space the coordination compound inside the organic layer 106.

In color conversion materials, the divalent lanthanides according to various embodiments may allow the ease of processing using vacuum based techniques. Thus, an application of divalent Europium or Ytterbium in organic electronic devices, such as organic photovoltaic (OPV), organic light emitting diodes (OLED), organic sensors, organic memory or organic sensors may be of advantage. In other words, the evaporation temperature of the compounds including the divalent lanthanides may be sufficiently low to allow for thermal vacuum processing techniques to be used. Reduced evaporation temperatures can be achieved by converting the inorganic salts including the metal in divalent oxidation state into metal-organic coordination compounds. In such an environment the ionic character of the compound is strongly suppressed, if compared to a similar inorganic salt. Consequently, the evaporation temperature is reduced and incorporation into organic electronic devices becomes possible using state-of-the-art vacuum deposition techniques.

In contrast, in the related art, macrocycles have not been used to enable divalent lanthanides as OLED emitting materials. In particular, none of the conventional, hydrogen substituted macrocycles are able to sufficiently stabilize divalent lanthanides in order to allow for OLED fabrication, let alone stable OLED emission. On the other hand, poly non-hydrogen substituted macrocycles may provide the needed chemical stability for divalent lanthanides to process them into OLED and to provide light emission of high efficiency and deep blue color.

The excitation of the light emitting coordination compound may be electrically confined. This way, high efficiencies may be achieved. Thus, confinement layers may be formed adjacent to the organic layer 106, wherein the confinement layers may have a triplet energy Ti higher than 1.8 eV, e.g. higher than 2.3 eV, e.g. higher than 2.7 eV. Similarly, any material of the organic layer 106 (other than the electroluminescent compound) may have a triplet energy Ti higher than 1.8 eV, e.g. higher than 2.3 eV, e.g. higher than 2.7 eV.

In various embodiments, the organic electronic device 100 includes two or more sub units each including at least one light emitting layer. The subunits may be stacked over each other physically separated and electrically connected by a charge generation layer or, alternatively, may be arranged side by side. The subunits may be subpixels of a pixel in a display or general lighting application. The light emitted by the subunits may be mixed to generate a light of a predetermined color. Each subunit may emit light of the same or a different color. The overall light emitted by such organic electronic device 100 may contain a narrow spectral region, such as blue, or may contain a wide spectral region such as white, or a combination thereof. The coordination compound illustrated or described in any one of the embodiments may or may not be present in any subunit of the organic electronic device 100.

In various embodiments, the light emitted by the organic electronic device 100 may be in optical contact to at least one optically active layer, including any optically active materials such as organic molecules or quantum dots. The optically active layer may be a spectral filter element, which may absorb part of the light emitted by the organic electronic device 100. In another embodiment, the optically active layer may absorb at least part of the light emitted by the organic electronic device 100 and may reemit it at longer wavelength (wavelength conversion).

As example, the organic layer 106 may be configured to emit light substantially at wavelengths shorter than 500 nm and the optically active layer may be configured to substantially reemit light at wavelengths longer than 500 nm. The optically active layer may be placed in between the anode and cathode of the organic electronic device 100 or outside of it. The optically active layer may as well be part of the organic layer 106.

The organic electronic device 100 may be configured as a large area OLED device used for illumination, signage, or as a backlight. Alternatively, the organic electronic device 100 may include a plurality of OLEDs arranged in a pixilated layout (plurality of OLED pixels) that are individually connected electrically, e.g. for flat panel display applications. Here, individual pixels may have the capability of emitting light of substantially narrow spectral portions; especially of red, green, and blue. The coordination compound may or may not be present in any of the individual pixels.

In another embodiment, the individual pixels may be configured to emit white light. Red, green, and blue spectral portions are generated by using suitable filter elements in optical contact with the pixelated OLEDs.

In another embodiment, the OLED pixels emit blue light and the red and green spectral portions may be generated by using a suitable color conversion element in optical contact with the OLED pixels.

As example, the organic electronic device 100 may include in various embodiments an anode 104/108, a cathode 108/104, and the organic layer 106 disposed between the anode and the cathode. The organic layer 106 includes the metal organic coordination compound according to a described or illustrated embodiment. The metal organic coordination compound includes a macrocyclic organic ligand that coordinates to a lanthanide in its divalent oxidation state.

In various embodiments, the organic electronic device 100 includes an anode 104; a cathode 108; and an organic layer 106 disposed between the anode 104 and the cathode 108. The organic layer 106 includes an electroluminescent coordination compound according to various embodiments. The coordination compound includes at least one divalent lanthanide coordinated by a cyclic organic ligand according to formula 1 illustrated in FIG. 1A. Here, i may be larger than 3; and n may be equal to 1, 2, or 3; and L for each occurrence may be independently selected from arylenes or fragments

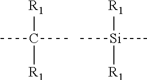

And X may be independently selected for each occurrence from:

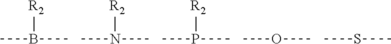

Further, R₁ to R₂ may be any covalently bound substituents being identical or different in each occurrence of n and i. In various embodiments, n and i may be connected to each other thereby forming a polycyclic ligand. Further, R₁ to R₂ may be at least in 3 occurrences not hydrogen. The divalent Lanthanide may be Europium or Ytterbium. The organic ligand according to the formula illustrated in FIG. 1A may be electrically neutral. The coordination compound may include at least one negatively charged anion, which may not be covalently bound to the organic ligand. The negatively charged anion may include more than one atom. The coordination compound may be imbedded into at least one second electrically neutral organic compound. The second organic compound may have a triplet energy higher than 2.5 eV. The coordination compound may have a higher hole affinity compared to the second organic compound.

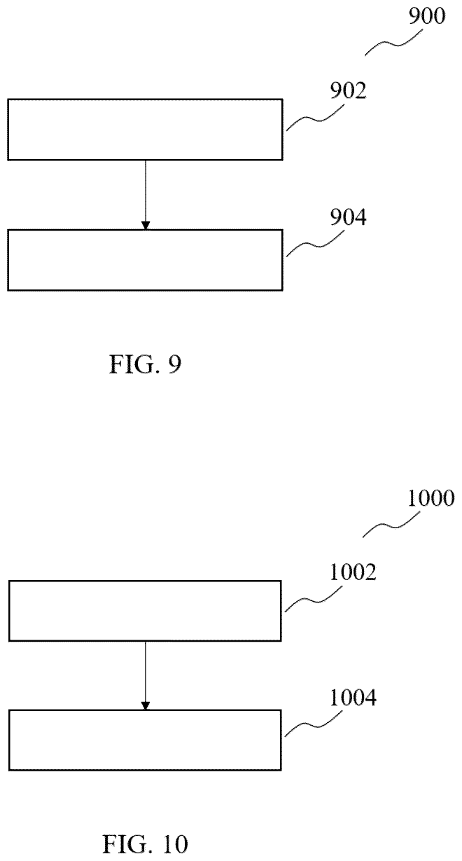

FIG. 9 illustrates a flow diagram of a method of forming an organic device, the method may include forming 900 of a layer of the coordination compound according to any of the described or illustrated embodiments as a pure compound or in a mixture or linked to a polymer as described before. The layer may be deposited from a gas phase, in particular using an evaporation and/or sublimation process, and/or by a solution-based process. The forming 900 of the layer may include forming 902 a first layer including the organic ligand and forming 904 a second layer directly in contact with the first layer, wherein the second layer includes a divalent lanthanide salt. The second layer may be deposited by a solution-based or thermal vacuum based process.

An organic electronic device 100 according to various embodiments may be fabricated using a wide range of commonly used techniques, including, but not limited to, deposition of all or some layer, from gas phase vacuum deposition, solution phase, or gas phase using a carrier gas method.

In various embodiments, deposition via the gas phase in vacuum may be used, whereby the coordination compound may either undergo sublimation or evaporation. The transfer into the gas phase may be improved by using a carrier gas technology, whereby an inert gas that may not be deposited into the organic layer may be helping the sublimation or evaporation of the coordination compound to be deposited. During the deposition process from gas phase, the coordination compound may as well be co-deposited with one or more material to fabricate any desired mixed layers.

In one embodiment, the coordination compound according to various embodiments may be formed in-situ using gas phase deposition techniques. Here, the organic ligand of the formula illustrated in FIG. 1A excluding the divalent lanthanide may be first (902) deposited onto a suitable substrate or other organic layer thereby forming a seed layer. Any suitable technique may be used to fabricate this seed layer. This seed layer including the organic ligand may include any other material; preferred may be organic charge transport materials, for example, suitable to achieve hole transport. Preferred may be in various embodiments inert organic or inorganic materials that aid the layer formation, improve its thermal stability, or improve the distribution of the organic ligand within this seed layer. In the next deposition step (904) and sequential to forming (902) the seed layer including the organic ligand according to the formula illustrated in FIG. 1A but excluding the divalent lanthanide metal, a molecular salt including a divalent lanthanide may be evaporated. The divalent lanthanide salt may be any charge neutral compound including a lanthanide in its divalent oxidation state and one or two suitable anions. Preferred may be two single, negatively charged, inorganic anions such as compound a1 to compound a41 (see FIG. 5). The divalent lanthanide salt may as well be simultaneously co-deposited with one or more material to fabricate mixed layers. At the interface of the seed layer including the organic ligand, according to the formula illustrated in FIG. 1A, and because of its high thermal activation energy, the divalent lanthanide salt may interact with the organic ligand to form the coordination compound according to various embodiments in-situ. Alternatively, to use a molecular salt, a lanthanide metal vapor may be used to form the coordination compound according to various embodiments in-situ. In the latter case, an oxidation of the lanthanide takes place. This reaction may be improved by the presence of suitable electron acceptor units in the seed layer. Suitable electron acceptors present in the seed layer may be the reduced, i.e. charge neutral, versions of compounds a30 to a42 (FIG. 5), also known as p-dopants.

Another preferred technique to fabricate layers including the coordination compound according to various embodiments may be deposition from a liquid phase using a mixture or a single organic solvent, whereby the coordination compound according to various embodiments may be dissolved or forms a suspension within the organic solvent; in this description may be referred to as the ink. The ink using this deposition process, may include a wide variety of other materials apart from the coordination compound according to various embodiments to allow fabrication of mixed layers from solution. Additives within the ink may for example, but may not be limited to, be organic or inorganic materials capable of transporting charges, materials that improve the film formation, materials that improve the distribution of the coordination compound within a host material, organic or inorganic materials that improve the efficiency of the device, e.g. by reducing the refractive index. The deposition from solution may not be limited to any specific technique. Examples of the deposition from solution include spin coating, casting, dip coating, gravure coating, bar coating, roll coating, spray coating, screen printing, flexographic printing, offset printing, inkjet printing.

Various post processing techniques may be applied to improve the performance or stability of the organic electronic device. In one embodiment, some or all layers of the organic electronic device include functional groups capable of chemically crosslinking upon thermal or optical excitation thereby forming larger covalently bound molecules with improved physical properties. In a special case of this embodiment, the crosslinking takes place during applied electrical field, especially such that anion drift, i.e. light emitting cell behavior, may be permanently frozen-in after the crosslinking has taken place.

Many of the coordination compounds according to various embodiments exhibit a high paramagnetic moment, e.g. in case the divalent lanthanide is Europium. The paramagnetic moments of the coordination compound according to various embodiments may be aligned to exhibit a macroscopic magnetic moment. This macroscopic moment of the (organic) layer (106) may be employed as part of a magneto-optic or magneto-electric sensor integrated into the organic electronic device of the invention. It may as well be used as part of a touchscreen function of an organic electronic device flat panel display. It may as well be used to build opto-electronic light emitting spintronic devices.

The alignment of the paramagnetic coordination compound according to various embodiments may be achieved with any suitable technique, for example, but not limited to, techniques that align the coordination compound during, or after processing. For example, the coordination compound may be aligned after processing using a strong external electro-magnetic, or static magnetic field. In conjunction with the application of this external magnetic field, the alignment may be improved or permanently frozen-in by heating the organic layer to be aligned. Hereby the heating may proceed above the glass-transition temperature or beyond the melting temperature of the coordination compound or parts or the whole organic layer (106).

Alternatively, the paramagnetic coordination compound may be aligned in-situ during processing of the layer including the paramagnetic coordination compound, for example, but not limited to, by application of a static or dynamic external magnetic field during the processing from either solution or gas phase. In this context, the macroscopic magnetic moment may as well be formed inhomogeneously over the area of the organic electronic device by applying suitable external magnetic sources during processing.

FIG. 10 illustrates a flow diagram of a method to synthesize the coordination compound according to any of the described or illustrated embodiments. The method 1000 may include a reaction of a divalent lanthanide salt and an organic ligand according to any one of FIG. 1 to FIG. 5 at pressure greater than or equal to about 1 bar. The method 1000 may include that the divalent lanthanide salt is coordinated with an organic precursor 1002 and subsequently at least one of the groups R₁, R₂, R₃, R₄, R₅, R₆ is attached to the organic precursor 1004.

In various embodiments, the coordination compound may be formed in-situ using deposition from solution. Here, the organic ligand of the formula illustrated in FIG. 1A, excluding the lanthanide metal, may be first deposited onto a suitable substrate or other organic layer thereby forming a seed layer. Any suitable technique may be used to fabricate this seed layer. This seed layer including the organic ligand may include any other material. Preferred may be organic charge transport materials, for example suitable to achieve hole transport for organic electronic devices. Preferred may be further additional inert organic or inorganic materials that aid the layer formation or improve its thermal stability or improve the distribution of the organic ligand within this seed layer. In a next deposition step and sequential to forming the seed layer including the organic ligand according to the formula illustrated in FIG. 1A excluding the metal, a layer including a molecular salt including a divalent lanthanide may be fabricated using a solution process. The divalent lanthanide salt may be any charge neutral compound including a lanthanide in an oxidation state 2+ and one or two suitable anions. The anions may as well be covalently bound to a suitable polymer. Suitable anions may be for example, but not limited to, compound a1 to compound a19 and fluorinated fullerenes. The ink including the divalent lanthanide salt may as well include one or more additive to fabricate mixed layers. These additives may be organic or inorganic materials to aid charge transport, may be organic or inorganic materials that improve the efficiency of the device, or may be any material that improves the film formation. At the interface of the seed layer including the organic ligand according to the formula illustrated in FIG. 1A, the solubilized divalent lanthanide metal will interact with the organic ligand to form the coordination compound according to various embodiments in-situ.

In various embodiments, the method of forming an organic electronic device includes forming an anode; forming a cathode; and forming an organic layer disposed between the anode and the cathode. The organic layer may be formed to include an electroluminescent coordination compound. The coordination compound may include at least one divalent lanthanide coordinated by a cyclic organic ligand according to formula 1 illustrated in FIG. 1A. Here, i may be larger than 3; and n may be equal to 1, 2, or 3; and L for each occurrence may be independently selected from arylenes or fragments

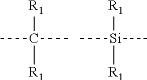

And X may be independently selected for each occurrence from:

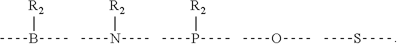

Further, R₁ to R₂ may be any covalently bound substituents being identical or different in each occurrence of n and i. In various embodiments, n and i may be connected to each other thereby forming a polycyclic ligand. R₁ to R₂ may be at least in 3 occurrences not hydrogen. Further, the coordination compound may be deposited from gas phase. Alternatively, the coordination compound may be deposited using a solution-based process

The divalent Lanthanide may be Europium or Ytterbium. The coordination compound may be formed by a co-deposition from the gas phase of a divalent lanthanide salt and the organic ligand. The coordination compound may be formed by first depositing the organic ligand and second depositing a divalent lanthanide salt from the gas phase. The organic ligand according to the formula illustrated in FIG. 1A may be formed to be electrically neutral.

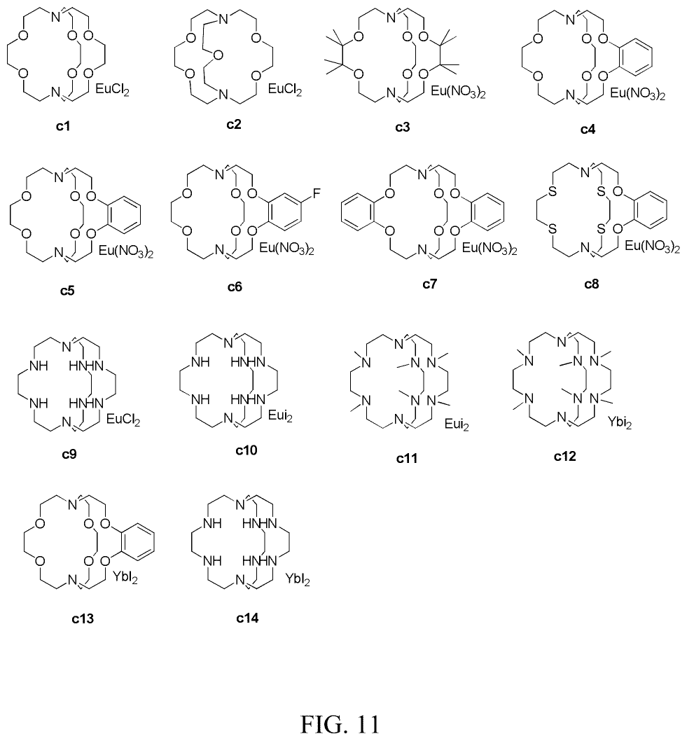

As such, several compounds including Eu²⁺ (c1-c11 illustrated in FIG. 11) and Yb²⁺ (c12-c14 illustrated in FIG. 11B) may be considered as comparative examples. In contrast, in the coordination compounds according to various embodiments, substituents larger than —CH3 group may be attached to the nitrogen atoms of an azo-cryptand. This way, a needed chemical stability to Eu²⁺ and Yb²⁺ coordination compounds is provided. The application of such coordination compounds may be useful for an improvement of a luminescence efficiency and a lifetime of the electroluminescent device.

a wide range of possibilities to synthesize the coordination compounds according to various embodiments. Some qualitative schemes are briefly discussed below for the purpose of illustration only.

In one qualitative scheme, the organic ligand according to the formula illustrated in FIG. 1 and FIG. 3 may be synthesized first. In a next step a suitable salt including the divalent Europium or Ytterbium may be added. By choosing suitable conditions, the coordination compound according to various embodiments may be formed and may precipitate. Owning to their bulky ligands, the coordination compounds according to various embodiments may exhibit a very high kinetic barrier against reaction of the metal cation with the environment. In such situation, the reaction of the organic ligand with the divalent lanthanide metal salt may result in poor yields. Therefore, in a reaction scheme according to various embodiments, the reaction of the organic ligand with the divalent lanthanide metal salt may be carried out in an environment capable of sustaining elevated temperatures and pressures, such as an autoclave. Preferred reaction conditions include elevated temperatures above 293K, e.g. at temperatures above 333K, e.g. at temperatures above 373K and pressures higher than 1 bar, e.g. higher than 1.5 bar, e.g. pressures higher than 5 bar, e.g. pressures higher than 20 bar.

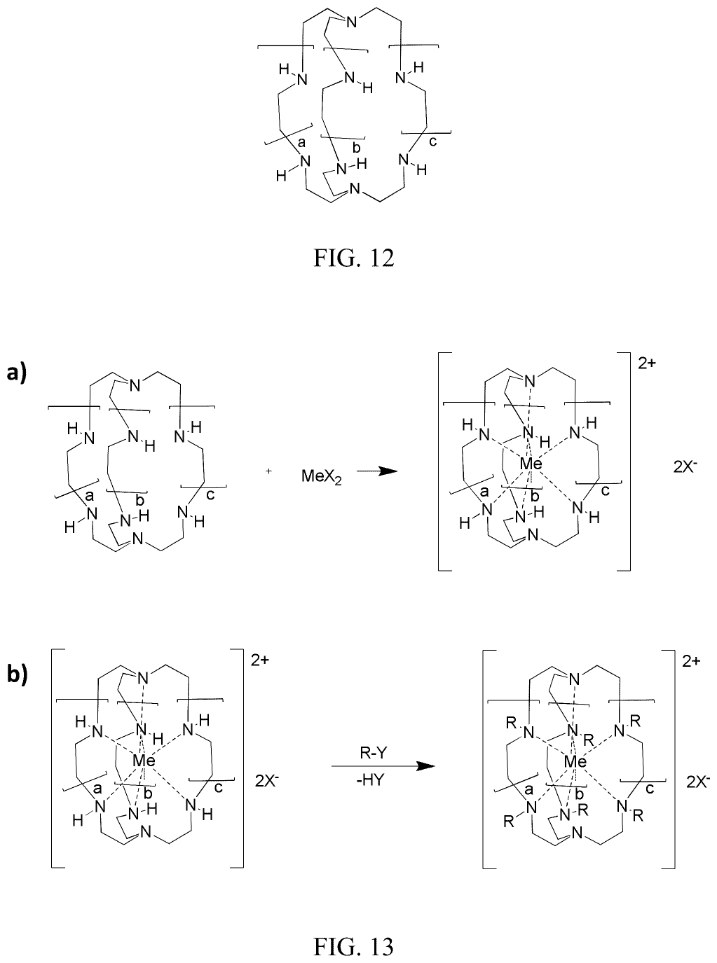

In an alternative qualitative reaction scheme, the divalent lanthanide may be first coordinated by a precursor ligand and in one or more subsequent synthesis steps at least one side chain of the type R₁ to R₆ may be attached to yield a coordination compound according to various embodiments. The precursor may be any organic ligand suitable to coordinate divalent lanthanides and suitable to be reacted to form the final product according to various embodiments. Preferred may be cryptate type ligands. Even more preferred may be ligands according to the formula illustrated in FIG. 12. Here, a, b and c may be independently an integer of 0 or more.

In another embodiment, the synthesis of coordination compound could be done according to the schema 1 illustrated in FIG. 13. Here, Me may be Eu²⁺ or Yb²⁺ and a dashed line may represent the coordination bond of the ligand to the central metal ion; a, b, c may be integer of 0 or more, X—may represent the single charged anion, R—Y may represent the compound, which may be able to react with secondary amines. For example, compounds c27, c33 (FIG. 6) could be prepared according to schema 1,

The (electroluminescent) coordination compound according to various embodiments may thus be extremely stable and as such ideally suited for a large variety of processing methods and applications.

For one or more aspects, at least one of the components set forth in one or more of the preceding FIGS. may be configured to perform one or more operations, techniques, processes, and/or methods as set forth in the example section below.

### EXAMPLES

The examples set forth herein are illustrative and not exhaustive.

Example 1 is an electroluminescent coordination compound, wherein the coordination compound includes at least one divalent lanthanide coordinated by a cyclic organic ligand according to formula 1:

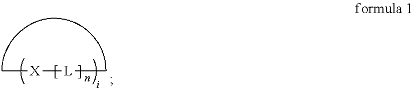

wherein i is larger than 3; and n is equal to 1, 2, or 3; and L for each occurrence is independently selected from arylenes or biradical fragments of

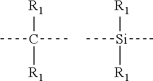

and X is independently selected for each occurrence from the group of:

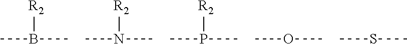

wherein R₁ and R₂ are any covalently bound substituents being identical or different in each occurrence of n and i; and wherein R₁ and/or R₂ are at least in 3 occurrences not hydrogen.

In Example 2, the subject matter of Example 1 can in various embodiments include that the divalent Lanthanide is Europium or Ytterbium.

In Example 3, the subject matter of Example 1 can in various embodiments include that R₁ and R₂ are connected to each other thereby forming a polycyclic ligand, wherein at least two R₂ are connected forming a bridge to form the polycyclic ligand.

In Example 4, the subject matter of any one of Examples 1 to 3 can in various embodiments include that the organic ligand according to formula (1) is electrically neutral.

In Example 5, the subject matter of any one of Examples 1 to 4 can in various embodiments include that the coordination compound includes at least one negatively charged anion, which is not covalently bound to the organic ligand.

In Example 6, the subject matter of Example 5 can in various embodiments include that the negatively charged anion includes more than one atom.

In Example 7, the subject matter of any one of Examples 2 to 6 can in various embodiments include that the cyclic organic ligand of formula 1 has a structure according to formula 2:

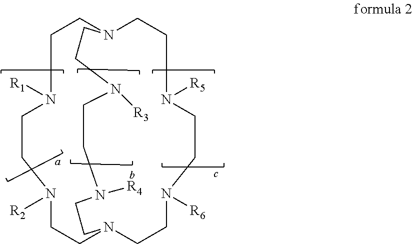

wherein R₁, R₂, R₃, R₄, R₅, R₆ independently in each occurrence represent an organyl group, including at least one carbon atom and at least one additional atom; whereby the additional atom is not a hydrogen or not an organoheteryl group, and a, b, c are each independently an integer of 0 or more.

In Example 8, the subject matter of Example 8 can in various embodiments include that a, b and c are each equal to 1.

In Example 9, the subject matter of any one of Examples 7 or 8 can in various embodiments include that R₁, R₂, R₃, R₄, R₅, R₆ are in each occurrence independently selected from the group of f1 to f78, wherein f1 to f78 are:

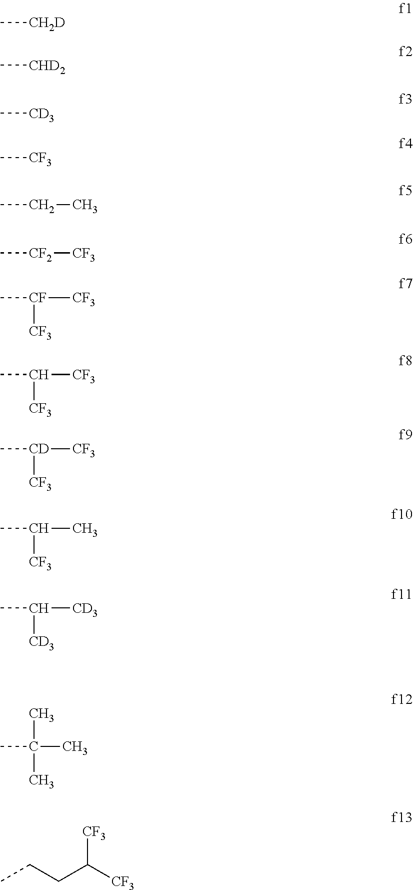

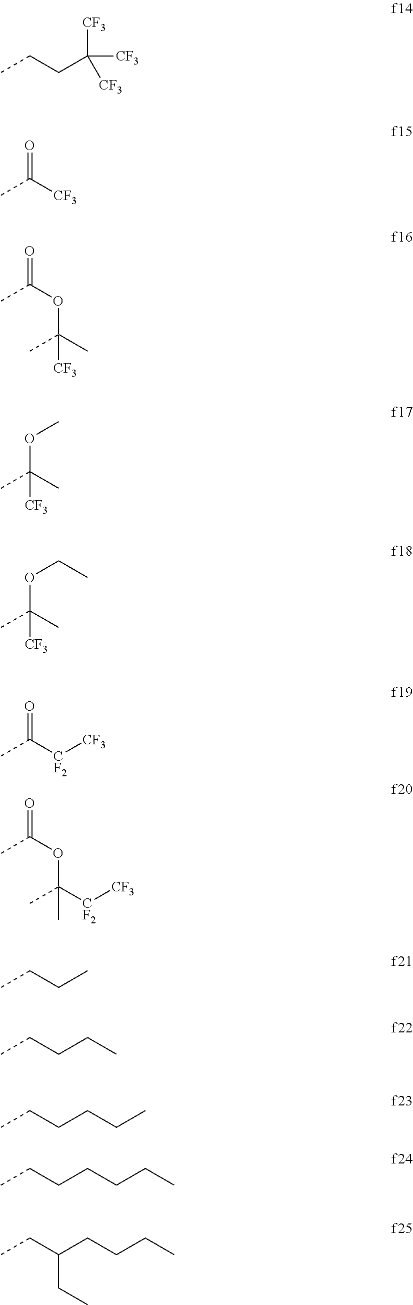

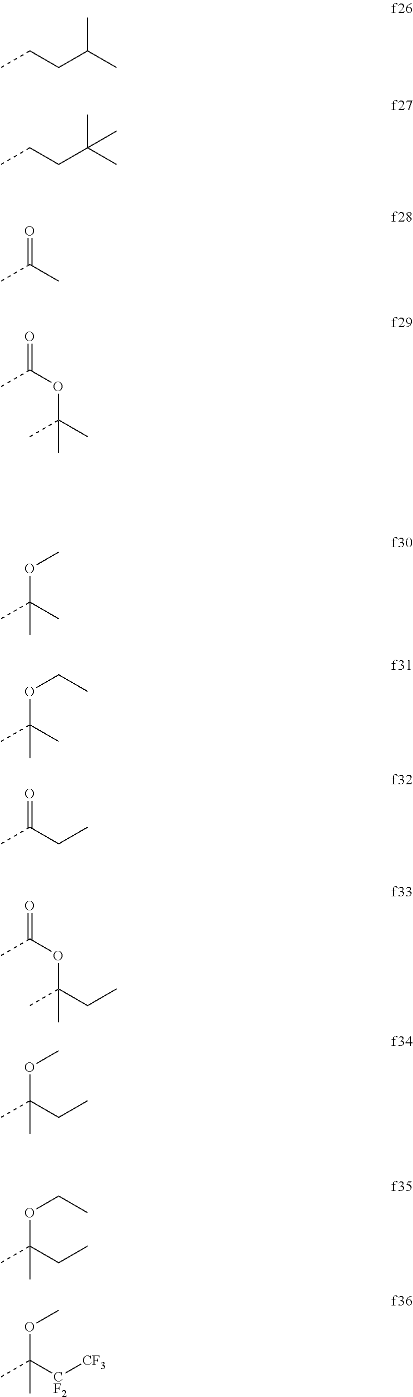

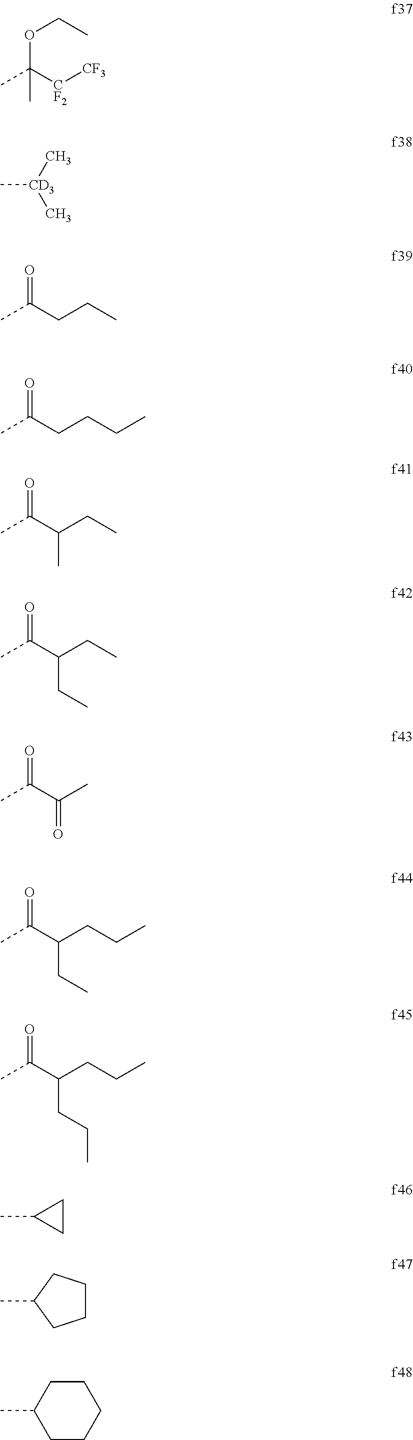

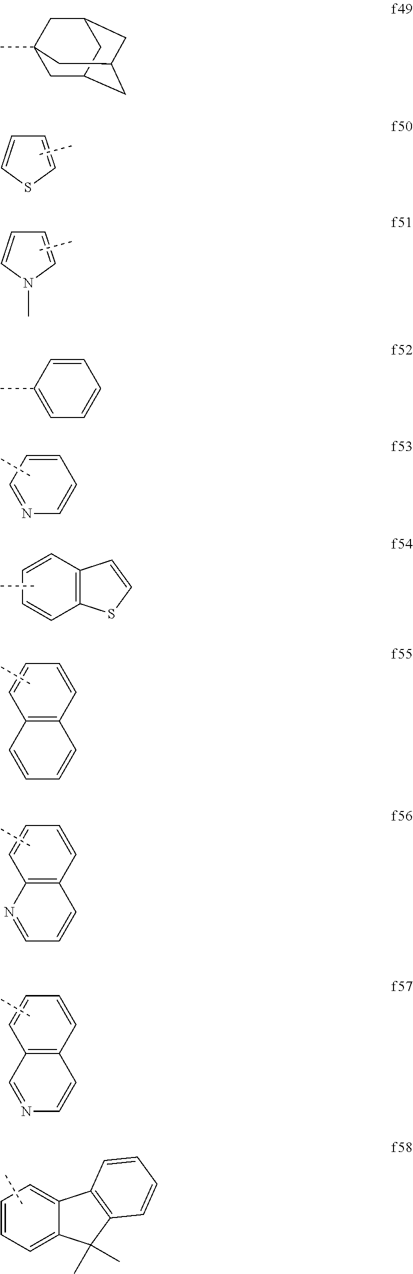

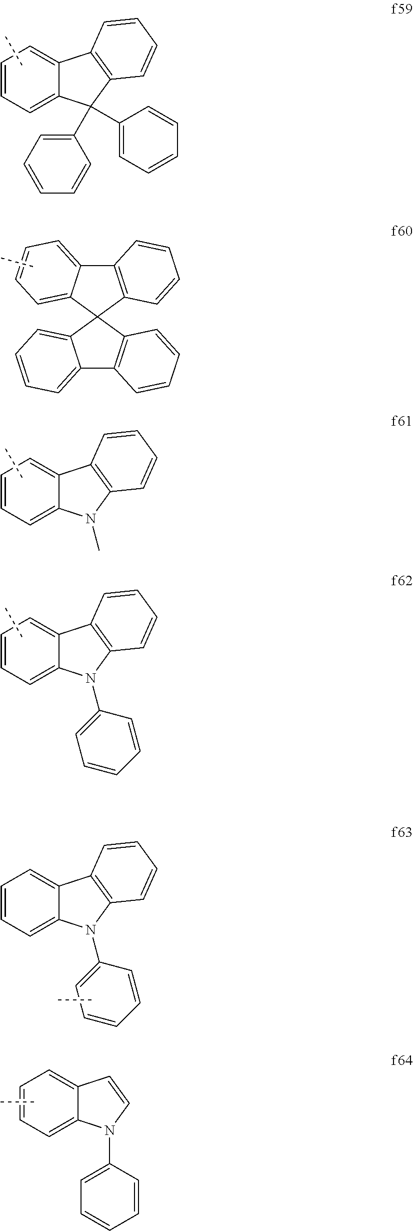

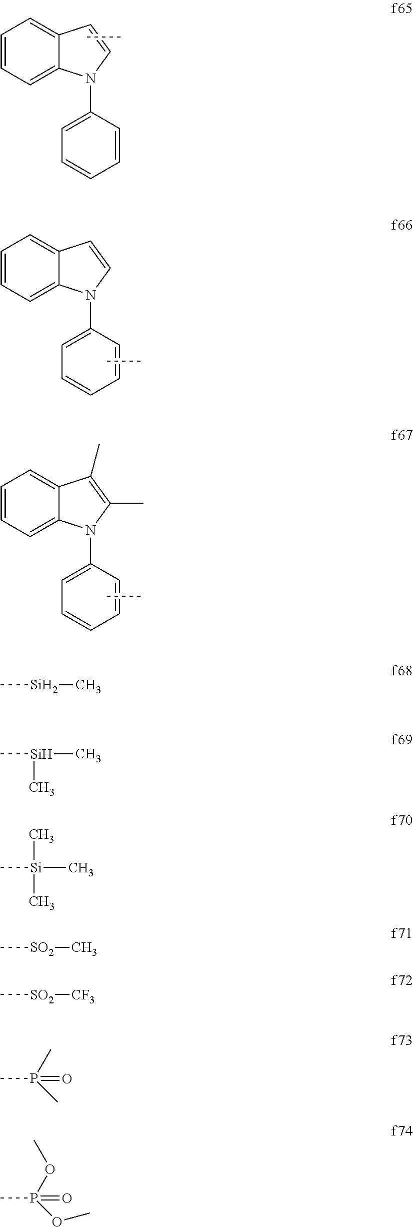

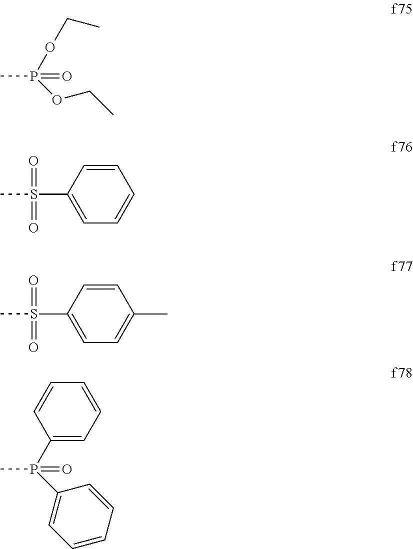

wherein a dash line represents the preferred connection point.

In Example 10, the subject matter of any one of Examples 7 or 9 can in various embodiments include that the coordination compound includes at least one negatively charged anion, which is not covalently bound to the organic ligand, wherein the negatively charged anion is at least one selected from the group of a1 to a42, wherein a1 to a42 are:

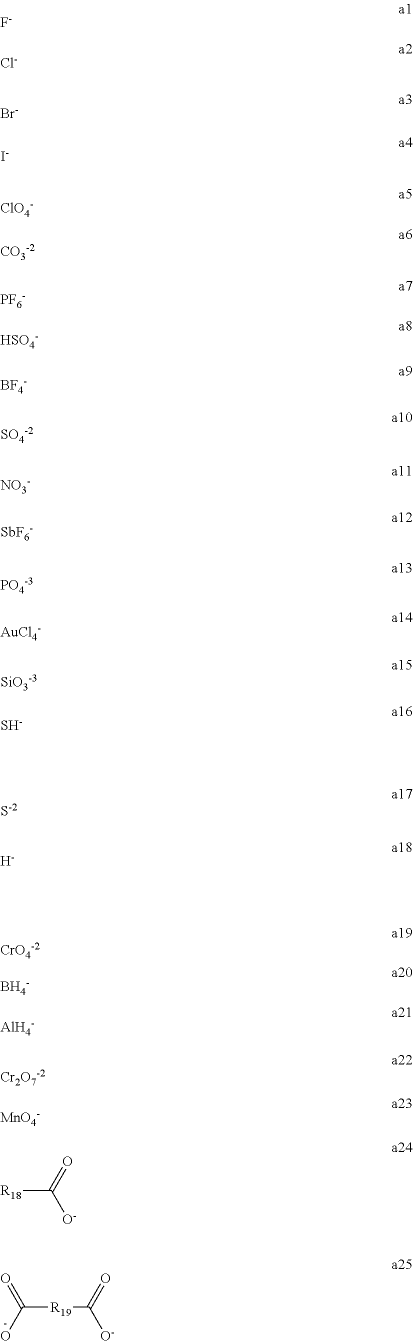

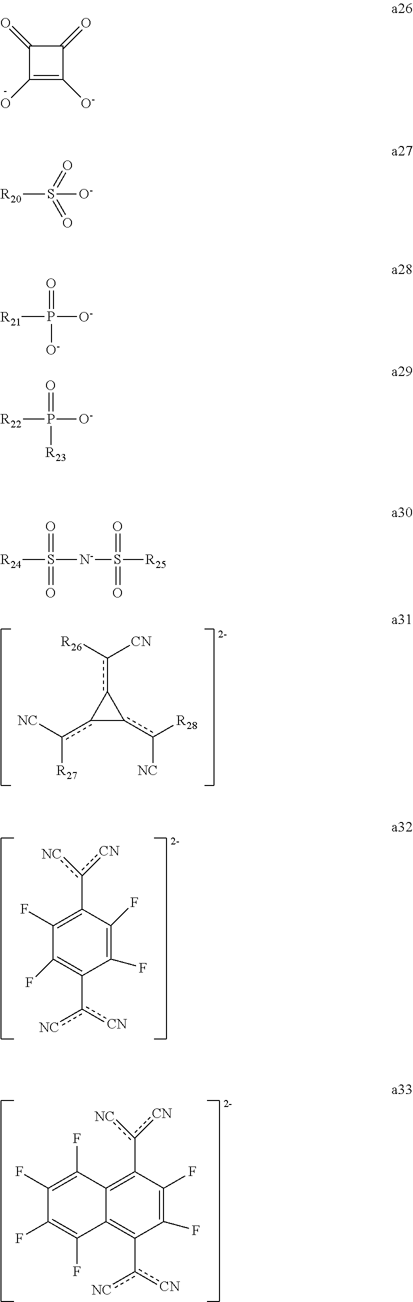

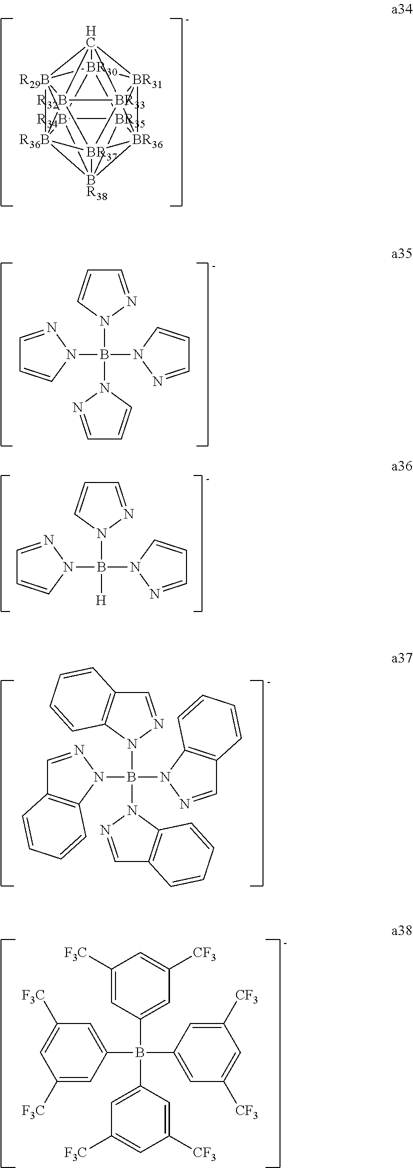

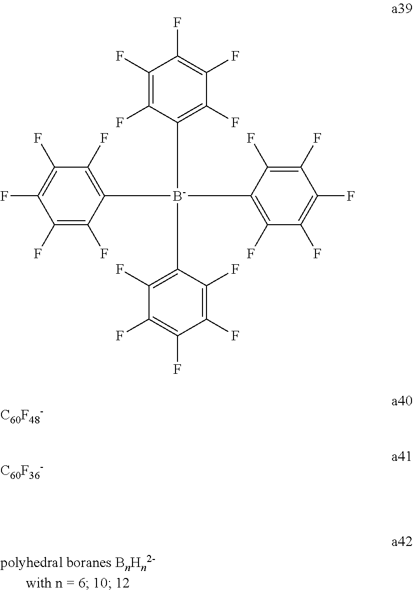 [SMILES: Fc1c(F)c(F)c([B-](c2c(F)c(F)c(F)c(F)c2F)(c2c(F)c(F)c(F)c(F)c2F)c2c(F)c(F)c(F)c(F)c2F)c(F)c1F ; "C60F36-" ; "C60F48-"]

In Example 11, the subject matter of any one of Examples 1 to 10 can in various embodiments include that the coordination compound is selected from the group of c15 to c44, wherein c15 to c44 are:

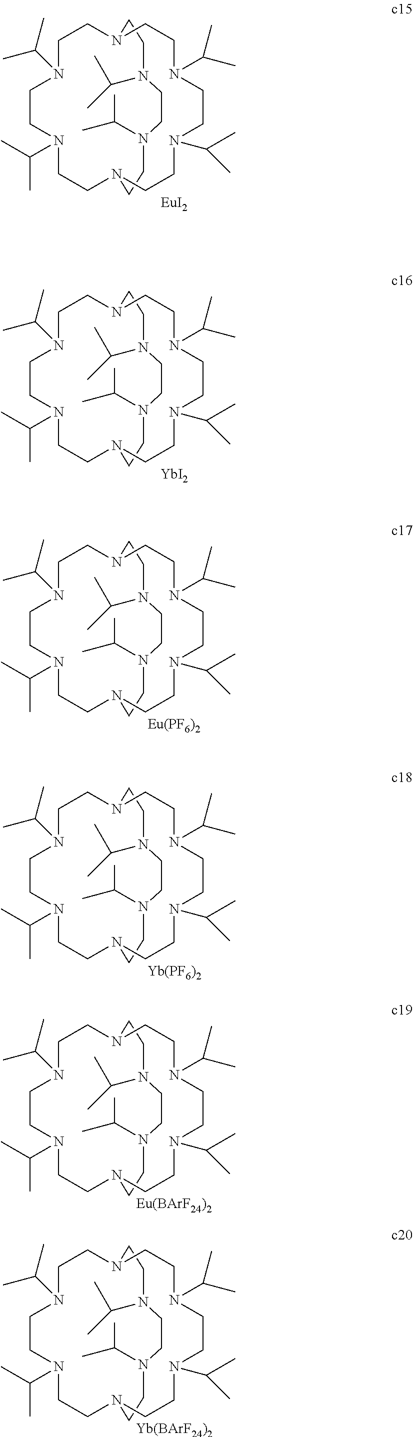

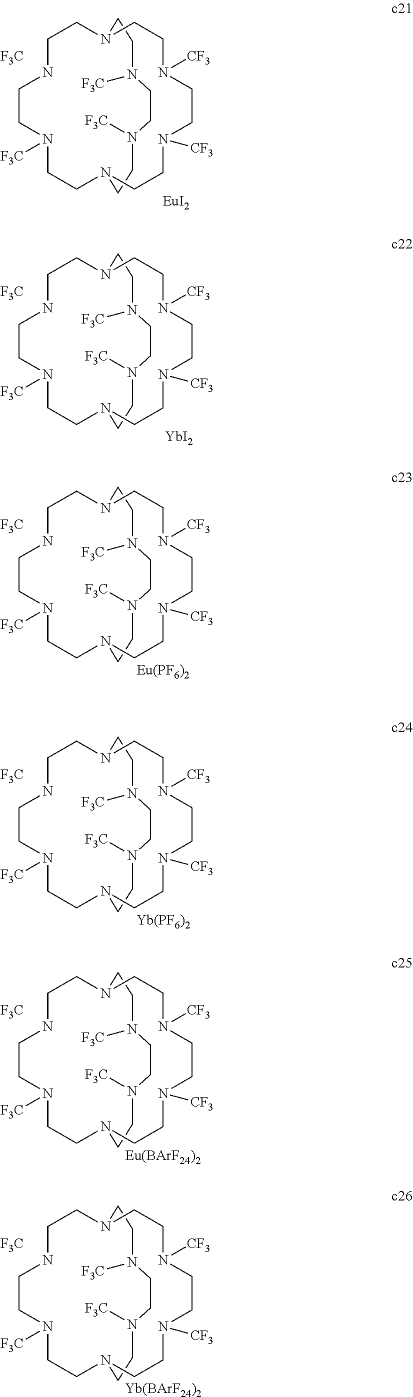

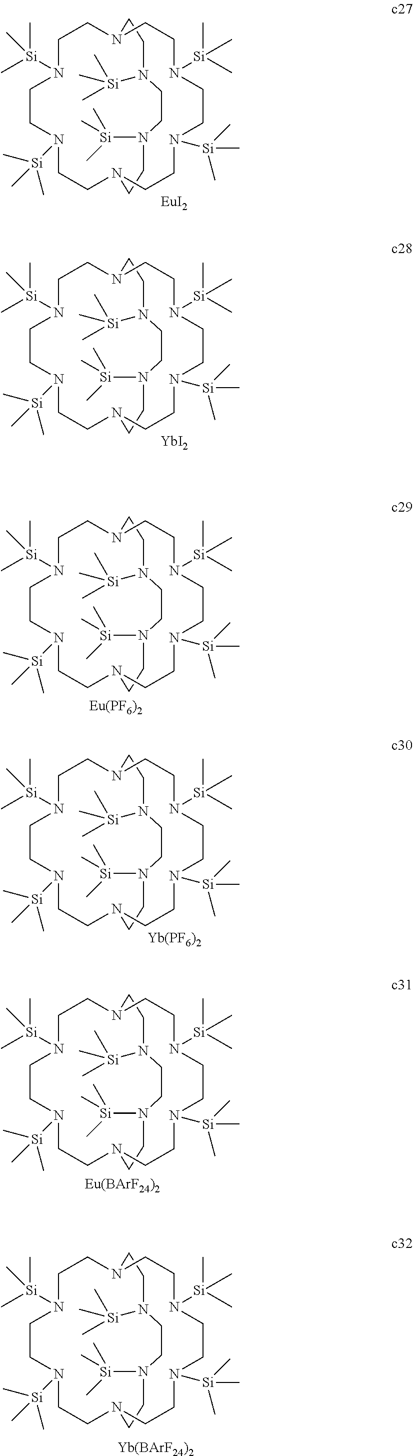

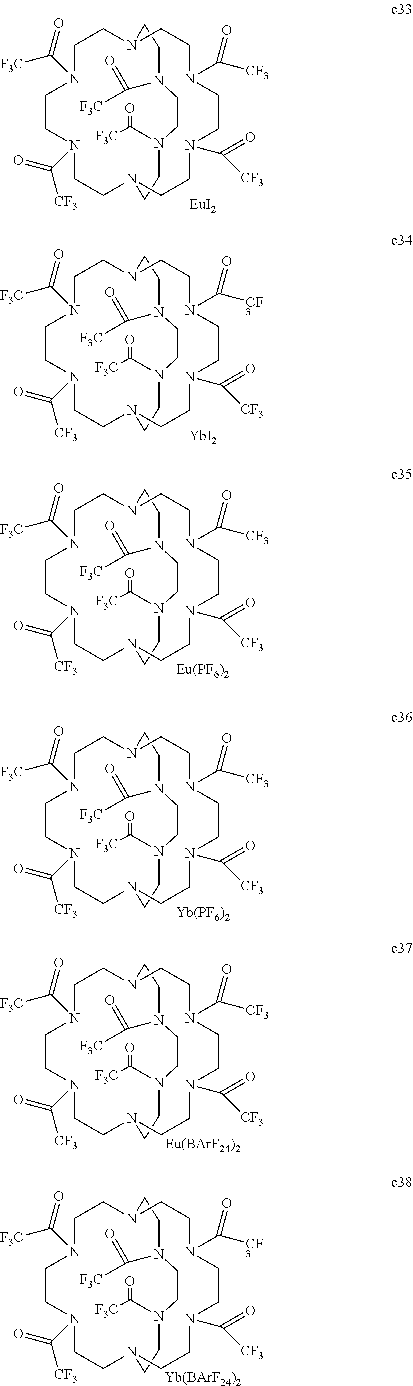

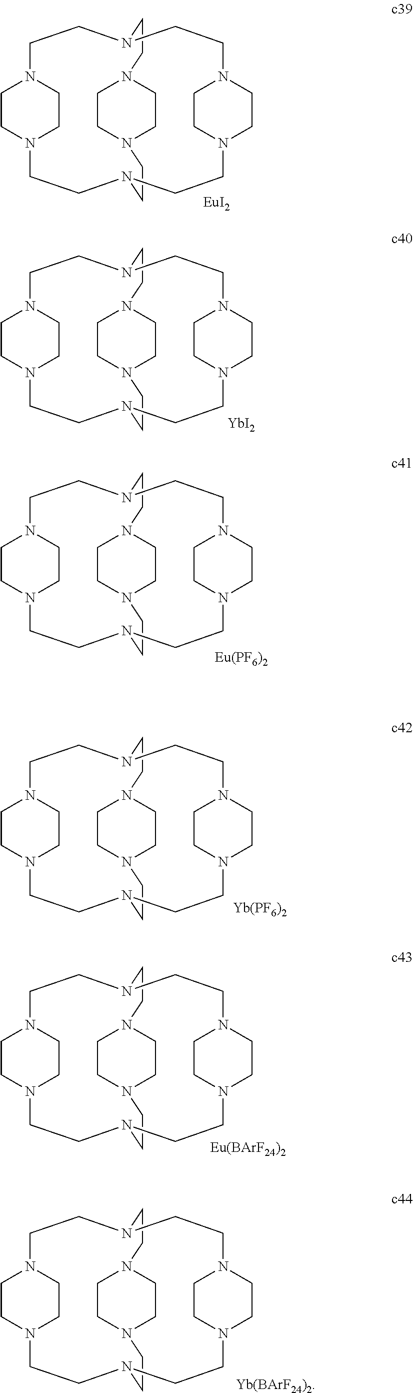

and wherein BArF₂₄ represent the anion a38.

A In Example 11a, the coordination compound comprises at least one negatively charged anion, which is not covalently bound to the organic ligand, wherein the negatively charged anion is an organoboron compound containing at least three substituted or unsubstituted cyclic or heterocyclic organic groups covalently bound to the boron, or is at least one selected from the group of a1 to a18, wherein a1 to a18 are:

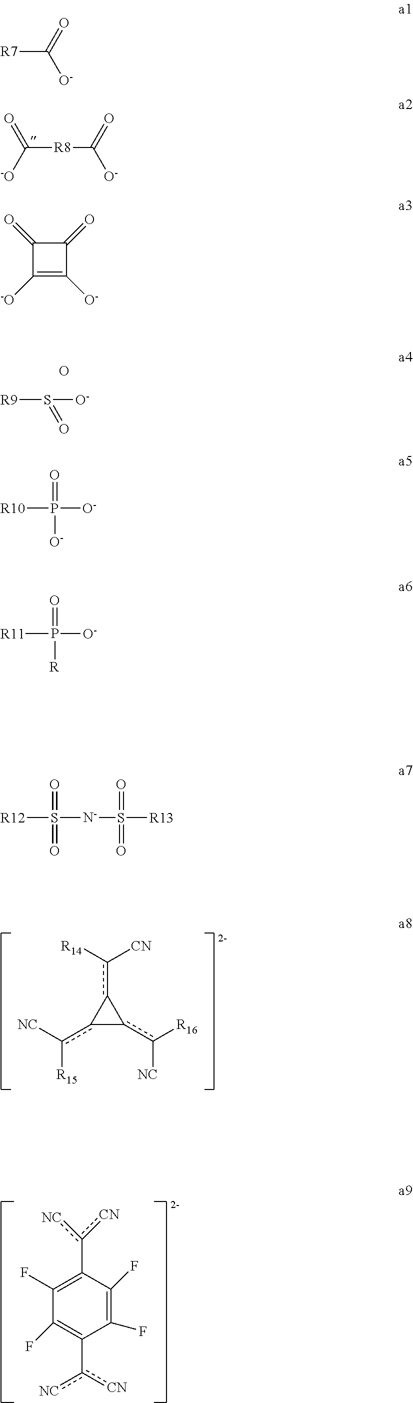

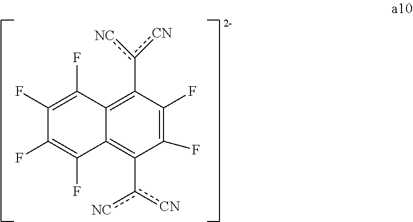

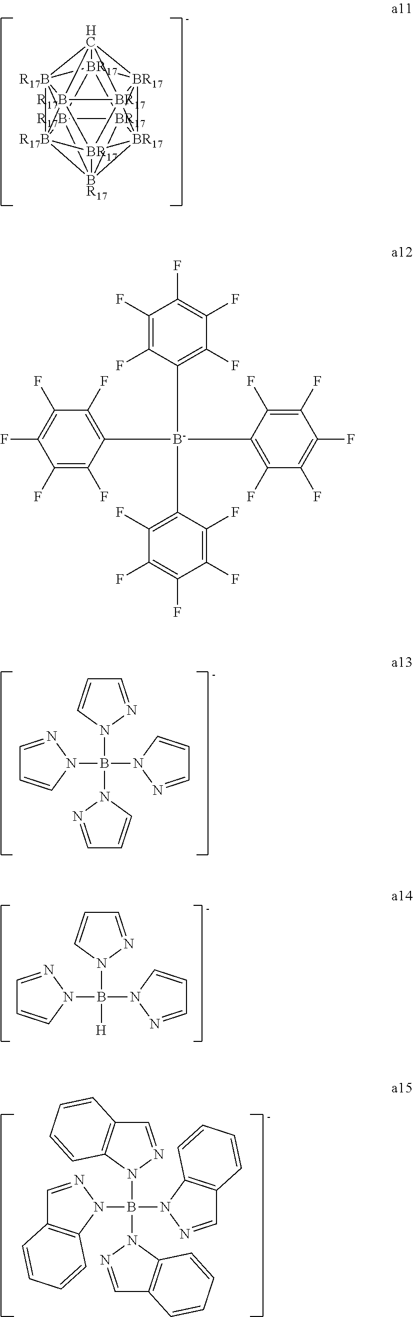

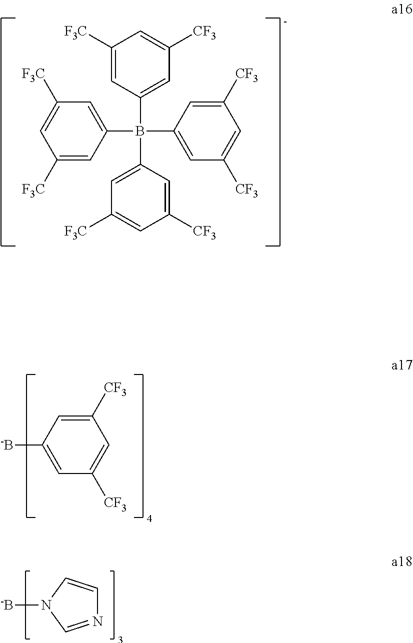

wherein R₇ is selected from hydrogen, deuterium, preferably substituted, preferably C₁-C₁₀ linear or branched alkyl, perfluorinated alkyl, partially fluorinated alkyl, or, preferably substituted, aryl, perfluorinated aryl, partially fluorinated aryl, or, preferably substituted, cycloalkyl, or, preferably substituted, alkenyl or, preferably substituted, alkynyl,
R₈ is selected from, preferably substituted, preferably C₁-C₁₀ linear or branched, alkanediyls, perfluorinated alkanediyls, partially fluorinated alkanediyls, or, preferably substituted, arylenes, perfluorinated arylenes, partially fluorinated arylenes, or, preferably substituted, cycloalkanediyls, or preferably substituted, alkenediyls or, preferably substituted, alkyndiyls,
R₉ to Ru are selected independently from a group including hydrogen, deuterium, preferably substituted, preferably C₁-C₁₀ linear or branched, alkyl, perfluorinated alkyl, partially fluorinated alkyl, preferably substituted aryl, perfluorinated aryl, partially fluorinated aryl, preferably substituted, cycloalkyl, preferably substituted, alkenyl and, preferably substituted, alkynyl,
R₁₂-R₁₃ are selected independently from, preferably substituted, perfluorinated C₁-C₂₀ alkyl, preferably substituted, C₁-C₂₀ alkyl, preferably substituted, perfluorinated aryls or, preferably substituted, aryls,
R₁₄ to R₁₆ are selected independently in each occurrence from a group including F, CN, preferably substituted, perfluorinated aryl or preferably substituted, perfluorinated heteroaryl,
R₁₇ is selected independently in each occurrence from a group including hydrogen, deuterium, halogen, methyl group, trifluoromethyl-group, or wherein the negatively charged anion is selected from [MCl₄]— with M=Al, Ga, [MF₆]— with M=As, Sb, Ir, Pt, [Sb₂F₁₁]—, [Sb₃F₁₆]—, [Sb₄F₂₁]—, CF₃SO₃, [B(CF₃)₄]—, [M(C₆F₅)₄] with M=B, Ga, [B(ArcF₃)₄]—, [HO(B(C₆F₅)₃)₂]—, [CHB₁₁H₅Cl₆]—, [CHB₁₁H₅Br₆]—, [CHB₁₁Me₅Br₆]—, [CHB₁₁F₁₁]—, [CEtB₁₁F₁₁]—, [B₁₂Cl₁₁NMe₃]—, [Al(OR^{PF})₄]—, [F(Al(OR^{PF})₃)₂]—, triflate, perchlorate, tetrafluoroborate, tetraphenylborate, or hexafluoroanions.

Example 12 is a mixture including a second electrically neutral organic compound, and the coordination compound according to any one of examples 1 to 11, wherein the coordination compound is imbedded into the at least one second electrically neutral organic compound, wherein the second organic compound has a triplet energy higher than 2.5 eV and/or wherein the coordination compound has a higher hole affinity compared to the second organic compound.

Example 13 is a compound including the coordination compound according to any one of examples 1 to 11, and polymer with a molecular weight Mn above 1000 g/mol, wherein the coordination is covalently attached to the polymer backbone.

In Example 14, the subject matter of Example 13 can in various embodiments include that the polymer molecule is an auxiliary organic molecule.

Example 15 is a contrast enhancement medium for magnet resonance tomography (MRT), the contrast enhancement medium including the coordination compound according to any one of examples 1 to 11, the mixture of example 12 or the compound of example 13.

Example 16 is an organic electronic device, including: a first electrode; a second electrode; and an organic layer arranged such that it is electrically interposed between the first and second electrodes, wherein the organic layer includes the coordination compound according to any one of examples 1 to 11, the mixture of example 12 or the compound of example 13 or 14.

In Example 17, the subject matter of Example 16 can in various embodiments include that the organic electronic device is an optoelectronic device, the optoelectronic device being at least one of an organic light emitting diode, an organic photodetector, or a photovoltaic cell.

In Example 18, the subject matter of any one of Examples 16 or 17 can in various embodiments include that the optoelectronic device further includes a wavelength conversion layer arranged in the light path of the organic layer.

Example 19 is a method of forming an organic device, the method including: forming a layer of the coordination compound according to any one of examples 1 to 11, of the mixture of example 12 or the compound of example 13 or 14, wherein the layer is deposited from a gas phase, in particular using an evaporation and/or sublimation process, and/or by a solution-based process.

In Example 20, the subject matter of Example 19 can in various embodiments include that forming the layer includes forming a first layer including the organic ligand and forming a second layer directly in contact with the first layer, wherein the second layer includes a divalent lanthanide salt.

In Example 21, the subject matter of Example 20 can in various embodiments include that the second layer is deposited by a solution-based process.

In Example 22, the subject matter of any one of Examples 20 or 21 can in various embodiments include that the layer is formed on or above a surface of a substrate, wherein the coordination compound includes a paramagnetic moment, and wherein the paramagnetic moment of the coordination compound is aligned perpendicular to the surface of the substrate.

In Example 23, the subject matter of any one of Examples 20 to 22 can in various embodiments include that the paramagnetic moment of the coordination compound is aligned during the deposition of the layer using an external electromagnetic field.

In Example 24, the subject matter of any one of Examples 20 to 23 can in various embodiments include that the paramagnetic moment of the coordination compound is aligned after the deposition of the layer using an external electromagnetic field.

Example 25 is a method to synthesize the coordination compound according to any one of examples 1 to 11, wherein a divalent lanthanide salt and an organic ligand according to formula (1) or formula (2) are reacted at pressure greater than or equal to about 1 bar, e.g. greater than 2 bar.

In Example 26, the subject matter of Example 25 can in various embodiments include that the divalent lanthanide salt is coordinated with an organic precursor and subsequently at least one of the groups R₁, R₂, R₃, R₄, R₅, R₆ is attached to the organic precursor.

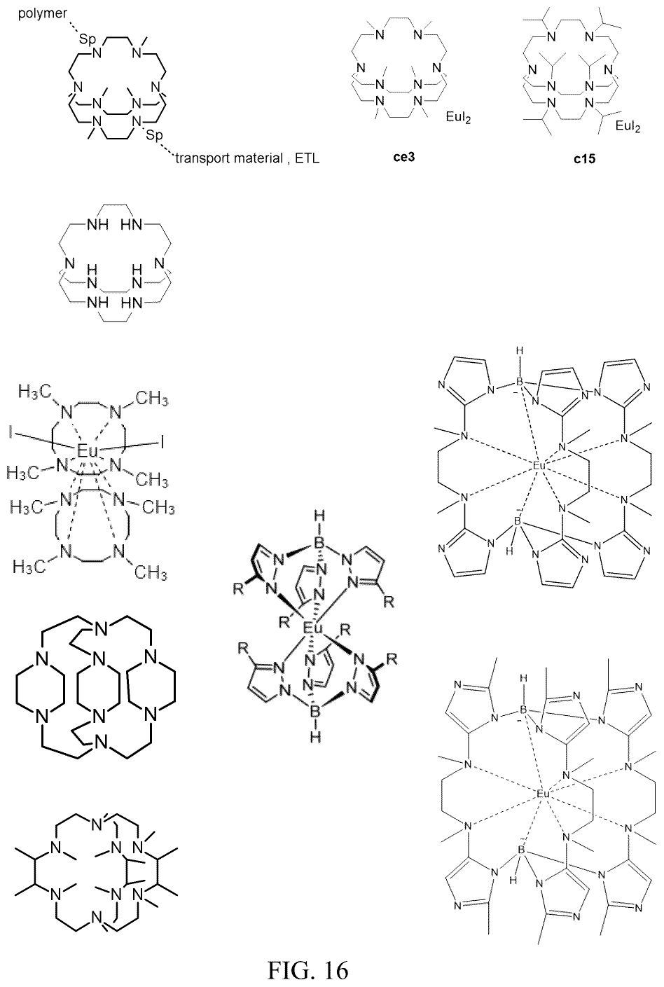

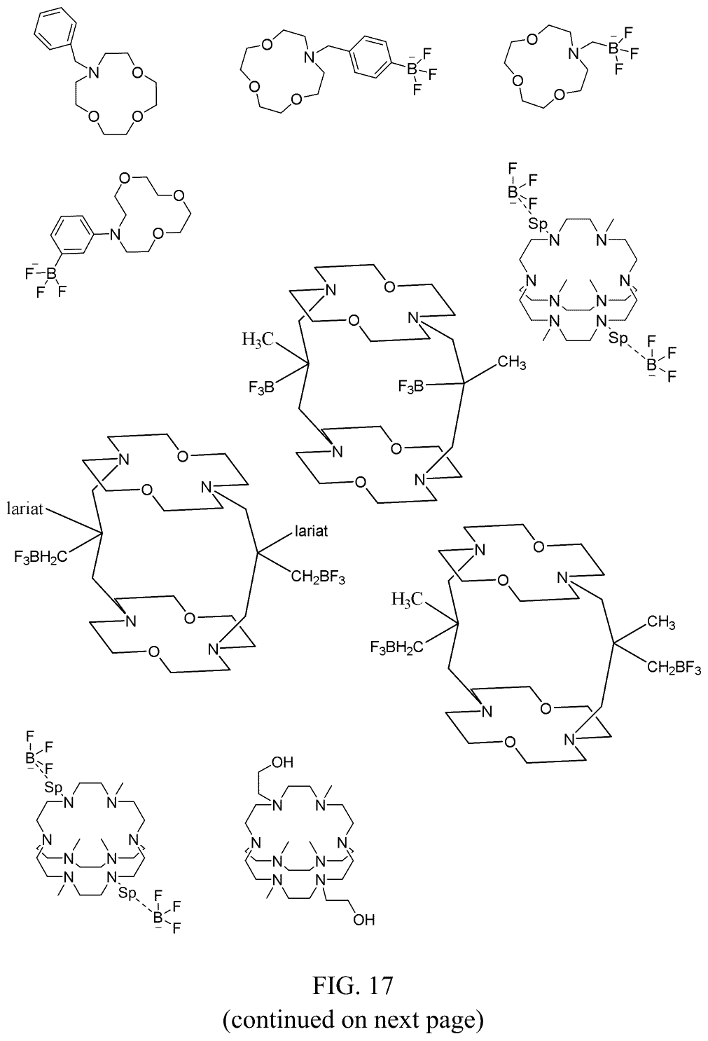

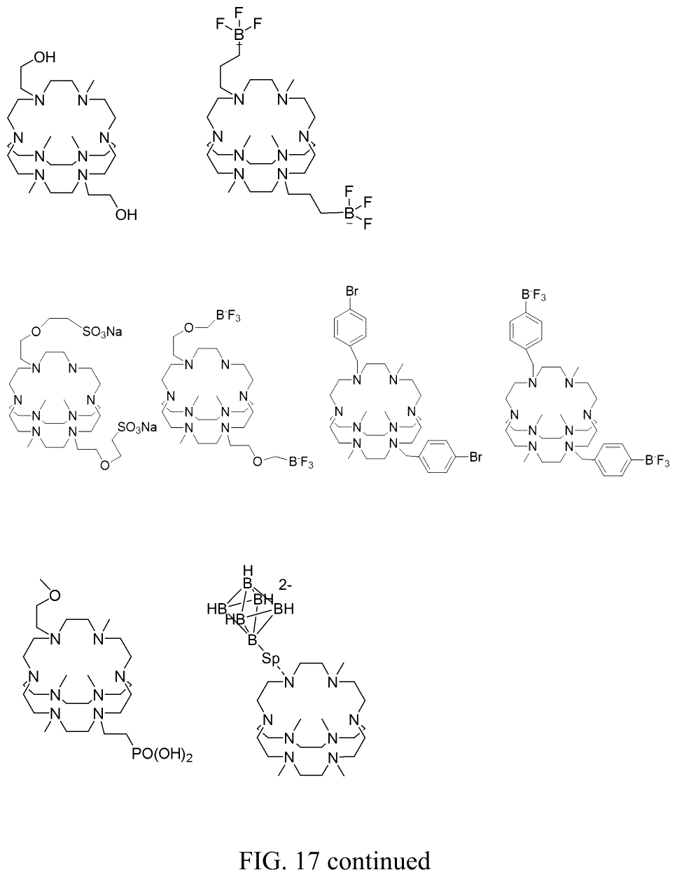

A FIG. 16 shows preferred examples of ligands and complexes; FIG. 17 shows preferred examples of ligands. The ligands are electrically neutral or negatively charged, as indicated.

Any of the above-described examples may be combined with any other example (or combination of examples), unless explicitly stated otherwise. The foregoing description of one or more implementations provides illustration and description, but is not intended to be exhaustive or to limit the scope of aspects to the precise form disclosed. Modifications and variations are possible in light of the above teachings or may be acquired from practice of various aspects. Any of the above-described examples may be combined with any other example (or combination of examples), unless explicitly stated otherwise. The foregoing description of one or more implementations provides illustration and description, but is not intended to be exhaustive or to limit the scope of aspects to the precise form disclosed. Modifications and variations are possible in light of the above teachings or may be acquired from practice of various aspects.

While the invention has been particularly shown and described with reference to specific aspects, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

### Experimental

### Synthesis

The following procedures are, unless indicated otherwise, carried out under a protective gas atmosphere in an oven dried glassware. The solvents and starting reagents can be purchased from any commercial source, such as Merck or Acros. Ligands, used for the synthesis of the coordination compound c9, c11, c15 to c30 (see also FIG. 6A and FIG. 11) were synthesized from according to the reaction scheme:

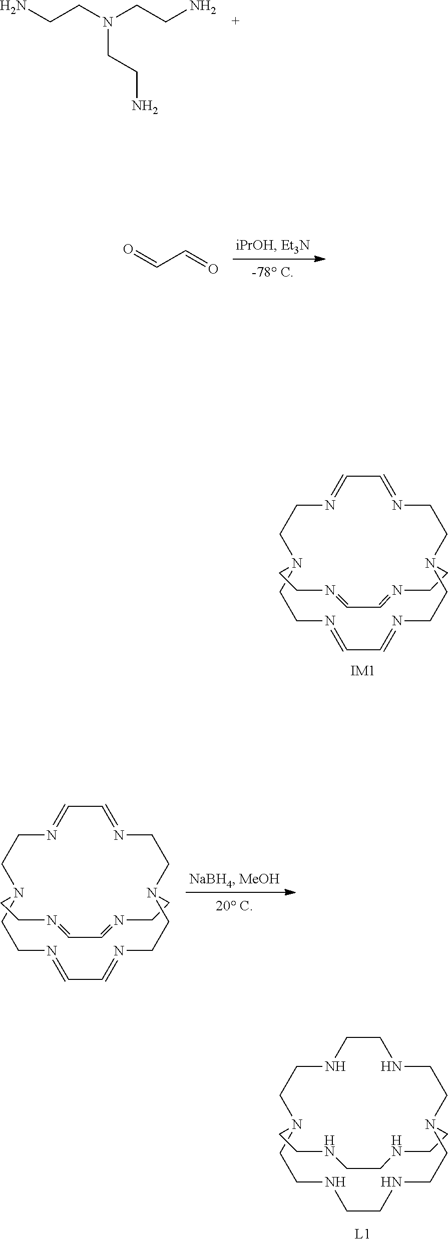

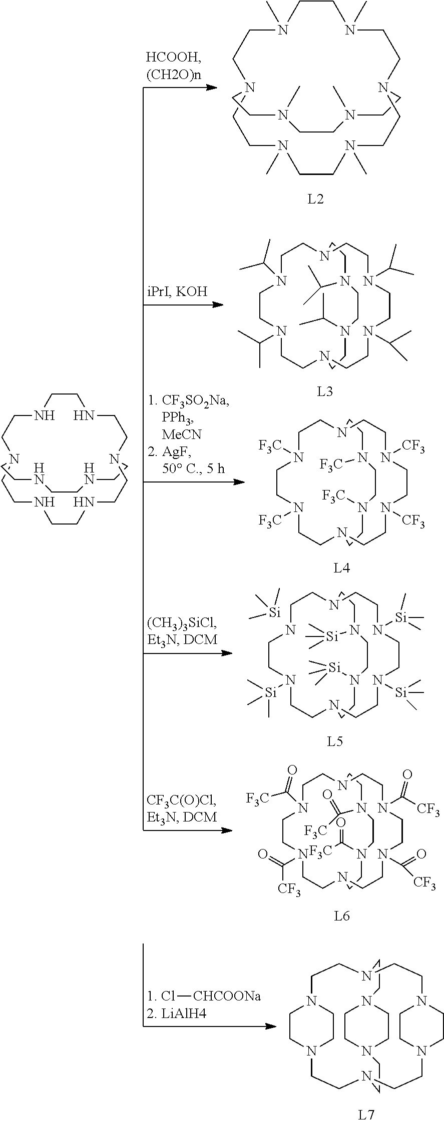

### Synthesis of IM1

The synthesis was done using modified procedure of Redko as mentioned above. Tren (9.73 g, 66.6 mmol), Et3N (25 ml) and i-PrOH PrOH (500 ml) were placed in a 1 L three-neck round-bottom flask equipped with an addition funnel and mechanical stirrer. The resulting solution was cooled to −78° C. (dry ice —i-PrOH bath), and a solution of aq. glyoxal (40%, 14.5 g, 0.1 mol) in i-PrOH (250 ml) was then added at a rate of 2 drops/sec with vigorous stirring. Subsequently, the reaction mixture was allowed to warm to room temperature. Next, the solvent was removed on a rotary evaporator yielding a brown solid, which was extracted 5 times with toluene (1500 ml in total). Combined extracts were concentrated on rotary evaporator to the volume of about 50 ml, precipitated white solid was separated by suction filtration, washed with cold toluene (50 ml) and hexane (50 ml), dried in vacuum yielding 7.77 g(65%) of white solid.

1H NMR (CDCl3): δ=2.7 (br. 12H), 3.51 (br. 12H), 7.7 (s, 6H)

MS: m/z=358(M⁺)

### Synthesis of L1

2 L round-bottom flask was charged with 5 g (14 mmol) of IM1 and 120 ml methanol. To the resulting solution, 10 g (0.26 mol) of NaBH was added within 3 h in 5 portions. After the addition was complete, the reaction solution was stirred at 25° C. overnight. 100 ml of deionized water was added to the reaction mixture with stirring, the solvents were removed under reduced pressure yielding a colorless oil. Residue was stirred with 50 ml of deionized water, the white solid was separated by filtration and dried in vacuum at 60° C. Yield: 4.8 g (92%)

H-NMR (CDCl₃): δ=2.50 (t, 12H,); 2.74 (t, 12H); 2.77 (s, 12H).

MS: m/z=371 (M⁺).

### Synthesis of Compound L2

To a solution of L1 (2.50 g, 6.7 mmol) in 90% formic acid (100 ml), a large excess of paraformaldehyde (12.5 g) was added. The mixture was heated under vigorous stirring. After complete dissolution, the solution was refluxed for 3 days under Argon and then evaporated. H20 (200 ml) was added to the residue, the solution was basified to pH 10 by addition of KOH and extracted with CHCl3 (3×150 mL). The extract was evaporated and the obtained yellow oil dissolved with an aq. HCl. The solution was washed with toluene, then basified to pH10 by addition of KOH and extracted with hexane (3×100 ml). The combined extract was washed with brine (20 ml), dried over anhydrous Na₂SO₄, filtered and evaporated to the dryness yielding yellowish solid (1.56 g, 51%)

H-NMR (CDCl₃): δ=2.29 (s, 18H,); 2.56 (m, 24H); 2.72 (s, 12H).

MS: m/z=455 (M⁺).

### Synthesis of Compound L3

1 L three-neck round-bottom flask, equipped with magnetic stirring bar, Soxhlet extractor loaded with 10 g of anhydrous MgSO4, and reflux condenser was charged with anhydrous toluene (500 ml), IM2 (2.50 g, 6.7 mmol) and isopropyl iodide (6 eq. 40.5 mmol, 6.88 g) and the mixture was heated under reflux for 2 h. A solid potassium hydroxide (6 eq., 40.5 mmol, 2.27 g) was added and the resulting mixture was heated under reflux for 20 h. The mixture was filtered and extracted 5 times with 2M solution of HCl (100 ml). The combined extracts were washed with toluene (50 ml), then basified to pH10 by addition of KOH and extracted with hexane (3×100 ml). The combined extract was washed with brine (20 ml), dried over anhydrous Na₂SO₄, filtered and evaporated to the dryness yielding crude product, which was further purified by column chromatography. Yield: 2.5 g (59%)

H-NMR (CDCl₃): δ=0.92 (d, 36H); 2.52 (m, 36H); 2.79 (m, 6H)MS: m/z=623 (M⁺).

### Synthesis of Compound L4

In a nitrogen-filled glovebox, a 10 mL oven-dried reaction vessel was charged with CF3SO2Na (7.5 mmol, 1.17 g, 1.5 equiv.) and PPh3 (15 mmol, 3.93 g, 3 equiv.), then a solution of IM2 (1.8 g, 5 mmol)

in MeCN (25 mL) was added. The resulting solution was stirred at room temperature for 1 h. After that, AgF (2.25 mmol, 286 mg, 4.5 equiv) was added. The resulting mixture was further stirred at 50° C. for 5 h. After cooling to room temperature, the volatiles were removed under vacuum and the residue was purified by column chromatography to give the title compound. Yield: 2.51 g (83%)

H-NMR (CDCl₃): δ=2.29 (s, 18H,); 2.56 (m, 24H); 2.72 (s, 12H)

19F-NMR: (CDCl3) δ=−68.10

### Synthesis of Compound L5

A clean and dry 250 ml 3-necked round bottom flask, equipped with a magnetic stir bar, reflux condenser fitted with a nitrogen inlet and thermometer may be charged with L1 (7.41, 20 mmol). The flask may be sealed with a septum, an atmosphere may be replaced by nitrogen. Anhydrous trimethylamine (132 mmol, 6.6 eq, 13.3 g) may be added through a septum with a syringe, followed by an addition of anhydrous dichloromethane (100 ml) via double tipped cannula. The septum may be replaced by an addition funnel, which was charged with a solution of chlorotrimethylsilane (CTMS) (132 mmol, 6.6 eq., 14.3 g), dissolved in 50 ml of anhydrous dichloromethane. The reaction mixture may be cooled down to −10° C. and the solution of CTMS was added dropwise with a rate, which allows to keep the temperature of the reaction mixture below 0° C. After the addition may be finished, the cooling bath may be removed and the mixture may be stirred at room temperature (3 h) and under reflux (6 h) in order to complete the reaction. The mixture was cooled using ice bath and quenched by addition of water. Organic layer may be separated, washed with water (3×20 ml), brine (1×20 ml), dried over sodium sulfate and evaporated to dryness yielding a crude product, which was purified by column chromatography (SiO₂, ethylacetate:methanol 50:1). Yield: 11.7 (73%), white powder.

H-NMR (CDCl₃): δ=0.08 (s, 54H); 2.47 (m, 12H); 2.65 (m, 24H)

### Synthesis of Compound L6

A clean and dry 100 ml 3-necked round bottom flask, equipped with a magnetic stir bar, reflux condenser fitted with a nitrogen inlet and thermometer may be charged with L1 (3.7 g, 10 mmol). The flask may be sealed with a septum, an atmosphere may be replaced by nitrogen. Anhydrous trimethylamine (70 mmol, 7 eq, 7.08 g) may be added through a septum with a syringe, followed by an addition of anhydrous dichloromethane (50 ml) via double tipped cannula. The septum may be replaced by an addition funnel, which was charged with a solution of trifluoroacetic anhydride (66 mmol, 6.6 eq., 13.88 g), dissolved in 25 ml of anhydrous dichloromethane. The reaction mixture may be cooled down to −10° C. and the solution of anhydride was added dropwise with a rate, which allows to keep the temperature of the reaction mixture below 0° C. After the addition may be finished, the cooling bath may be removed and the mixture may be stirred at room temperature (3 h) in order to complete the reaction. The mixture was cooled using ice bath and quenched by addition of water. Organic layer may be separated, washed with water (3×20 ml), brine (1×20 ml), dried over sodium sulfate and evaporated to dryness yielding a crude product, which was purified by column chromatography (SiO₂, ethylacetate:methanol 50:1). Yield: 5.5 g (58%), off-white powder.

H-NMR (CDCl₃): δ=2.62 (m, 12H); 3.3 (m, 24H)

19F-NMR: (CDCl₃) δ=−76.30

### Synthesis of Compound L7

A mixture of 3, 7 g of L1 (10 mmol) and 4.66 g of ClCH2COONa (40 mmol, 33% excess) was refluxed in 50 mL of 1-butanol in a 100 mL round-bottom flask for 12 h in a nitrogen atmosphere. The butanol was then evaporated in a N2 stream, the resulting mixture was redissolved in 50 mL of 2-propanol and filtered, and the 2-propanol was evaporated to yield a crude oily mixture of polyam ides. After addition of 2.0 g of LiAIH₄ and 50 mL of fresh THF to the mixture, it was refluxed for 12 h in a nitrogen atmosphere. After the mixture was cooled, excess LiAIH₄ was quenched by dropwise addition of 10 mL of saturated aqueous NaOH and 20 mL of 2-propanol. Precipitated aluminium salts were separated by filtration, filtrate evaporated to a dryness and a crude product was purified by column chromatography (SiO₂, ethylacetate:methanol 10:1 to 1:1). Yield 2.8 g (53%), pale yellow oil, which crystalizes upon storage.

H-NMR (CDCl₃): δ=2.43 (b, 10H), 2.72 (b, 38H)

ESI-MS:449 [M⁺H]⁺, 471[M⁺Na]⁺

### General Procedures for the Synthesis of the Coordination Compounds

Method 1: A solution of corresponding metal iodide (1 eq.) in THF was added drop wise to the solution of corresponding ligand in THF. Precipitated metal complex was separated by suction filtration, washed with THF and dried in vacuum yielding pure product.

Method 2: In a dry box, an autoclave was charged with metal iodide (0.5 mmol), Ligand (0.5 mmol) and anhydrous DMF. The autoclave was sealed and heated at 170° C. for 12 h. Subsequently, solvent was evaporated to dryness and the residue was re-crystallized from an appropriate solvent yielding a pure coordination compound.

Method 3: To the solution of corresponding iodide stabilized coordination compound in methanol, an excess of an aqueous solution of ammonium hexafluorophosphate or Na[BArF₂₄] was added with stirring. Methanol was removed by vacuum destilation, the precipitate was separated by suction filtration, washed with deionized water and dried. The crude product was purified by re-crystallization in an appropriate solvent or by sublimation.

Method 4: This method was applied as an alternative to method 2 for the synthesis of a sterically hindered complexes according to the scheme:

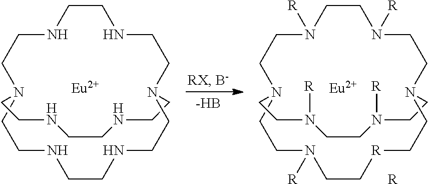

As a starting compound, complex c10 have been used in most cases. To the suspension of c10 (1 eq.) in tetrahydrofurane (25 ml), triethylamine (6.6 eq) is added followed by a dropwise addition of a solution of the corresponding electrophiles (6 eq.) in tetrahydrofurane at the temperature below 0° C. The reaction mixture is stirred at RT or under reflux for 24 h, the product is separated by suction filtration, washed with tetrahydrofurane and dried in vacuum at 40° C. Final purification of the crude product was achieved by re-crystallization in an appropriate solvent or by sublimation.
The table below summarizes synthesis yields and elemental analysis data for various coordination compounds that have been synthesized according to different methods 1 to 4:

| Starting | | | Yield | Elemental Analysis, |
|---|---|---|---|---|
| material | Method | Product | (%) | calc./found |
| L1 | 1 | C9 | 89 | C, 36.43; H, 7.13; N, 18.88/C, 36.6; H, 7.17; N, 18.84 |
| L2 | 1 | c11 | 99 | C, 27.64; H, 4.64; N, 3.58/C, 26.9; H, 4.71; N, 3.57; |
| L3 | 2 | c15 | 58 | C, 24.34; H, 3.06; N, 9.46/C, 24.62; H, 3.08; N, 9.61 |
| L3 | 2 | c16 | 42 | C, 41.18; H, 7.49; N, 10.67/C, 42.04; H, 7.6; N, 9.47 |
| L4 | 1 | c21 | 81 | C, 24.34; H, 3.06; N, 9.46/C, 25.02; H, 3.17; N, 9.60 |
| L4 | 1 | c22 | 75 | C, 23.91; H, 3.01; N, 9.30/C, 24.03; H, 3.03; N, 9.31 |
| L5 | 2 | c27 | 33 | C, 35.75; H, 7.50; N, 9.26/C, 36.05; H, 7.70; N, 9.15 |
| c10 | 4 | c27 | 84 | C, 35.75; H, 7.50; N, 9.26/C, 36.05; H, 7.70; N, 9.33 |
| L5 | 2 | c28 | 37 | — |
| L6 | 2 | c33 | 51 | C, 34.33; H, 5.19; N, 10.68/C, 34.92; H, 5.17; N, |
| | | | | 10.81 |
| c10 | 4 | c33 | 89 | C, 34.33; H, 5.19; N, 10.68/C, 34.92; H, 5.23; N, |
| | | | | 11.07 |
| L6 | 2 | c34 | 60 | |
| c15 | 3 | c17 | 88 | C, 40.60; H, 7.38 N, 10.52;/C, 41.05; H, 7.4 N, 10.4; |
| c15 | 3 | c19 | 97 | — |
| c16 | 3 | c18 | 91 | C, 39.81; H, 7.24; N, 10.32/C, 40.2; H, 7.31; N, 10.1 |
| c16 | 3 | c20 | 98 | C, 47.61; H, 4.08; N, 4.44/C, 48.02; H, 4.13; N, 4.01 |
| c21 | 3 | c23 | 84 | C, 23.62; H, 2.97; N, 9.18/C, 23.64; H, 2.95; N, 9.15 |
| c21 | 3 | c25 | 92 | C, 39.78; H, 2.28; N, 4.22/C, 39.54; H, 2.13; N, 4.08 |
| c22 | 3 | c24 | 75 | C, 23.22; H, 2.92; N, 9.03/C, 23.20; H, 2.86; N, 9.13 |
| c22 | 3 | c26 | 79 | — |
| c27 | 3 | c29 | 87 | |
| c27 | 3 | c31 | 91 | — |
| c28 | 3 | c30 | 83 | |
| c28 | 3 | c32 | 97 | — |
| c33 | 3 | c35 | 88 | C, 25.95; H, 2.61; N, 8.07/C, 25.90; H, 2.63; N, 8.06 |
| c33 | 3 | c37 | 95 | — |
| c34 | 3 | c36 | 95 | C, 25.56; H, 2.57; N, 7.95/C, 24.97; H, 2.7; N, 7.69 |
| c34 | 3 | c38 | 98 | — |
| L7 | 1 | c39 | 97 | C, 33.74; H, 5.66; N, 13.1/C, 33.92; H, 5.75; N, 13.03 |
| L7 | 1 | c40 | 79 | C, 32.92; H, 5.53; N, 12.8/C, 33.1; H, 5.6; N, 12.74 |
| C39 | 3 | c41 | 85 | C, 32.37; H, 5.43; N, 12.58/C, 32.45; H, 5.51; N, |
| | | | | 12.21 |
| C39 | 3 | c43 | 67 | |
| C40 | 3 | c42 | 88 | C, 44.15; H, 2.47; N, 5.72/C, 44.5; H, 2.6; N, 5.91 |
| C40 | 3 | | 75 | |

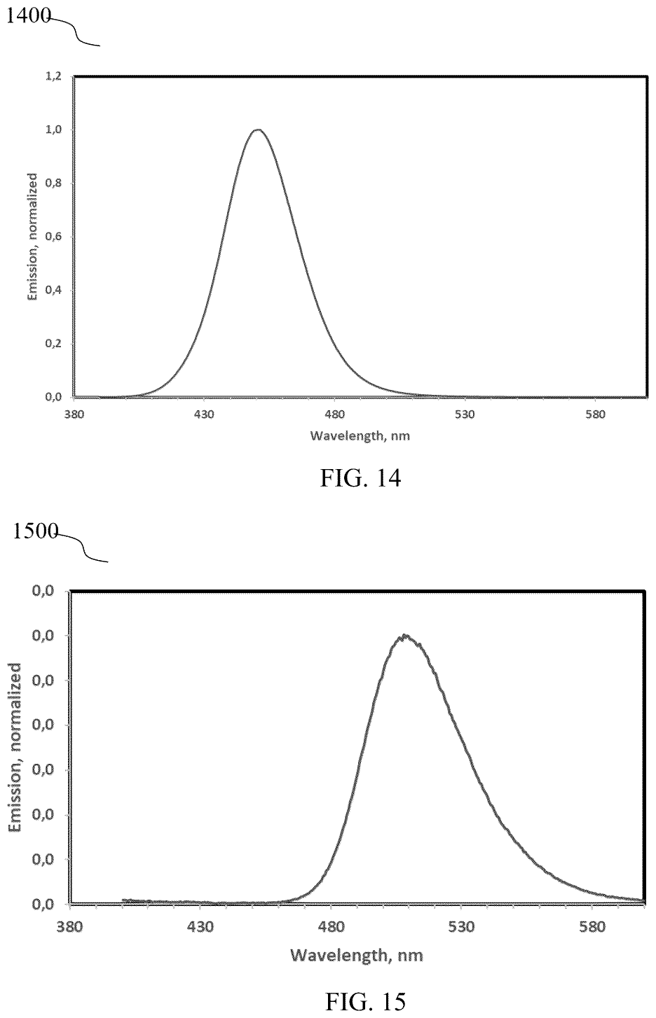

FIG. 14 illustrates an emission spectrum 1400 of a coordination compound according to Ce15 (FIG. 6A) measured in methanol after excitation at 390 nm that may be used in the organic layer 106 according to various embodiments.

FIG. 15 illustrates an emission spectrum 1500 of a coordination compound according to C₂₇ (FIG. 6A) measured in solid state after excitation at 350 nm that may be used in the organic layer 106 according to various embodiments. The material was synthesized using Method 4 from the start compound C₁₀. The spectrum was taken from a solid state sample after excitation at 350 nm.

### Experimental Part

### Devices

Representative embodiments of an organic electronic device according to various embodiments will now be described, including a detailed description of the fabrication process of the organic electronic device. Yet, it may be understood that neither the specific techniques for fabrication of the device, nor the specific device layout, nor the specific compounds are intended to limit the scope.

Material Definitions:

| | |
|---|---|
| ITO: | Indium tin oxide transparent anode |
| PEDOT:PSS: | poly(3,4-ethylenedioxythiophene) polystyrene sulfonate. |
| | Used was EL4083 from Heraeus. |
| PVK: | Poly(9-vinylcarbazole). The material was purchased |
| | from Sigma-Aldrich with average molecular weight |
| | of ~1.100.000 g/mol. |
| DPEPO: | (Oxybis(2,1-phenylene))bis(diphenylphosphine oxide). |
| | The material was purchased from Osilla. |
| TPBI: | 1,3,5-tris(1-phenyl-1H-benzo[d]imidazol-2-yl)benzene. |
| | The material was purchased from Osilla. |
| LIF: | Lithiumfluorid |
| Al: | Aluminum |

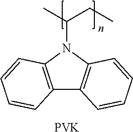

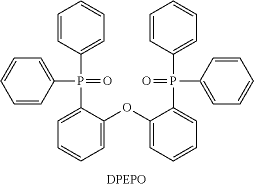 [SMILES: O=P(c1ccccc1)(c1ccccc1)c1ccccc1Oc1ccccc1P(=O)(c1ccccc1)c1ccccc1]

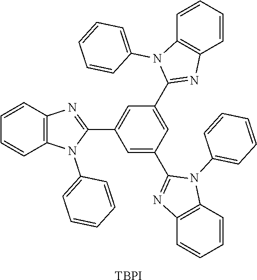

Two coordination compounds ce3, c15 illustrated in FIG. 16 may be tested in two different organic electronic device stacks. Here, ce3 and c15 may be according to various embodiments. The synthesis of ce3 and C₁₅ is described above, the synthesis of the comparative examples follows similar routes starting from commercially available ligands.

### Example Series 1

Four devices were prepared with the generic organic electronic device layer sequence: ITO (100 nm)/PEDOT:PSS (30 nm)/PVK (10 nm)/coordination compound (4 nm)/DPEPO (5 nm)/TPBI (35 nm)/LIF (0.5 nm)/AL (100 nm). The organic light emitting devices were fabricated on glass substrates, pre-coated with transparent ITO anode. The substrates were thoroughly cleaned using various solvents in ultrasonic bath and exposed to UV ozone plasma for 10 min. Next, PEDOT:PSS was spin-coated from filtered solution at 4000 rpm giving about 30 nm thick film. The coated substrates were annealed for 15 min at 135° C. in air to remove residual solvents. Subsequently, the substrates were loaded into a nitrogen containing glovebox, where an electron blocking layer with high triplet energy, namely the hole transporting polymer PVK was spincoated from a 5:95, chlorobenzene:chloroform, 3 mg/ml, degassed solution at 6000 rpm resulting in approximately 10 nm film thickness.

All solvents used in the glovebox were beforehand thoroughly degassed by using three freeze-thaw cycles to remove any oxygen. The coordination compounds according to various embodiments ce3 and c15 were dissolved in methanol at 0.5 mg/ml. These solutions were used to cast approximately 4 nm thick, pure coordination compound layers on to the PVK layer by spin coating at 6000 rpm. By using methanol, the coordination compound solution does not re-dissolve the PVK underlying layer. Next, the substrates were loaded directly (without exposure to ambient atmosphere) from the glovebox into a thermal evaporation system operating at a base pressure of ˜1×10−6 Torr. Here, the organic electronic device fabrication continued by first evaporating a 5 nm thick layer of the high triplet energy, hole blocking material DPEPO, followed by a 35 nm thick layer of the electron transport material TPBI. Next a 0.9 nm thick electron injection layer of LiF was thermally evaporated, before finishing the device by thermally evaporating a 100 nm thick aluminum cathode layer. The cathode layer was evaporated through a shadow mask, which, together with the pre-structured ITO anode, defines the active emission area of the organic electronic device to 2×2 mm². Subsequently, the substrates were again loaded into the glovebox without exposure to ambient atmosphere, where a glass substrate was glued just above the active device areas in order to protect them from oxidation under ambient may be condition. The device characteristics were measured using a calibrated integrating sphere fiber coupled to a CCD camera.

All devices had their current turn-on (current density reaching 0.01 mA/cm²) at about 4 V. At about 7 V the current density reached ˜10 mA/cm², which may represent typical driving conditions for organic electronic device flat panel displays. The luminance at 7 V applied voltage may be summarized in Table 2.

TABLE 2

| organic electronic | |
|---|---|
| device including | Luminance cd/m² |
| Compound ce3 | 32 |
| Compound C15 | 114 |

Here, the system starts to detect signals at about 2 cd/m2 for deep blue emitting devices of 2×2 mm².

Surprisingly, all devices containing the compounds according to various embodiments, ce3 and C₁₅, as emitting material, exhibit intensive deep blue light emission upon application of sufficient voltage. Here, the observed emission spectra, after electrical excitation, may be in excellent agreement with emission characteristics measured in methanol, compare FIG. 14. In particular, after electrical excitation, deep blue, Gaussian-shaped, single line emission spectra peaking at ˜460 nm without any vibronic overtones may be observed. This may be clear evidence that the light emitted by the organic electronic devices according to various embodiments may be emitted from the coordination compounds as described by formula illustrated in FIG. 1A.

### Example Series 2

Four devices were prepared with the generic organic electronic device layer sequence: ITO (100 nm)/PEDOT:PSS (30 nm)/PVK (10 nm)/DPEPO: coordination compound (20 wt %)/(35 nm)/TPBI (35 nm)/LIF (0.5 nm)/AL (100 nm). The organic light emitting devices were fabricated identically to the ones of Series 1 described above, except for the emission layer and the hole blocking layer. For fabrication of the emission layer, first a master solution of DPEPO was prepared by dissolving it in degassed methanol. Next, the coordination compounds according to various embodiments ce3 and C₁₅ were dissolved in degassed methanol. The 2 solutions containing the coordination compounds were each mixed with the master solution containing the DPEPO such that the overall content of solute in the solution was 6 mg/ml of which 1 mg/mloriginates from the coordination compounds and 5 mg/ml originates from DPEPO. Those solutions were used to cast approximately 35 nm thick mixed layers of the coordination compounds with DPEPO onto the PVK layers by spin coating at 6000 rpm. The organic electronic devices were finished alike to Series 1 described above, however, without adding an extra DPEPO hole blocking layer.

All devices had their current turn-on (current density reaching 0.01 mA/cm²) at about 5 V. At about 8 V the current density reached ˜10 mA/cm², which may represent typical driving conditions for organic electronic device flat panel displays. The luminance at 8 V applied voltage may be summarized in Table 3.

TABLE 3

| organic electronic | |
|---|---|
| device including | Luminance cd/m² |
| Compound ce3 | 26 |
| Compound C15 | 221 |

Surprisingly, all devices that use the coordination compound as active emitting material, ce3 and C₁₅, mixed with DPEPO host, exhibit deep blue intensive light emission upon application of sufficient voltage. Here, the observed emission spectra after electrical excitation may be again in excellent agreement with emission characteristics measured in methanol, shown in FIG. 14. In particular after electrical excitation, deep blue, Gaussian-shaped, single line emission spectra peaking at ˜460 nm without any vibronic overtones may be observed. This may be clear evidence that the light emitted by the organic electronic devices according to various embodiments may be emitted from the coordination compounds as described by formula illustrated in FIG. 1A.

The following is a list of alternative embodiments in addition to the attached claims.
1 A metal-organic coordination compound, wherein the coordination compound comprises at least one divalent lanthanide coordinated by a cyclic organic ligand according to formula 1:

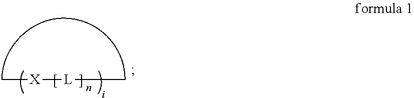

wherein
   i is larger than 3; and
   n is equal to 1, 2, or 3; and
   L for each occurrence is independently selected from arylenes or biradical fragments of

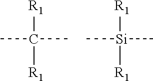

and
X is independently selected for each occurrence from the group of:

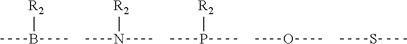

wherein R₁ and R₂ are any covalently bound substituents being identical or different in each occurrence of n and i; and
wherein R₁ and/or R₂ are at least in 3 occurrences not hydrogen.
2. The compound according to embodiment 1, wherein the divalent Lanthanide is Europium or Ytterbium.
3. The compound according to embodiment 1, wherein R₁ and R₂ are connected to each other thereby forming a polycyclic ligand, wherein at least two R₂ are connected forming a bridge to form the polycyclic ligand.
4. The coordination compound according to any one of embodiments 1 to 3, wherein the organic ligand according to formula (1) is electrically neutral.
5. The coordination compound according to any one of embodiments 1 to 4, wherein the coordination compound comprises at least one negatively charged anion, which is not covalently bound to the organic ligand.
6. The coordination compound according to embodiment 5, wherein the negatively charged anion comprises more than one atom.
7 The coordination compound according to any one of embodiments 2 to 6, the cyclic organic ligand of formula 1 having a structure according to formula 2:

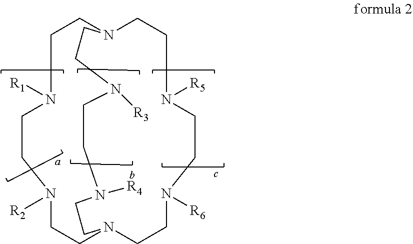

wherein
   R₁, R₂, R₃, R₄, R₅, R₆ independently in each occurrence represent an organyl group, comprising at least one carbon atom and at least one additional atom; whereby the additional atom is not a hydrogen or not an organoheteryl group, and
   a, b, c are each independently an integer of 0 or more.
8. The coordination compound according to embodiment 7, wherein a, b and c are each equal to 1.
9. The coordination compound according to embodiment 7 or 8, wherein R₁, R₂, R₃, R₄, R₅, R₆ are in each occurrence independently selected from the group of f1 to f78, wherein f1 to f78 are:

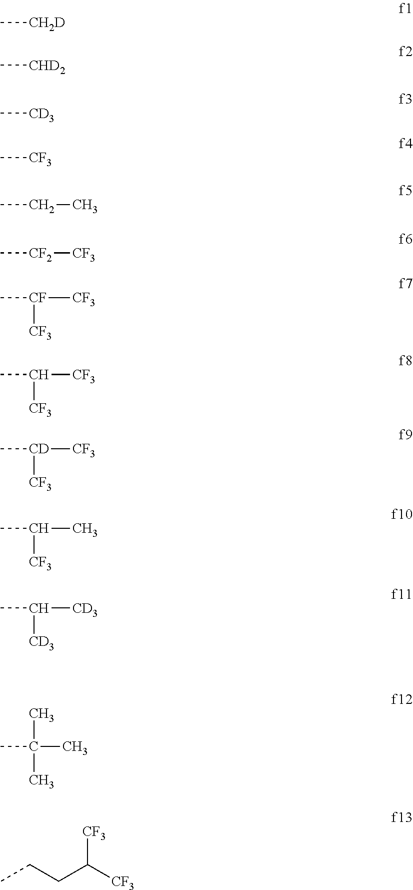

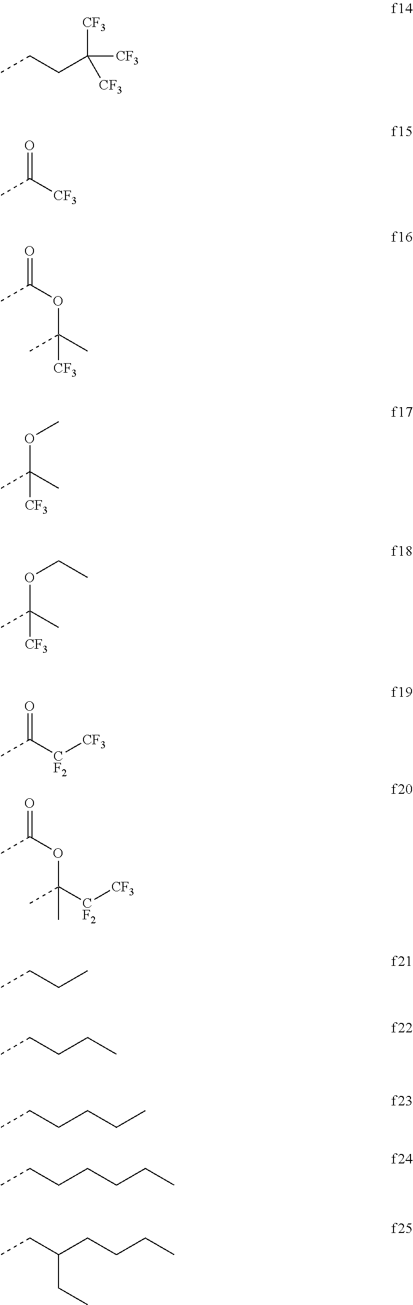

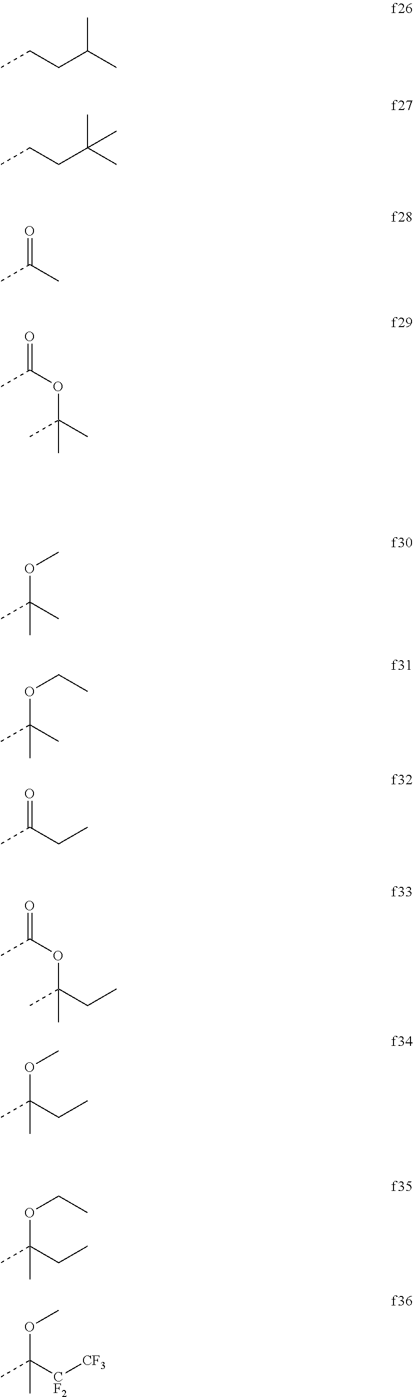

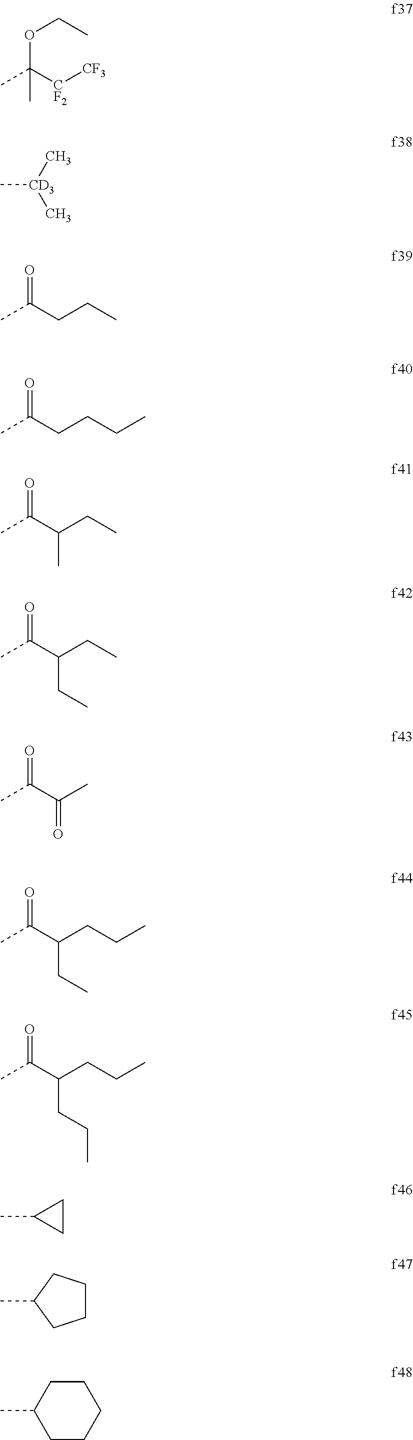

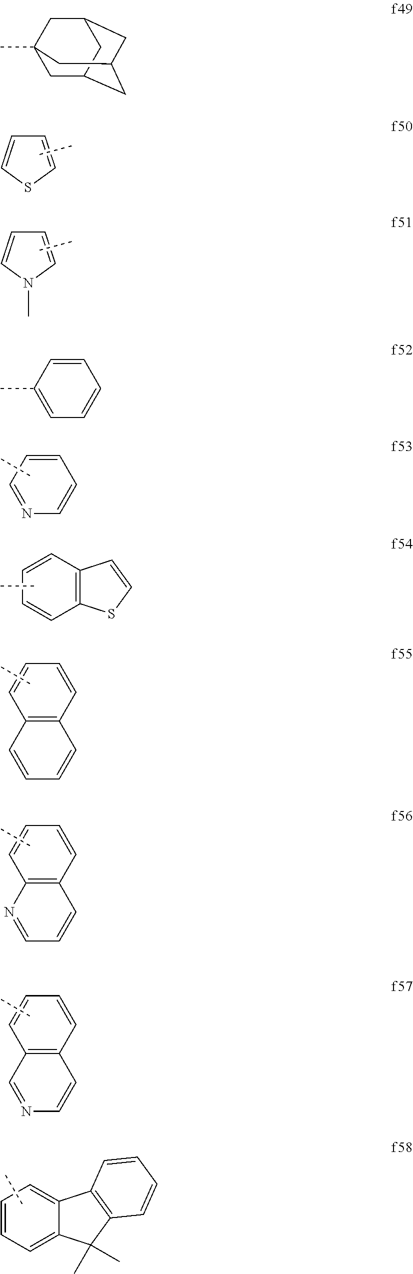

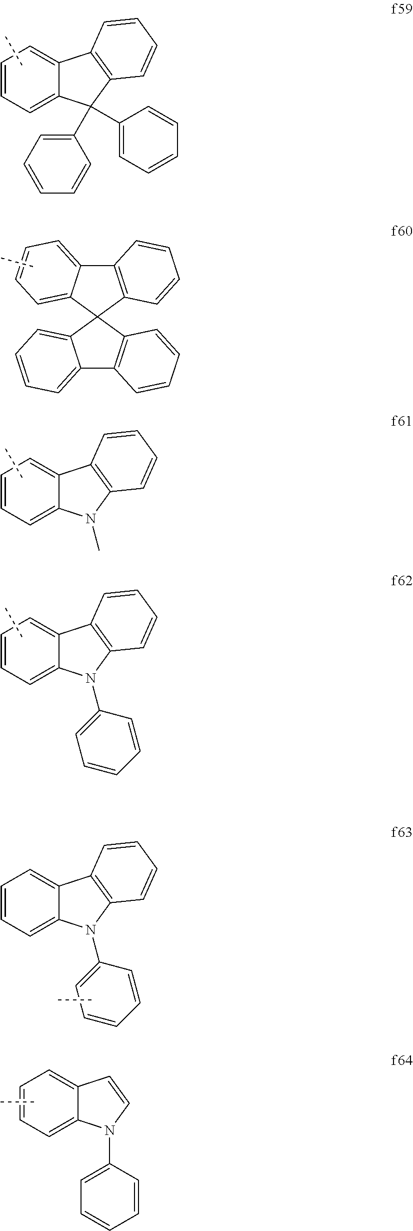

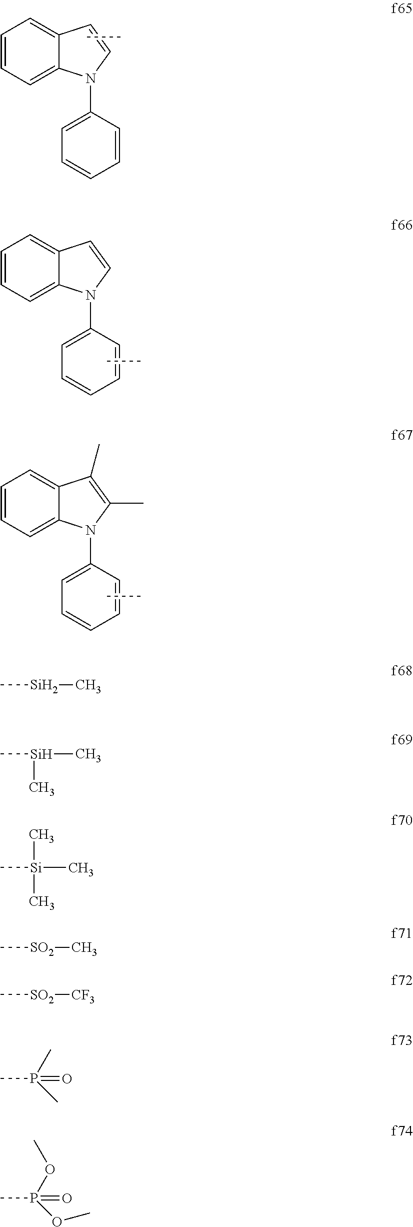

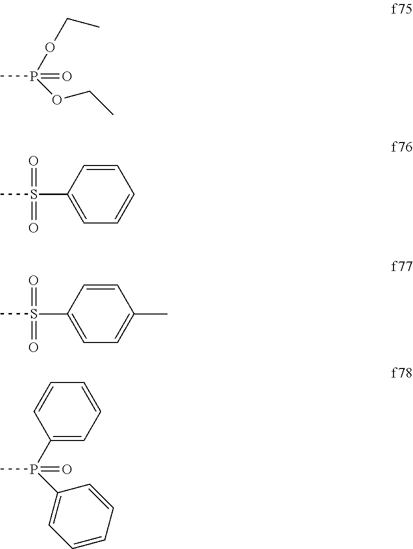

wherein a dash line represents the preferred connection point.
10. The coordination compound according to anyone of embodiments 7 to 9, wherein the coordination compound comprises at least one negatively charged anion, which is not covalently bound to the organic ligand, wherein the negatively charged anion is at least one selected from the group of a1 to a41, wherein a1 to a41 are:

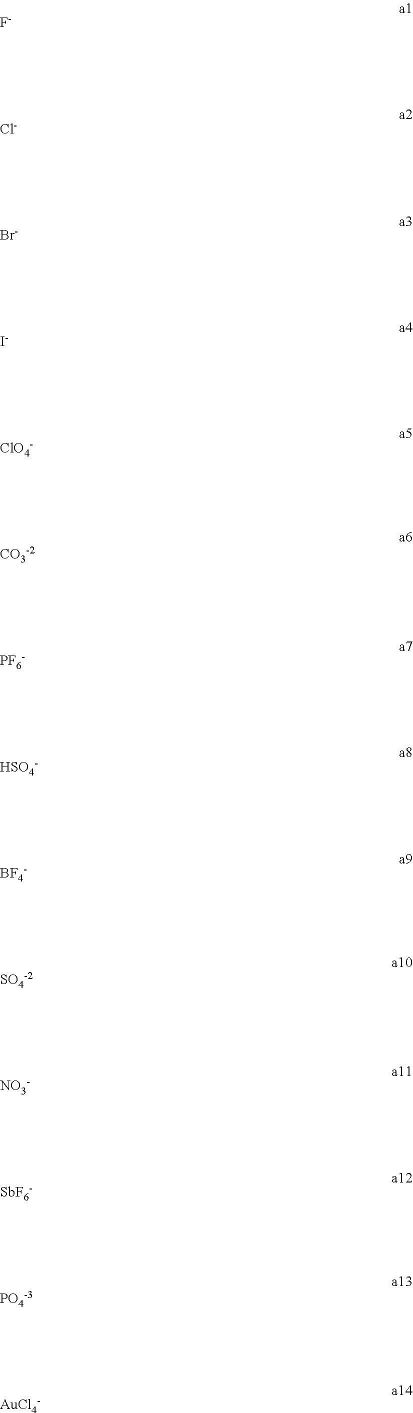

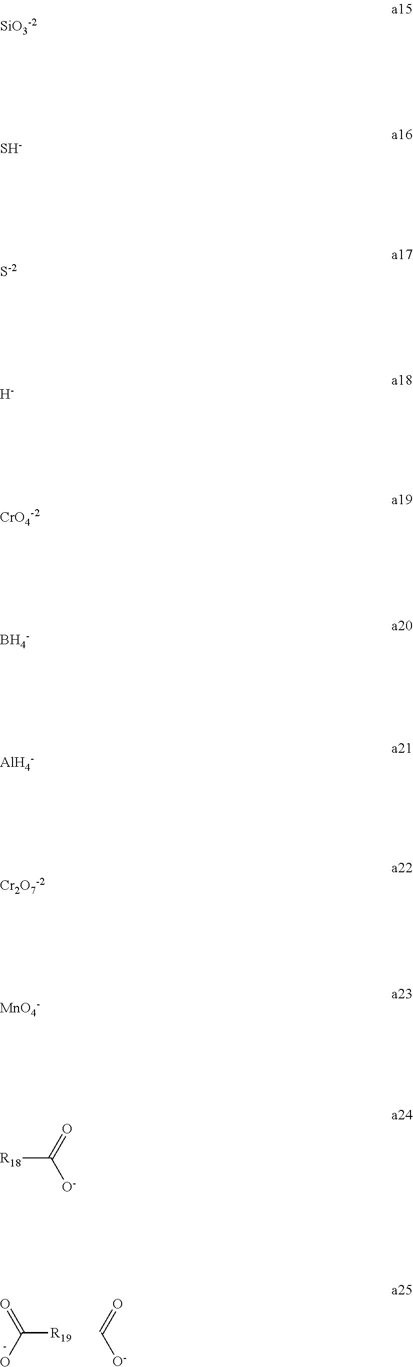

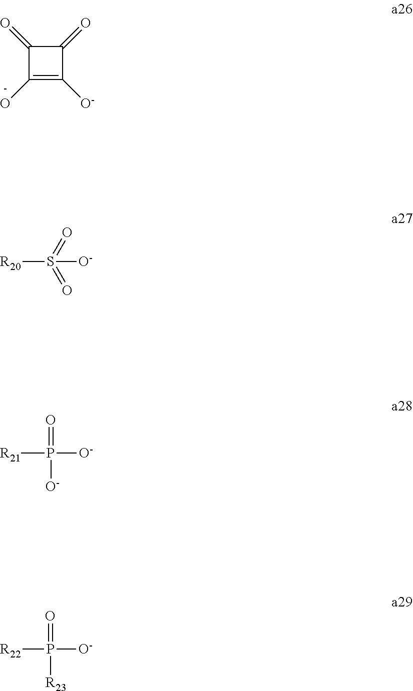

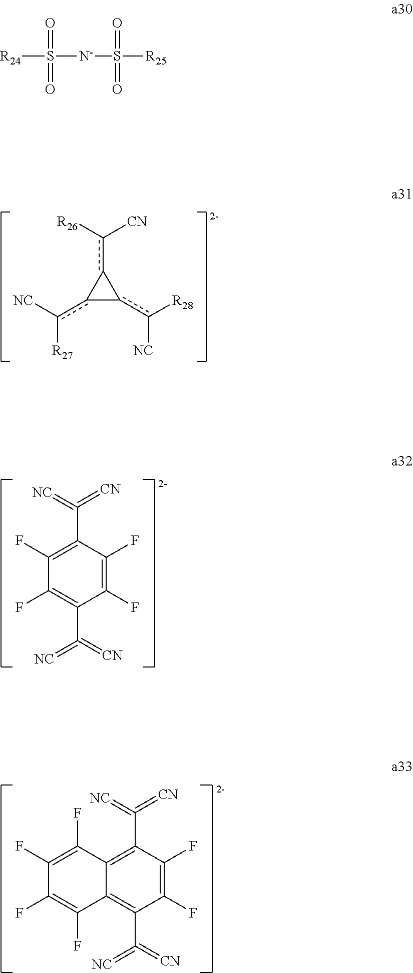

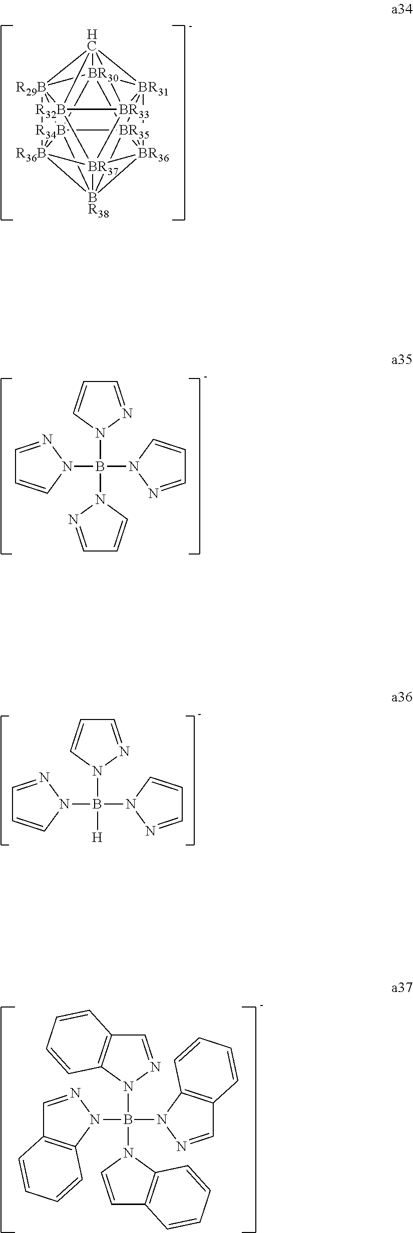

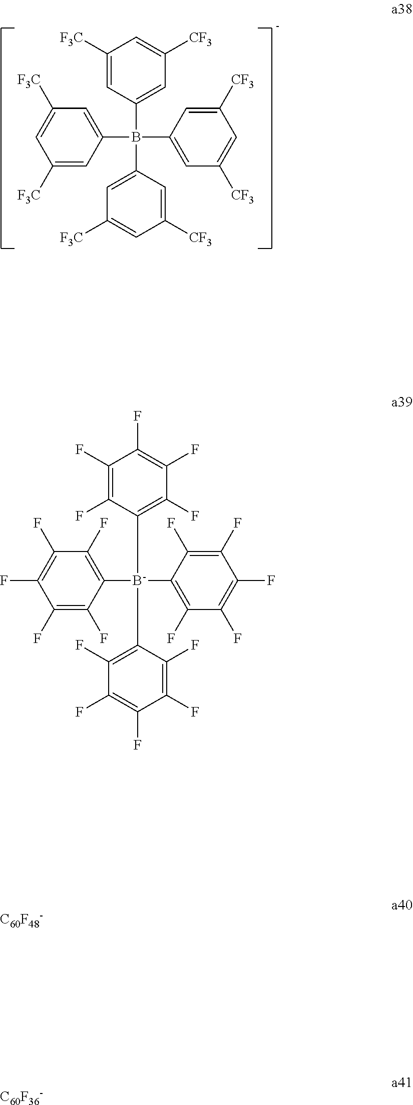

11. The coordination compound according to anyone of embodiments 1 to 10, wherein the coordination compound is selected from the group of c15 to c38, wherein c15 to c38 are:

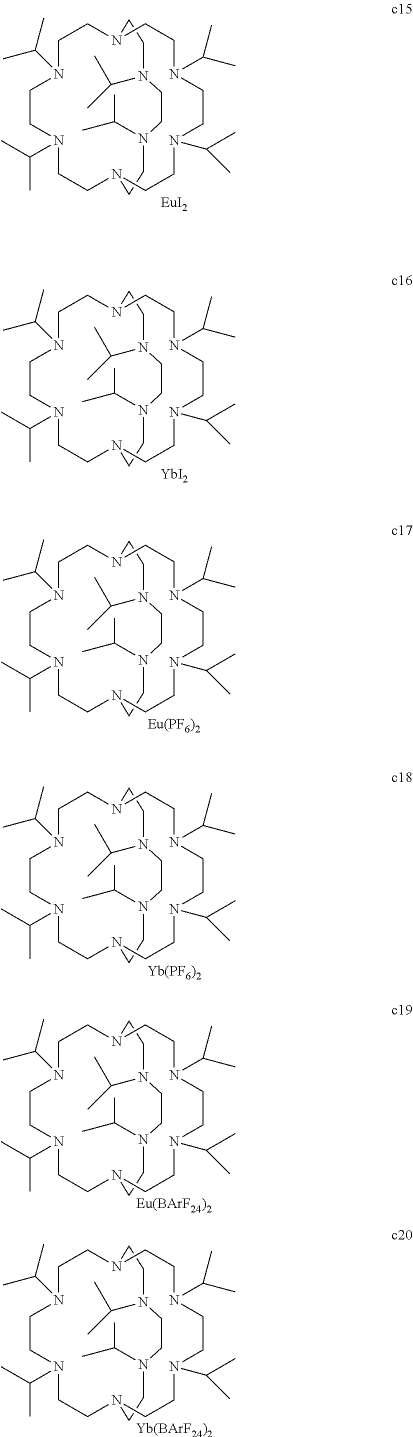

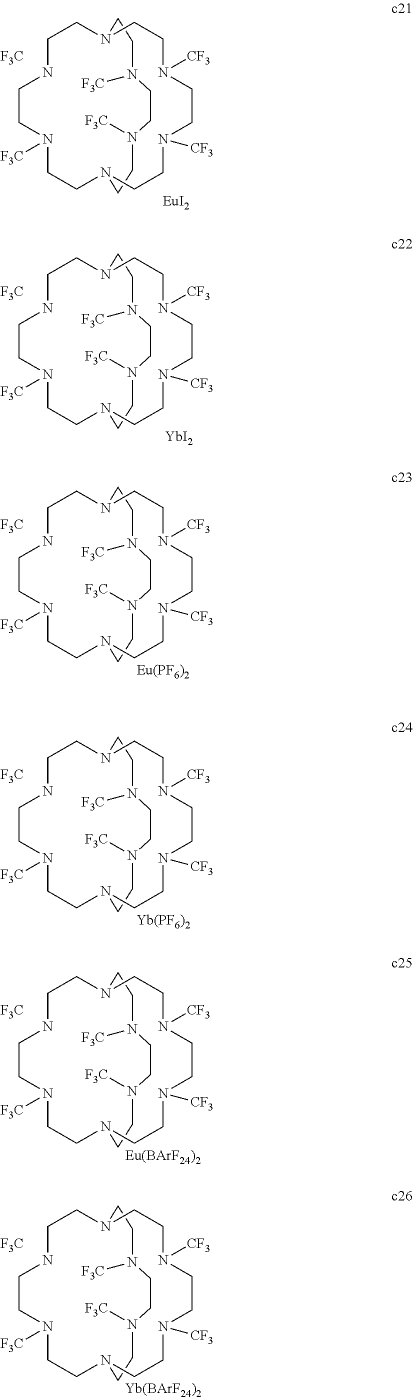

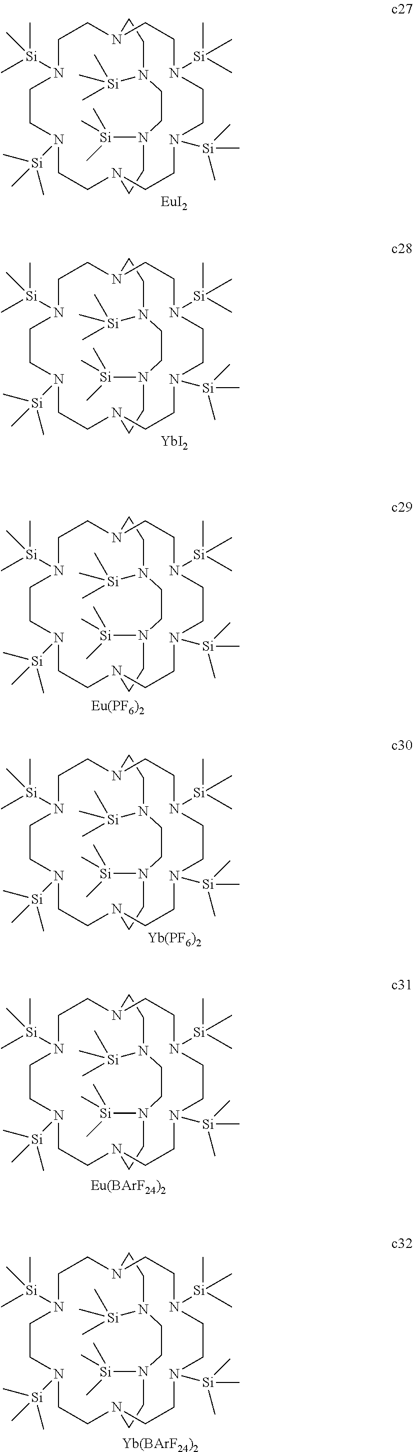

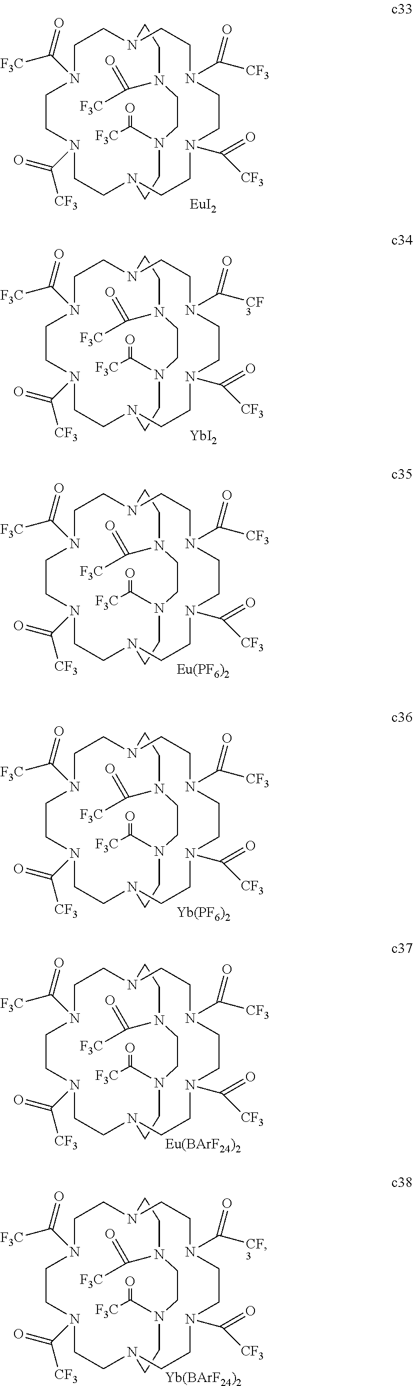

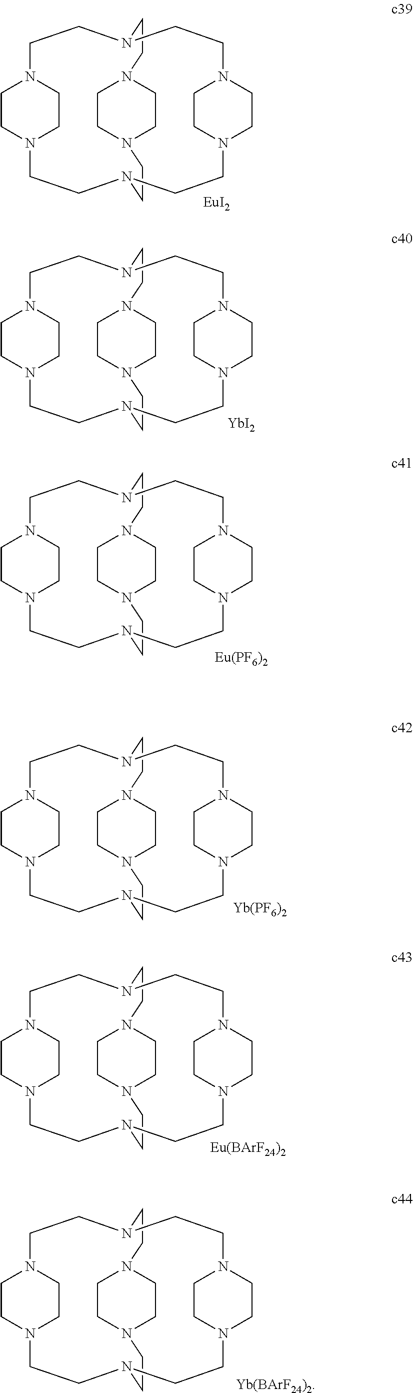

and
wherein BArF₂₄ represent the anion a38.
12. A mixture comprising
a second electrically neutral organic compound, and
the coordination compound according to any one of embodiments 1 to 11, wherein the coordination compound is imbedded into the at least one second electrically neutral organic compound,
wherein the second organic compound has a triplet energy higher than 2.5 eV and/or wherein the coordination compound has a higher hole affinity compared to the second organic compound.
13. A compound comprising
   the coordination compound according to any one of embodiments 1 to 11, and a polymer with a molecular weight Mn above 1000 g/mol, wherein the coordination compound is covalently attached to the polymer backbone.
14. A contrast enhancement medium for magnet resonance tomography (MRT), the contrast enhancement medium comprising the coordination compound according to any one of embodiments 1 to 11, the mixture of embodiment 12 or the compound of embodiment 13.
15. An organic electronic device, comprising:
   a first electrode;
   a second electrode; and
   an organic layer arranged such that it is electrically interposed between the first and second electrodes, wherein the organic layer comprises the coordination compound according to any one of embodiments 1 to 11, the mixture of embodiment 12 or the compound of embodiment 13.
16. The organic electronic device of embodiment 15,
   wherein the organic electronic device is an optoelectronic device, the optoelectronic device being at least one of an organic light emitting diode, an organic photodetector, or a photovoltaic cell.
17. A method of forming an organic device, the method comprising:
   forming a layer of the coordination compound according to any one of embodiments 1 to 11, of the mixture of embodiment 12 or the compound of embodiment 13,
   wherein the layer is deposited from a gas phase, in particular using an evaporation and/or sublimation process, and/or by a solution-based process.
18. The method according to embodiment 17,
   wherein forming the layer comprises forming a first layer comprising the organic ligand and forming a second layer directly in contact with the first layer, wherein the second layer comprises a divalent lanthanide salt.
19. A method to synthesize the coordination compound according to any one of embodiments 1 to 11,
   wherein a divalent lanthanide salt and an organic ligand according to formula (1) or formula (2) are reacted at pressure greater than or equal to about 1 bar.
20. The method according to embodiment 19,
   wherein the divalent lanthanide salt is coordinated with an organic precursor and subsequently at least one of the groups R₁, R₂, R₃, R₄, R₅, R₆ is attached to the organic precursor.

1-23. (canceled)

24. A metal-organic coordination compound, wherein the coordination compound comprises at least one divalent lanthanide coordinated by an electrically neutral cyclic organic ligand according to formula 1: 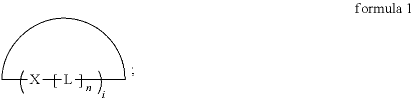
wherein
i is larger than 3; and
n is equal to 1, 2, or 3; and
L for each occurrence is independently selected from
divalent cyclic organic groups that can be substituted with substituents selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof and that are formed by removing two hydrogen atoms from an organic cyclic molecule that can be substituted with substituents selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof
arylenes, preferably 5- or 6-membered ring aromatic or heteroaromatic group, or
biradical fragments of 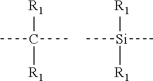
and
X is independently selected for each occurrence from the group of: 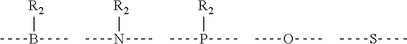
wherein R₁ and R₂ are hydrogen or any covalently bound substituents being identical or different in each occurrence and selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and
wherein R₁ and/or R₂ are at least in 3 occurrences not hydrogen, and
wherein two groups R₂ can be covalently linked with each other, thereby forming a further cyclic element,
wherein the cyclic organic ligand can be covalently linked with a polymer chain or a polymer backbone,
it also being possible that two cyclic organic ligands of formula 1 are covalently linked with each other by one or two divalent linking groups which divalent linking groups are formed of one R₁ of each of the two cyclic organic ligands of formula 1 that are covalently linked with each other,
wherein one R₁ and one R₂ are covalently linked with each other thereby forming a polycyclic ligand, and/or wherein at least two R₂ are covalently linked with each other, thereby forming a bridge to form a polycyclic ligand, wherein the coordination compound comprises at least one negatively charged anion, which is not covalently bound to the organic ligand, and has a molecular weight of at least 128 g/mol or includes more than 3 atoms and wherein the divalent lanthanide is Europium or Ytterbium wherein preferably the negatively charged anion has a molecular weight in the range of from 128 to 1000 g/mol.

25. A metal-organic coordination compound, wherein the coordination compound comprises at least one divalent lanthanide coordinated by a cyclic organic ligand according to formula 3: 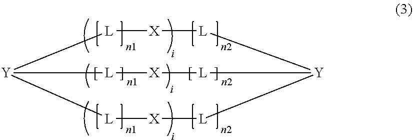
wherein
Y for each occurrence independently is B or N or P
X is independently selected for each occurrence from the group of: 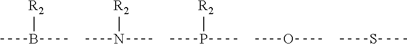
L for each occurrence independently is a divalent cyclic organic group that can be substituted with substituents selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof and that is formed by removing two hydrogen atoms from an organic cyclic molecule that can be substituted with sub stituents selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof, or is a divalent group —CR¹R¹— or —SiR¹R¹—
wherein R₁ and R₂ are hydrogen or any covalently bound substituents being identical or different in each occurrence and selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof
n1, n2, i independently are equal to 1, 2, 3, or 4,
wherein the coordination compound comprises at least one negatively charged anion, which is not covalently bound to the organic ligand, and has a molecular weight of at least 128 g/mol or includes more than 3 atoms the divalent lanthanide is Europium or Ytterbium, and the cyclic organic ligand can be covalently linked with a polymer chain or a polymer backbone,
wherein preferably the compound of formula 3 has one or more of the following features:
Y is B or N, wherein preferably R₂ is alkyl, alkoxy, carboxy, aryl, aryloxy or F,
all three structural elements 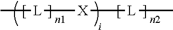
in the compound of formula 3 are identical,
divalent cyclic organic groups L for each occurrence independently are divalent cyclic organic groups that can be substituted and that are formed by removing two hydrogen atoms from neighboring carbon and/or nitrogen atoms in the ring of an organic cyclic molecule that can be substituted, n1 and n2 being 1,
cyclic organic groups are carbocyclic or heterocyclic groups, the heteroatoms being selected from P, N, Si, O, S
if in 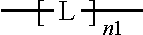
and/or 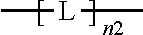
L is —CR¹R¹—, then n1 and/or n2 is 2, 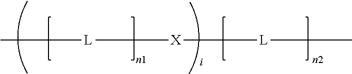
contains two groups X being 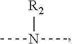
wherein both R₂ together form a group 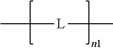
which is identical with group 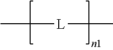
linking the two groups X,
i is 2,
the cyclic organic ligand according to formula 3 is electrically neutral.

26. The coordination compound according to claim 24,
wherein the coordination compound comprises at least one negatively charged anion, which is not covalently bound to the organic ligand, wherein the negatively charged anion has a molecular weight of at least 180 g/mol.

27. The coordination compound according to claim 24,
the cyclic organic ligand of formula 1 having a structure according to formula 2a, or 2c: 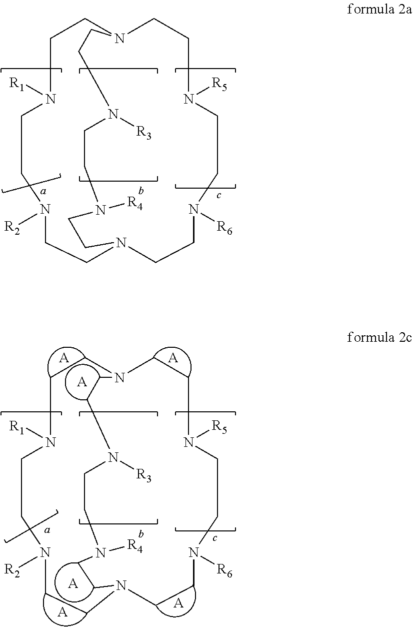
wherein
R₁, R₂, R₃, R₄, R₅, R₆ in formula 2a independently in each occurrence represent an organyl group, comprising at least one carbon atom, preferably at least one additional atom different from hydrogen, more preferably at least two carbon atoms, and R₁, R₂, R₃, R₄, R₅, R₆ in formula 2c independently in each occurrence represent hydrogen or an organyl group, comprising at least one carbon atom,
a, b, c are each independently an integer of 0 or more, 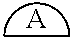
independently in each occurrence represents a divalent cyclic organic group, wherein in the coordination compound preferably a, b and c are each equal to 1 and/or wherein 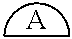
represent a 5- or 6-membered carboxylic or N-heterocyclic group.

28. The coordination compound according to claim 27, wherein R₁, R₂, R₃, R₄, R₅, R₆ are in each occurrence independently selected from the group of f1 to P8, wherein f1 to P8 are: 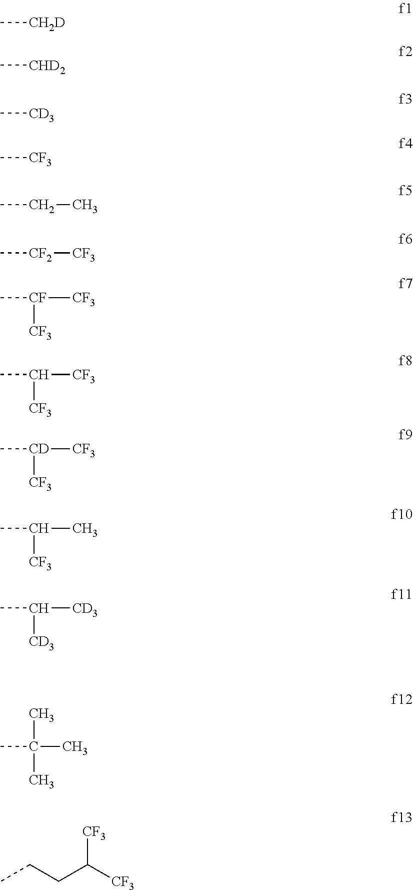 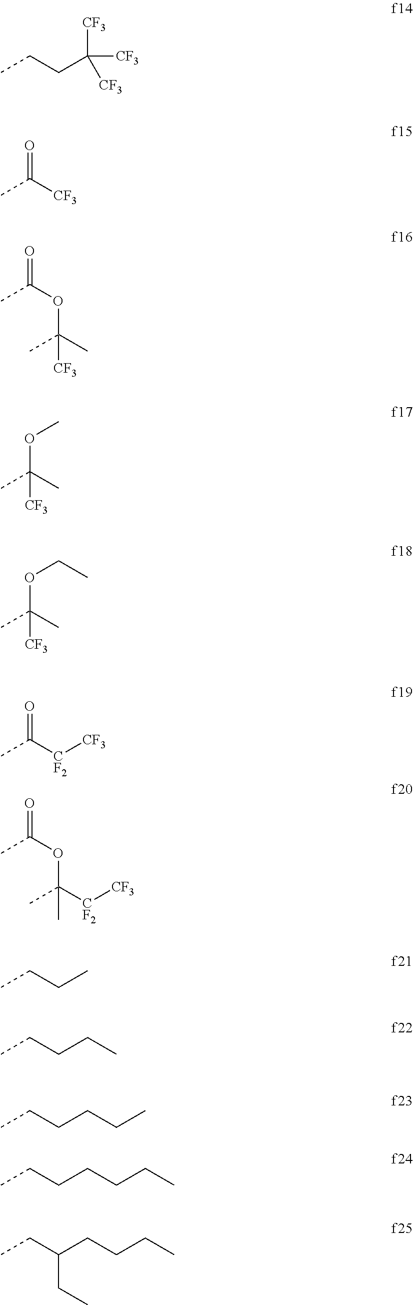 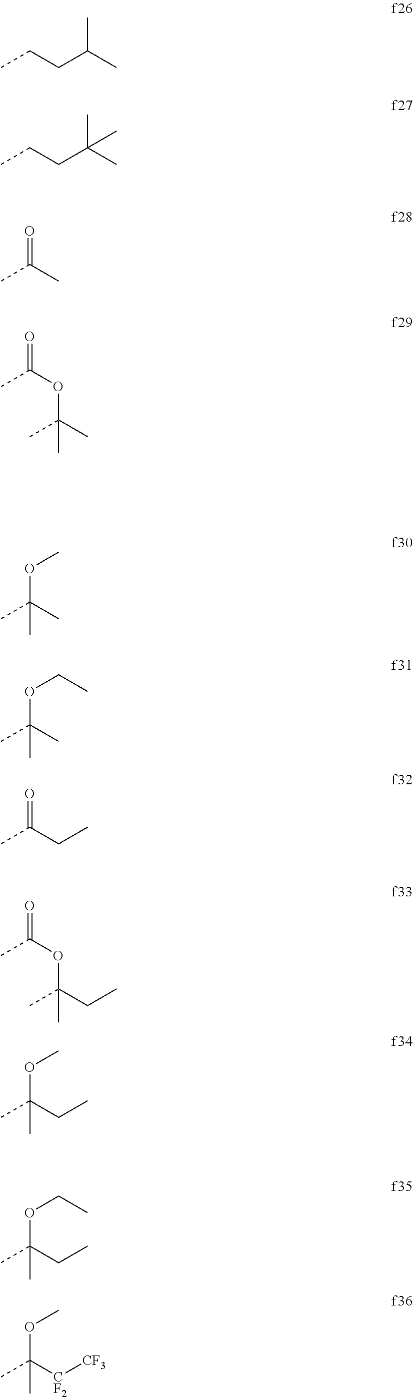 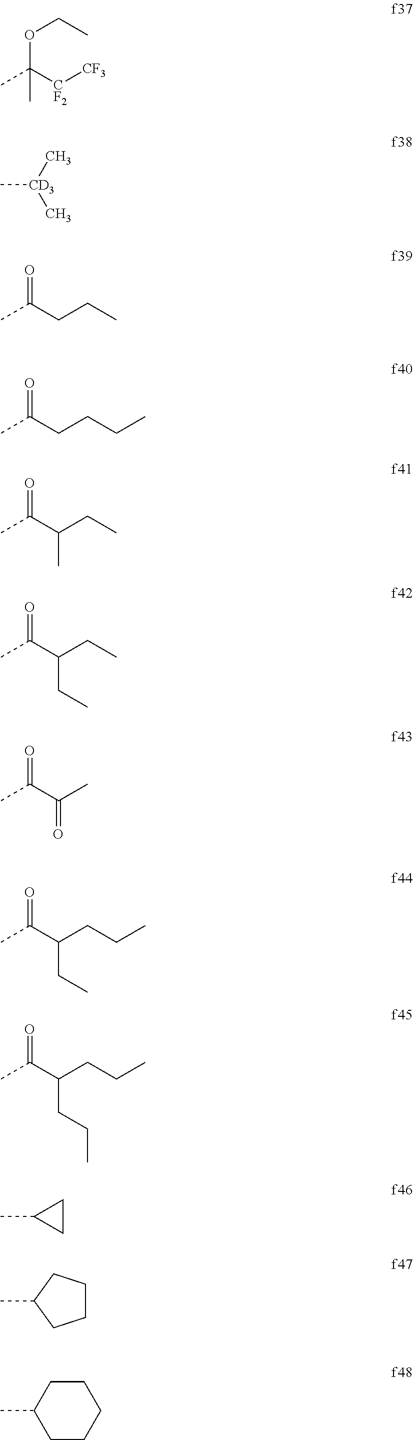 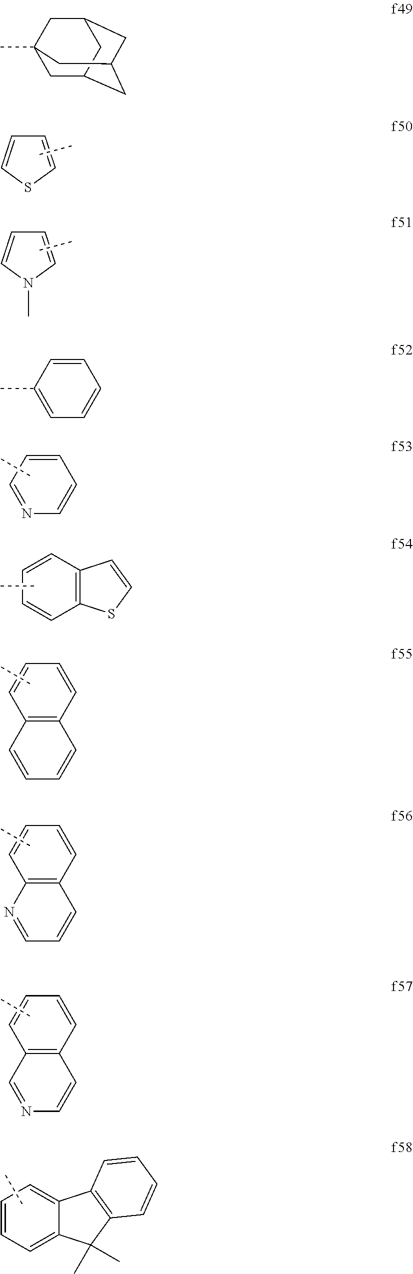 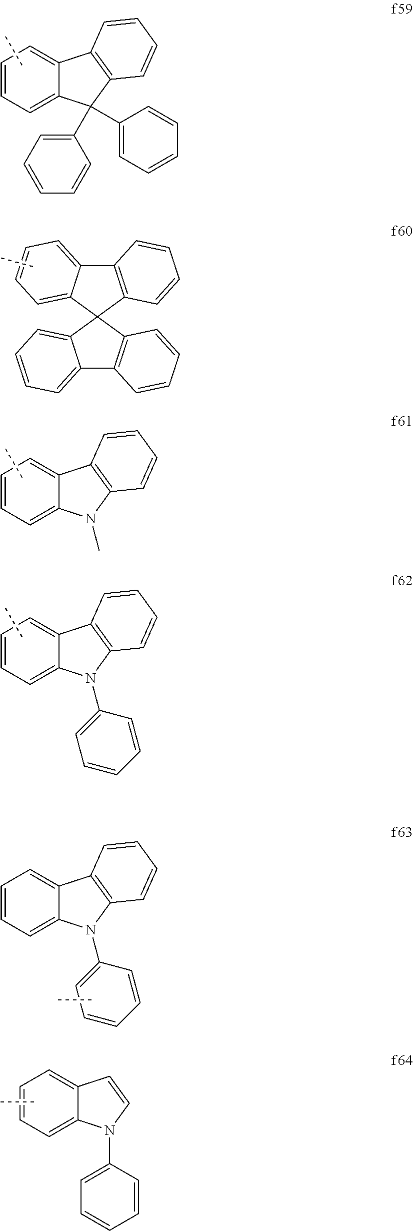 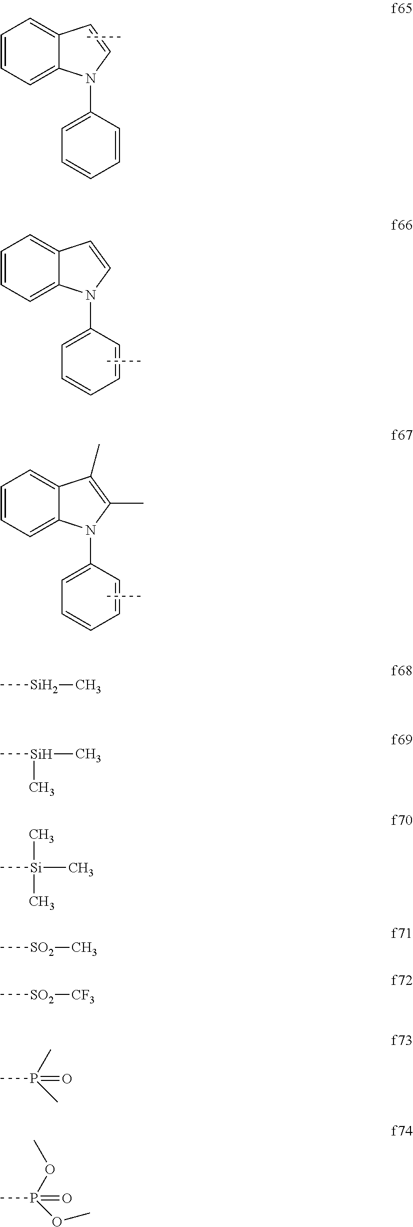 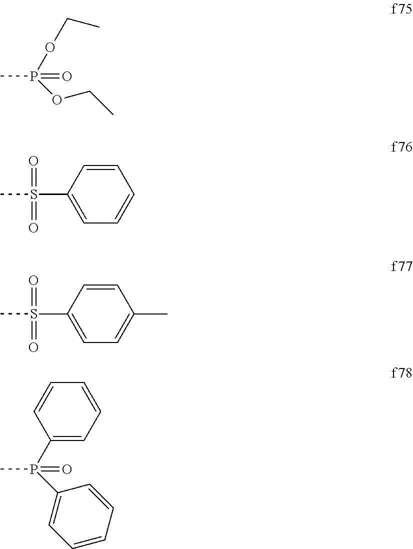
wherein a dash line represents the preferred connection point.

29. The coordination compound according to claim 24, wherein the negatively charged anion is at least one selected from the group of a5 to a42, wherein a5 to a42 are: 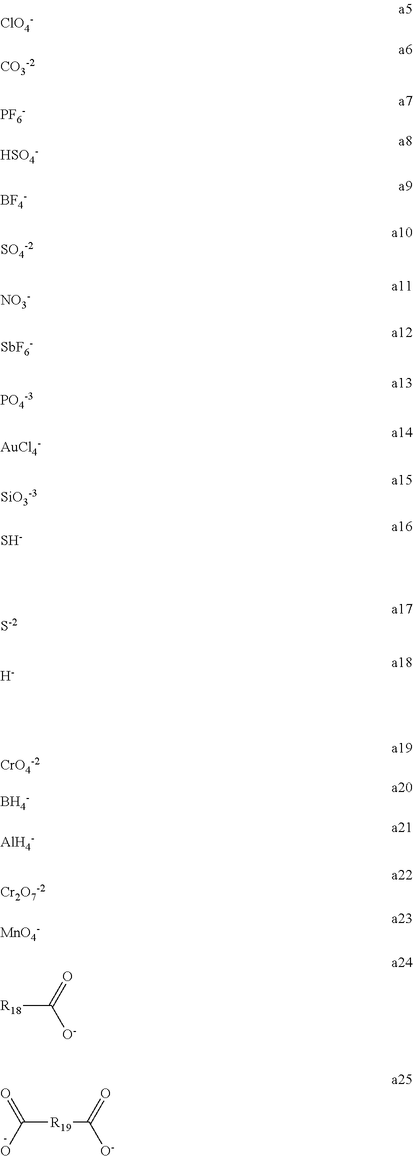 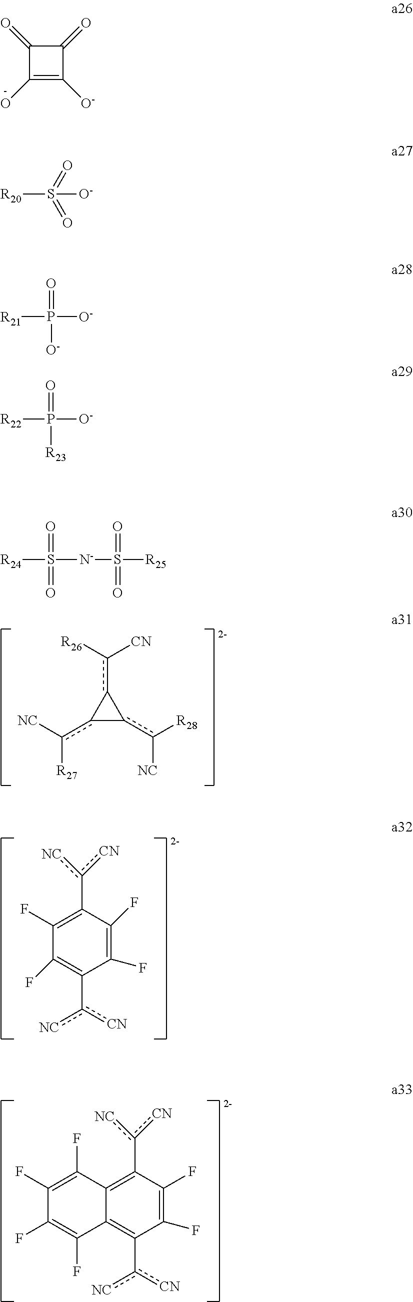 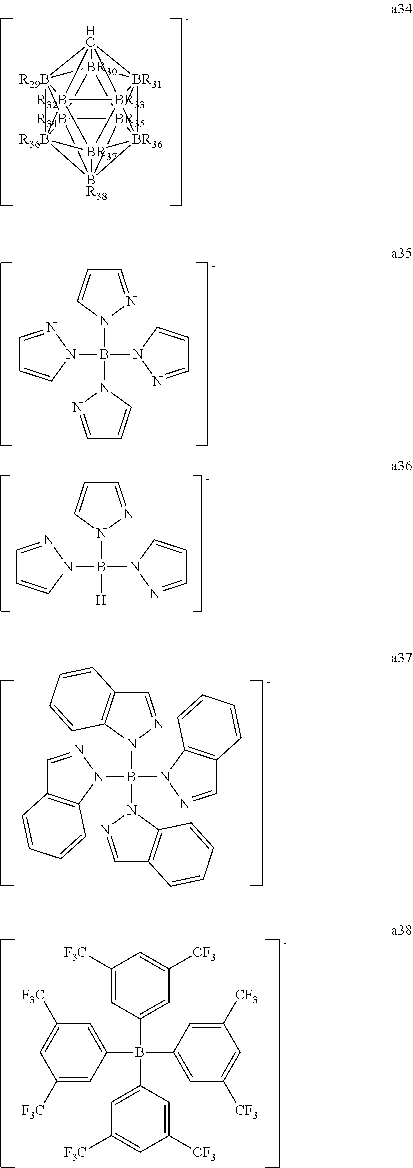 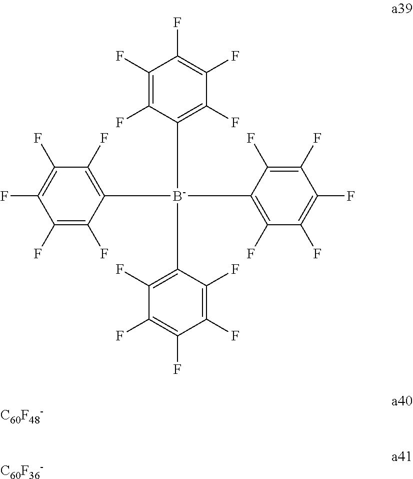
wherein R₁₈, R₂₀ to R₃₈ represent a monovalent group formed by removing of hydrogen atom from, preferably substituted, alkanes or, preferably substituted, arenes, or, preferably substituted, heteroarenes, Rig represents a divalent group formed by removing two hydrogen atoms from, preferably substituted, alkanes or, preferably substituted, arenes or, preferably substituted, heteroarenes and the free valences of which are not engaged in a double bond,
or wherein the coordination compound comprises at least one negatively charged anion, which is not covalently bound to the organic ligand, wherein the negatively charged anion is an organoboron compound containing at least three substituted or unsubstituted cyclic or heterocyclic organic groups covalently bound to the boron, or is at least one selected from the group of a1 to a18, wherein a1 to a18 are: 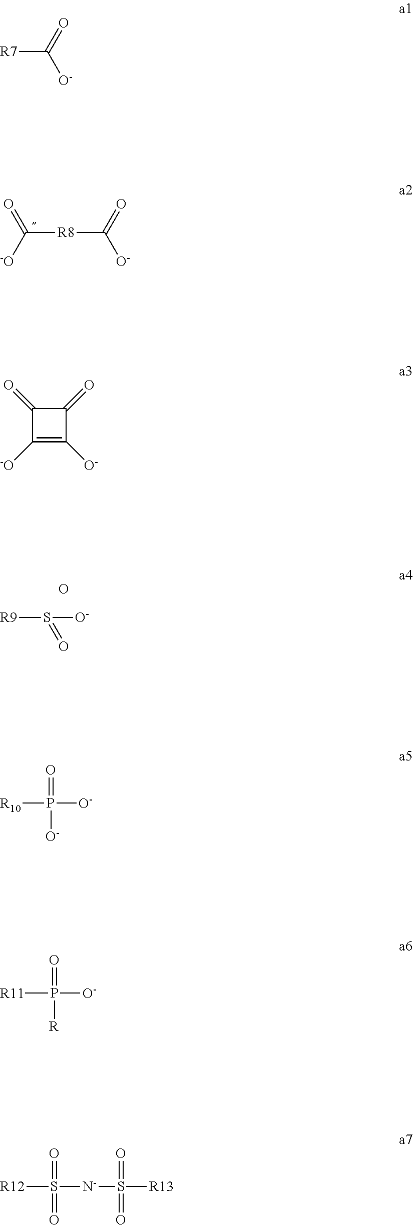 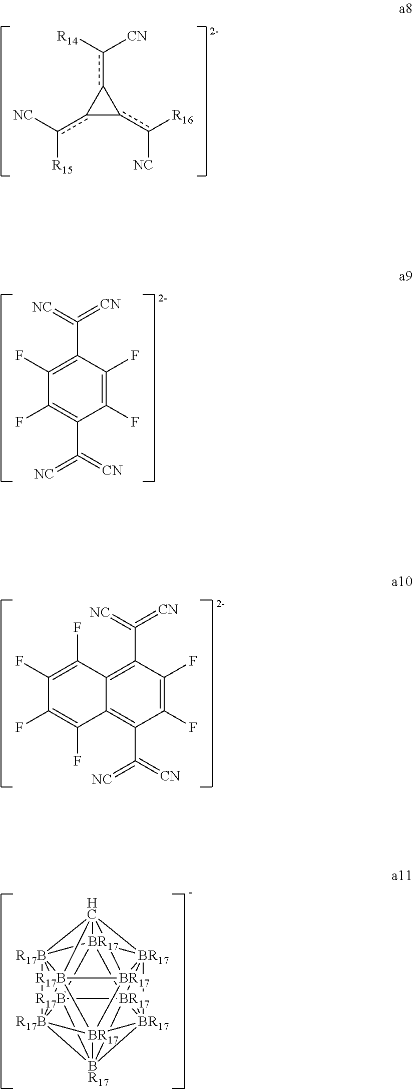 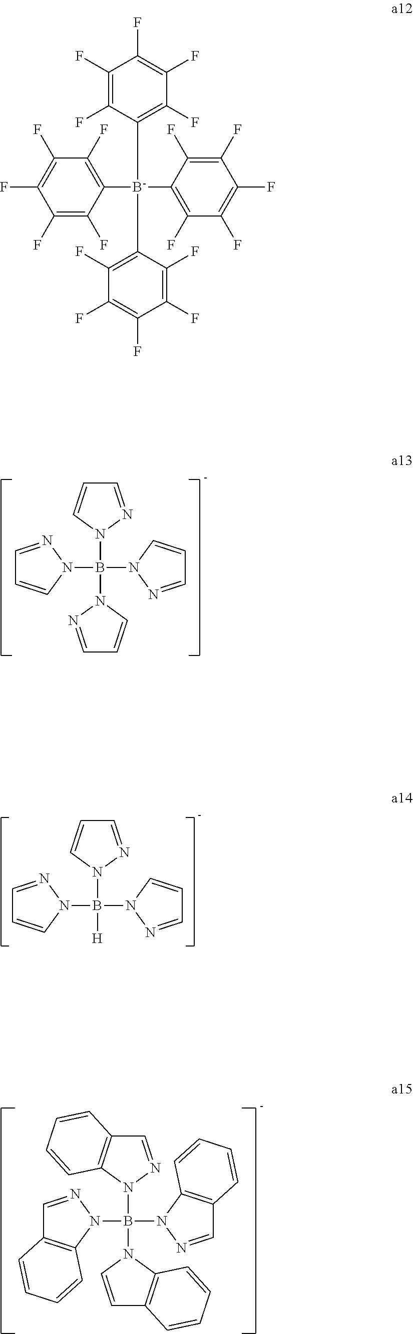 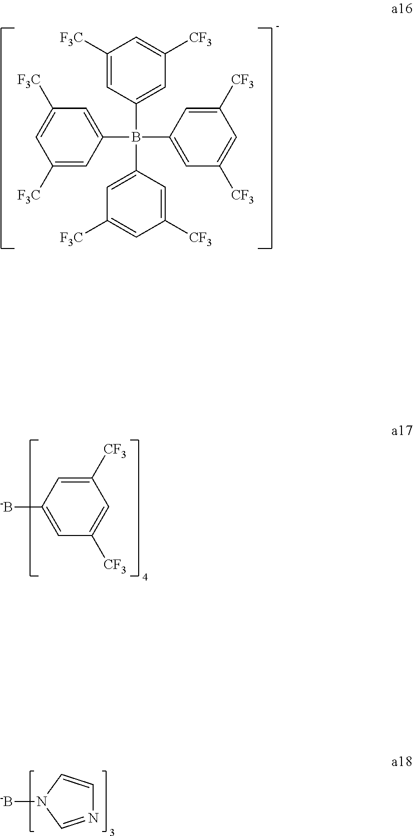
wherein R₇ is selected from hydrogen, deuterium, preferably substituted, preferably C₁-C₁₀ linear or branched alkyl, perfluorinated alkyl, partially fluorinated alkyl, or, preferably substituted, aryl, perfluorinated aryl, partially fluorinated aryl, or, preferably substituted, cycloalkyl, or, preferably substituted, alkenyl or, preferably substituted, alkynyl,
R₈ is selected from, preferably substituted, preferably C₁-C₁₀ linear or branched, alkanediyls, perfluorinated alkanediyls, partially fluorinated alkanediyls, or, preferably substituted, arylenes, perfluorinated arylenes, partially fluorinated arylenes, or, preferably substituted, cycloalkanediyls, or preferably substituted, alkenediyls or, preferably substituted, alkyndiyls,
R₉ to R₁₁ are selected independently from a group including hydrogen, deuterium, preferably substituted, preferably C₁-C₁₀ linear or branched, alkyl, perfluorinated alkyl, partially fluorinated alkyl, preferably substituted aryl, perfluorinated aryl, partially fluorinated aryl, preferably substituted, cycloalkyl, preferably substituted, alkenyl and, preferably substituted, alkynyl,
R₁₁-R₁₃ are selected independently from, preferably substituted, perfluorinated C₁-C₂₀ alkyl, preferably substituted, C₁-C₂₀ alkyl, preferably substituted, perfluorinated aryls or, preferably substituted, aryls,
R₁₄ to R₁₆ are selected independently in each occurrence from a group including F, CN, preferably substituted, perfluorinated aryl or preferably substituted, perfluorinated heteroaryl,
R₁₇ is selected independently in each occurrence from a group including hydrogen, deuterium, halogen, methyl group, trifluoromethyl-group,
or wherein the negatively charged anion is selected from
[MCl₄]— with M=Al, Ga, [MF₆]— with M=As, Sb, Ir, Pt, [Sb₂F₁₁]—, [Sb₃F₁₆]—, [Sb₄F₂₁]—, CF₃SO₃—, [B(CF₃)₄]—, [M(C₆F₅)₄]— with M=B, Ga, [B(ArcF₃)₄]—, [HO(B(C₆F₅)₃)₂]—, [CHB₁₁H₅Cl₆]—, [CHB₁₁F₅Br₆]—, [CHB₁₁Me₅Br₆]—, [CHB₁₁F₁₁]—, [CEtB₁₁F₁₁]—, [B₁₂Cl₁₁NMe₃]—, [Al(OR^{PF})₄]—, [F(Al(OR^{PF})₃)₂]—, triflate, perchlorate, tetrafluoroborate, tetraphenylborate, or hexafluoroanions,
or wherein the coordination compound is selected from the group of c15 to c44, wherein c15 to c44 are: 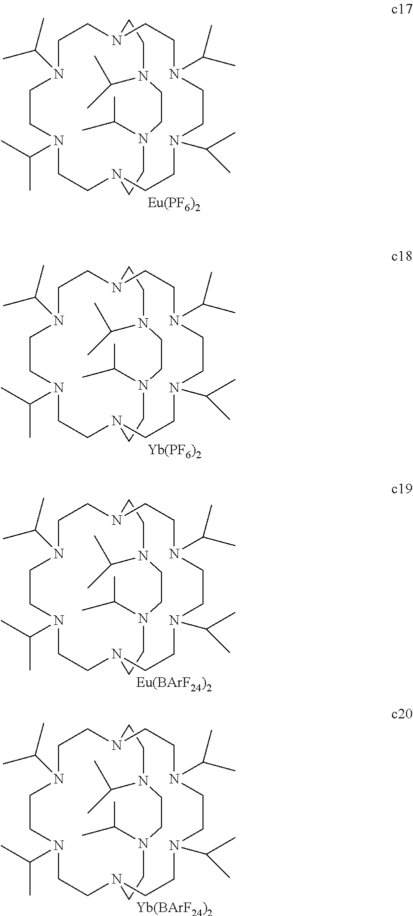 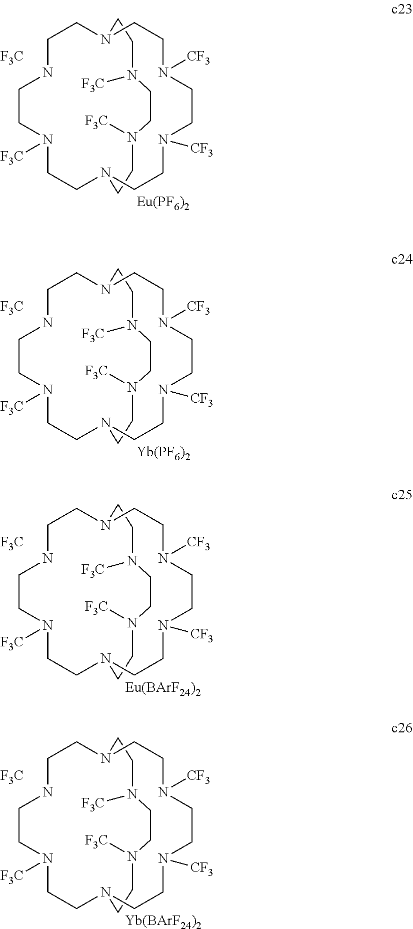 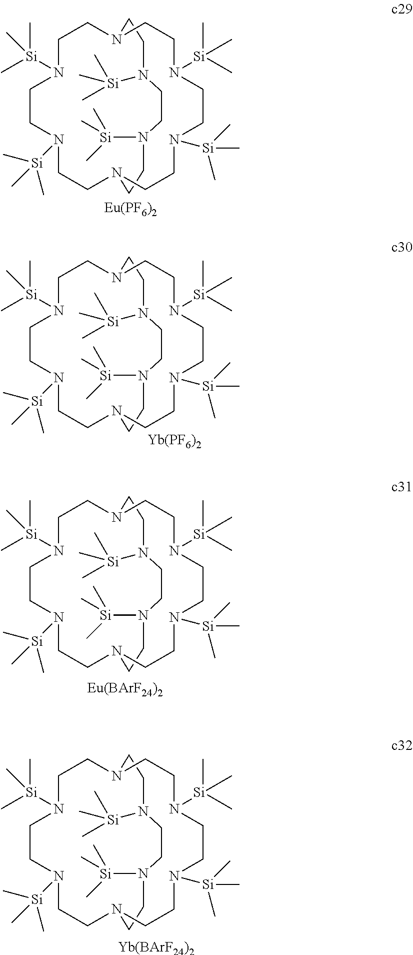 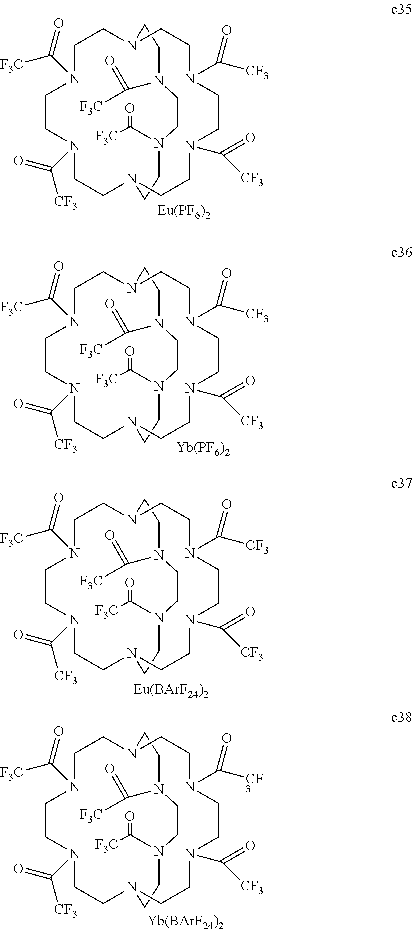 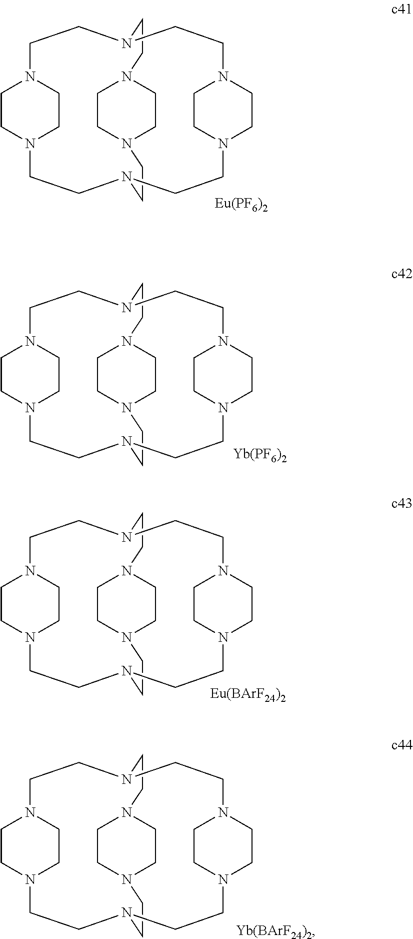
and
wherein BArF₂₄ represent the anion a38.

30. A metal-organic coordination compound, wherein the coordination compound comprises at least one divalent lanthanide coordinated by a cyclic organic ligand according to formula 3: 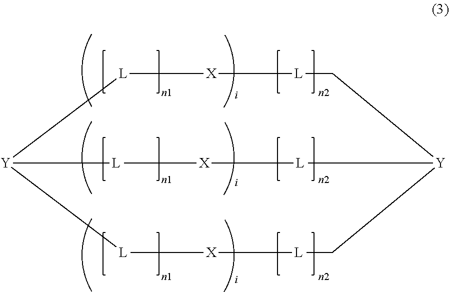
wherein
Y for each occurrence independently is B or B—R₂ or N or P
X is independently selected for each occurrence from the group of: 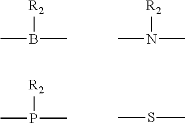
L for each occurrence independently is a divalent cyclic organic group that can be substituted with substituents selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof and that is formed by removing two hydrogen atoms from an organic cyclic molecule that can be substituted with substituents selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof or is a divalent group —CR¹R¹— or
—SiR¹R¹—
wherein R₁ and R₂ are hydrogen or any covalently bound substituents being identical or different in each occurrence and selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof
n1, n2, i independently are equal to 1, 2, 3, or 4,
the cyclic organic ligand can be covalently linked with a polymer chain or a polymer backbone and wherein the divalent lanthanide is Europium or Ytterbium,
with the exception of 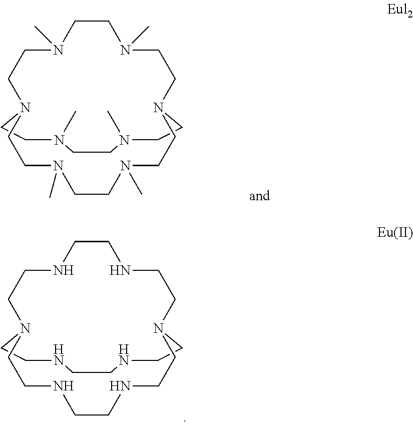 [SMILES: CN1CCN(C)CCN2CCN(C)CCN(C)CCN(CC1)CCN(C)CCN(C)CC2 ; C1CNCCN2CCNCCNCCN(CCN1)CCNCCNCC2]
wherein the cyclic organic ligand according to formula 3 can be electrically neutral.

31. The coordination compound according to claim 30,
the cyclic organic ligand was a structure according to formula 2a, 2b, 2c, 2d, 2e or 2f: 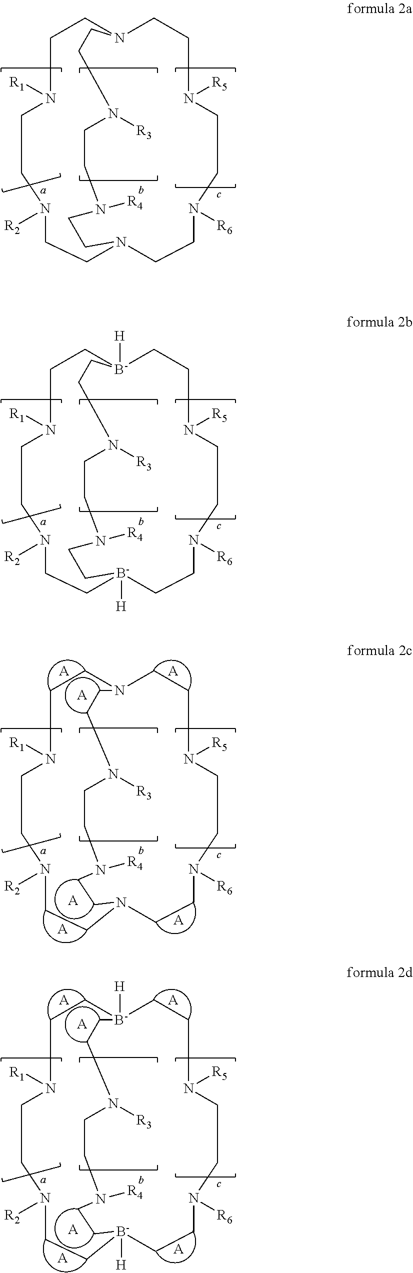 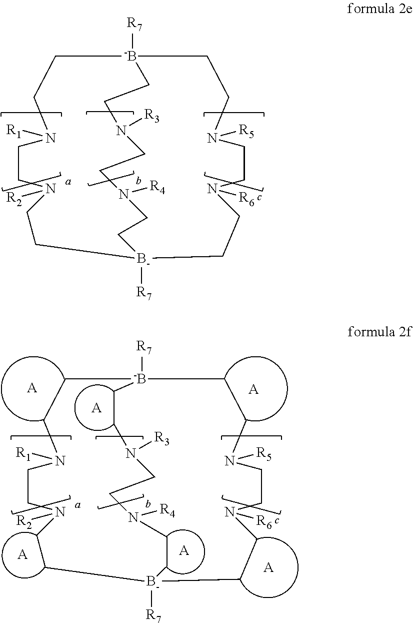
wherein
R₁, R₂, R₃, R₄, R₅, R₆ in formula 2a independently in each occurrence represent an organyl group, comprising at least one carbon atom, preferably at least one additional atom different from hydrogen, more preferably at least two carbon atoms, and R₁, R₂, R₃, R₄, R₅, R₆, R₇ in formula 2b, 2c, 2d, 2e, 2f independently in each occurrence represent hydrogen or an organyl group, comprising at least one carbon atom,
a, b, c are each independently an integer of 0 or more, 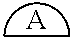
independently in each occurrence represents a divalent cyclic organic group, with the exception of

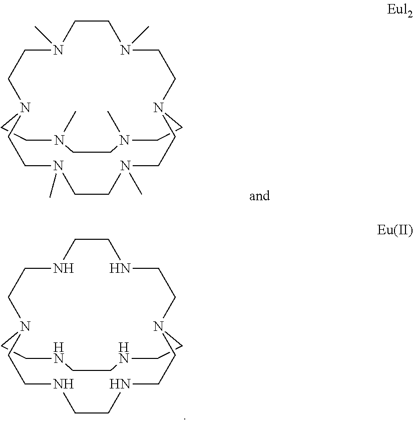

32. A mixture comprising
a second electrically neutral organic compound, and
the coordination compound according to claim 24,
wherein the coordination compound is imbedded into the at least one second electrically neutral organic compound,
wherein the second organic compound has a triplet energy higher than 2.5 eV and/or
wherein the coordination compound has a higher hole affinity compared to the second organic compound.

33. A compound comprising
the coordination compound according to claim 24, and
a polymer with a molecular weight Mn above 1000 g/mol, wherein the coordination compound is covalently attached to the polymer backbone.

34. A contrast enhancement medium for magnet resonance tomography (MRT), the contrast enhancement medium comprising the coordination compound according to claim 24, a mixture comprising
a second electrically neutral organic compound, and
the coordination compound,
wherein the coordination compound is imbedded into the at least one second electrically neutral organic compound,
wherein the second organic compound has a triplet energy higher than 2.5 eV and/or
wherein the coordination compound has a higher hole affinity compared to the second organic compound or compound comprising
the coordination compound, and
a polymer with a molecular weight Mn above 1000 g/mol, wherein the coordination compound is covalently attached to the polymer backbone.

35. An organic electronic device, comprising:
a first electrode;
a second electrode; and
an organic layer arranged such that it is electrically interposed between the first and second electrodes, wherein the organic layer comprises the coordination compound according to claim 24, a mixture comprising
a second electrically neutral organic compound, and
the coordination compound,
wherein the coordination compound is imbedded into the at least one second electrically neutral organic compound,
wherein the second organic compound has a triplet energy higher than 2.5 eV and/or wherein the coordination compound has a higher hole affinity compared to the second organic compound or a compound comprising the coordination compound, and
a polymer with a molecular weight Mn above 1000 g/mol, wherein the coordination compound is covalently attached to the polymer backbone or a metal-organic coordination compound, wherein the coordination compound comprises at least one divalent lanthanide coordinated by a cyclic organic ligand according to formula 3: 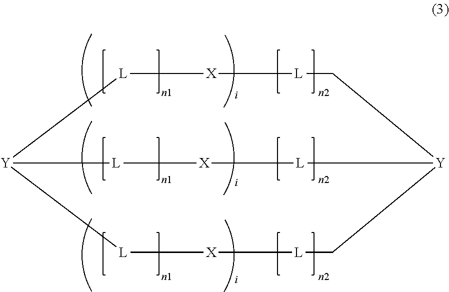
wherein
Y for each occurrence independently is B or B—R₂ or N or P
X is independently selected for each occurrence from the group of: 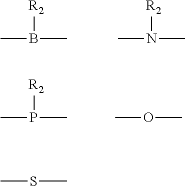
L for each occurrence independently is a divalent cyclic organic group that can be substituted and that is formed by removing two hydrogen atoms from an organic cyclic molecule that can be substituted, or is a divalent group —CR¹R¹— or
—SiR¹R¹—
wherein R₁ and R₂ are hydrogen or any covalently bound substituents being identical or different in each occurrence and
n1, n2, i independently are equal to 1, 2, 3, or 4
wherein the divalent lanthanide is Europium or Ytterbium, preferably wherein the cyclic organic ligand has a structure according to formula 2a, 2b, 2c, 2d, 2e or 2f: 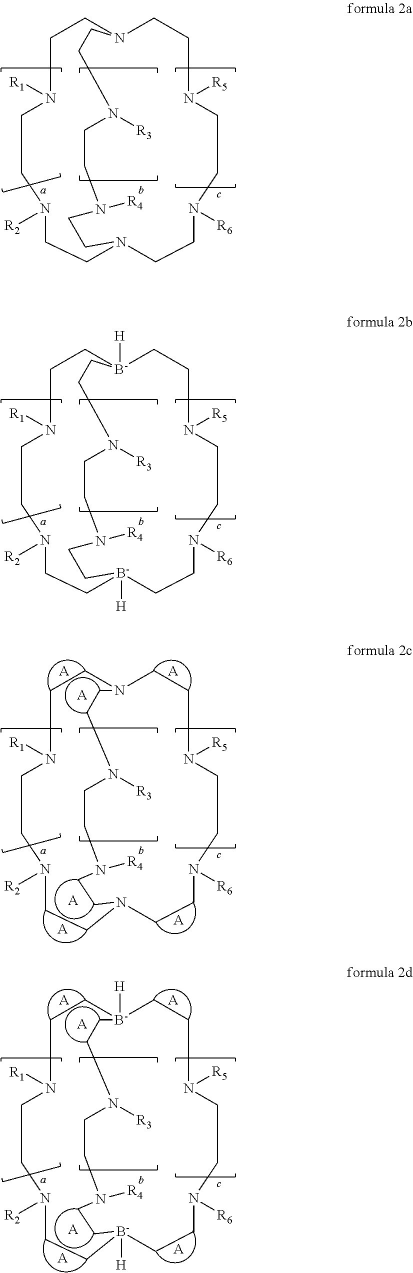 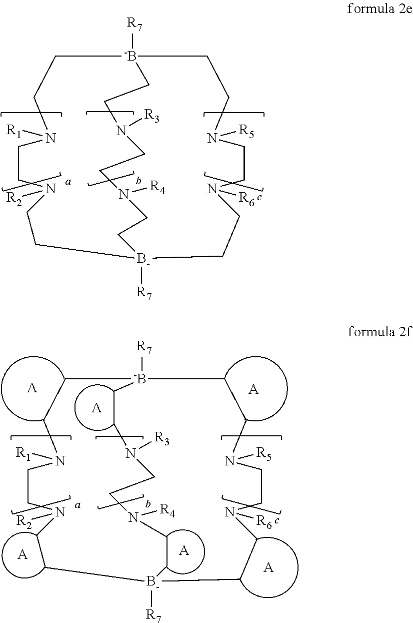
wherein
R₁, R₂, R₃, R₄, R₅, R₆ in formula 2a independently in each occurrence represent an organyl group, comprising at least one carbon atom, preferably at least one additional atom different from hydrogen, more preferably at least two carbon atoms, and R₁, R₂, R₃, R₄, R₅, R₆, R₇ in formula 2b, 2c, 2d, 2e, 2f independently in each occurrence represent hydrogen or an organyl group, comprising at least one carbon atom,
a, b, c are each independently an integer of 0 or more, 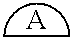
independently in each occurrence represents a divalent cyclic organic group with the exception of 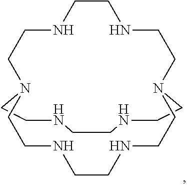 [SMILES: C1CNCCN2CCNCCNCCN(CCN1)CCNCCNCC2]
wherein preferably the organic electronic device is an optoelectronic device, the optoelectronic device being at least one of an organic light emitting diode, an organic photodetector, or a photovoltaic cell.

36. A method of forming an organic device, the method comprising:
forming a layer of the coordination compound according to claim 24, of a mixture comprising
a second electrically neutral organic compound, and
the coordination compound,
wherein the coordination compound is imbedded into the at least one second electrically neutral organic compound,
wherein the second organic compound has a triplet energy higher than 2.5 eV and/or
wherein the coordination compound has a higher hole affinity compared to the second organic compound or a compound comprising the coordination compound, and
a polymer with a molecular weight Mn above 1000 g/mol, wherein the coordination compound is covalently attached to the polymer backbone,
wherein the layer is deposited from a gas phase, in particular using an evaporation and/or sublimation process, and/or by a solution-based process,
wherein forming the layer preferably comprises forming a first layer comprising the organic ligand and forming a second layer directly in contact with the first layer, wherein the second layer preferably comprises a divalent lanthanide salt.

37. A method to synthesize the coordination compound according to claim 24, wherein a divalent lanthanide salt and an organic ligand according to formula (1) or formula (2) are reacted at pressure greater than or equal to about 1 bar,
and wherein preferably the divalent lanthanide salt is coordinated with an organic precursor and subsequently at least one of the groups R₁, R₂, R₃, R₄, R₉, R₆ is attached to the organic precursor.
